(19) United States
(12) Patent Application Publication
(10) Pub. No.: US 2009/0012148 A1
(43) Pub. Date: Jan. 8, 2009

(54) REDUCING CELLULAR CHOLESTEROL LEVELS AND/OR TREATING OR PREVENTING PHOSPHOLIPIDOSIS

(76) Inventors: Frederick R. Maxfield, Chappaqua, NY (US); Anthony Sauve, New York, NY (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/555,152

(22) Filed: Oct. 31, 2006

Related U.S. Application Data
(60) Provisional application No. 60/732,342, filed on Nov. 1, 2005.
(60) Provisional application No. 60/807,269, filed on Jul. 13, 2006.

(51) Int. Cl. A61K 31/4025; A61P 9/10; C12N 5/08; C12N 5/06; C07D 405/02; C07D 403/02

(52) U.S. Cl. 514/422; 548/517; 548/524; 435/375

(57) ABSTRACT

Compounds disclosed herein may be used in disclosed methods for reducing the amount of cholesterol in a cell, for treating a patient suffering from a disorder characterized by cellular accumulation of cholesterol (such as Niemann-Pick Disease Type C or atherosclerosis), and/or for treating or preventing phospholipidosis. In some embodiments, the compounds may include a pyrrolone or triazine moiety.

### CROSS-REFERENCE TO RELATED APPLICATION

This application claims the benefit of U.S. Provisional Application No. 60/732,342, filed Nov. 1, 2005, and of U.S. Provisional Application No. 60/807,269, filed Jul. 13, 2006, both of which applications are hereby incorporated herein by this reference.

### STATEMENT REGARDING FEDERALLY SPONSORED RESEARCH OR DEVELOPMENT

Support for research leading to subject matter disclosed in this application was provided in part by the National Institutes of Health Grant No. DK27083. Accordingly, the United States Government has certain rights with respect to subject matter of this application.

### BACKGROUND

Regulation of cellular cholesterol levels is essential for proper cell function and development. Cholesterol levels within a cell are regulated in part by cholesterol transport between various compartments and membranes. Proper distribution of cholesterol among cellular membranes is important for many biological functions, such as signal transduction and membrane trafficking. Cholesterol levels are also regulated by transport to extracellular receptors for removal of cholesterol from the cell. These cholesterol transport mechanisms have been widely studied, and defects in the regulation of cellular cholesterol levels have been linked to various diseases. Niemann-Pick disease is a class of inherited, lipid-storage diseases. Four types of Niemann-Pick disease are recognized: Types A, B, C and D. Types A and B are caused by a deficiency in sphingomyelinase activity leading to the build up of sphingomyelin in cells, often resulting in cell death. Patients suffering from type A Niemann-Pick disease often die by 2 to 4 years of age, whereas patients suffering from type B may survive into late childhood or adulthood. Type D Niemann-Pick disease (also known as the Nova Scotia variant) is allelic to type C and occurs in descendents of western Nova Scotians.

Niemann-Pick disease type C (NPC) is an autosomal recessive genetic disorder that causes an abnormal accumulation of cholesterol and other lipids in many cell types (1, 2). The most serious symptoms are caused by progressive neuronal degeneration, but the liver and other peripheral organs also exhibit defects. Although the time course can be variable, symptoms often develop in early childhood, and the disease is usually fatal by the teens. There have been attempts to develop treatments for NPC (3-8), but no effective therapy exists at present.

Two genes have been linked to the NPC defect in humans, although the precise mechanisms of action of these proteins are still under investigation. NPC1 is a multi-span membrane protein that is typically associated with late endosomes or lysosomes (9), degradative organelles that hydrolyze cholesterol esters brought into the cell via lipoproteins (10, 11). NPC1 has a sterol sensing transmembrane domain that is similar to that found in endoplasmic reticulum proteins that respond to changes in cellular cholesterol (12). The NPC1 protein facilitates transbilayer transport of some hydrophobic molecules, but it does not appear to transport cholesterol directly (13-16). NPC2 is a soluble lumenal protein that is found in late endosomes and is able to bind cholesterol (17-19). NPC2 may shuttle free cholesterol to the limiting membrane of the late endosomes and lysosomes, where NPC1 apparently plays a role in its export to other cellular sites (20). Loss of functional NPC1 or NPC2 causes accumulation of free cholesterol in endocytic organelles that have characteristics of late endosomes and/or lysosomes. These abnormal organelles will be referred to here as lysosome-like storage organelles (LSOs). The LSOs that are associated with NPC are quite similar to the LSOs associated with other hereditary glycosphingolipid storage disorders (often caused by the inability to metabolize a particular lipid) in that the storage organelles contain multi-layered internal whorls of membrane bilayers that contain cholesterol, sphingomyelin, and high amounts of bis-(monoacylglycero)-phosphate (BMP), also known as lyso-bisphosphatidic acid (LBPA) (21, 22). Thus, even though these diseases arise from different genetic defects, certain aspects of the cellular phenotype are very similar. Several lines of evidence point to a defect in cholesterol transport in NPC, although defects in transport of other lipids may also play an important role (23). NPC cells show abnormally high levels of unesterified cholesterol, which accumulates mainly in the LSOs. The accumulation of cholesterol can be detected using filipin, a fluorescent detergent that binds to free cholesterol in membranes (24). In wild type cells, excessive cholesterol delivered to cells from endosomes is either exported from the cell to extra-cellular acceptors or it is esterified by acyl co-A: cholesterol acyl transferase (ACAT), an enzyme localized in the endoplasmic reticulum (25). Despite the high content of free cholesterol in LSOs, the plasma membranes of NPC cells in culture actually have lower cholesterol content than normal cells (26) and a defect in cholesterol efflux to extra-cellular acceptors (27). Furthermore, there is a defect in delivery of lipoprotein-derived cholesterol for esterification by ACAT (28, 29). These characteristics suggest that cholesterol efflux from late endosomes is impaired in NPC cells.

Several different mutations are found in the NPC1 gene, which is responsible for about 95% of NPC disease in humans (13, 30-33). The correlation between the molecular defect and the age of onset of severe symptoms is not clear. The clinical presentation of NPC disease ranges from late-onset or mild symptoms in adults to early onset with acute symptoms in infants (34, 35). This indicates that other factors in the genetic background can partially ameliorate the disease. Similarly, studies of cultured cells have shown that over-expression of various proteins that affect membrane traffic can reduce cholesterol accumulation. In particular, over-expression of the small regulatory GTPases, Rab7 and Rab9 (36-38) reduces sterol accumulation in cultured fibroblasts. Since these proteins regulate many aspects of cellular membrane traffic, they may not be good therapeutic targets. Nevertheless, the differences in age of onset in humans and the effects of over-expression of exogenous genes both indicate that pharmacological treatments might be developed to ameliorate symptoms even if the precise functions of the NPC proteins are not restored.

Phospholipidosis is a condition in which there is an excess accumulation of phospholipids in bodily tissues. The excess accumulation of phospholipids is thought to be linked to alterations in the synthesis and/or metabolism of phospholipids. Phospholipidosis can occur when certain drugs are administered to a patient. For example, amiodarone, perhexyline, fluoxetine, and gentamicin can cause phospholipidosis when administered to human patients. See M. J. Reasor et al. Exp. Biol. Med. 2001, 226, 825. Since excess accumulation of phospholipids is an undesirable side-effect of certain drugs, compositions and methods of treating drug-induced phospholipidosis would be highly desirable.

Therefore, the need exists for a treatment for Niemann-Pick disease and other diseases caused by defective regulation of cellular cholesterol levels. The need also exists for a method of treating or preventing drug-induced phospholipidosis. The present invention fulfills these needs and has other related advantages.

### SUMMARY

One aspect of the present invention relates to compounds and pharmaceutical compositions that are useful for reducing the amount of cholesterol in a cell. In certain instances, the compounds of the invention comprise a pyrrolone or triazine moiety. Another aspect of the present invention relates to a method of treating a patient suffering from a disorder characterized by cellular accumulation of cholesterol. In certain instances, the invention relates to a method of treating Niemann-Pick Disease Type C or atherosclerosis. Another aspect of the present invention relates to a method of reducing the amount of cholesterol in a cell by exposing a cell to a compound of the invention. In certain instances, the method comprises exposing a cell to a compound comprising a pyrrolone or triazine moiety. Another aspect of the present invention relates to a method of treating or preventing drug-induced phospholipidosis.

### BRIEF DESCRIPTION OF THE FIGURES

FIG. 1 depicts the results of filipin binding assays in which wild type CHO cells (TRVb1) and NPC1 mutant CHO cells (CT60) were plated in 384 well plates and grown in regular growth medium for 48 h. Cells were washed with PBS, fixed with 1.5% PFA and stained with filipin. Images were acquired at 10× magnification for 2 positions per well using 360/40 nm excitation and 480/40 nm emission filters with a 365 DCLP filter. (A) Filipin stained image of TRVb1 cells; (B) filipin stained image of CT60 cells. Bar=30 μM. Images were analyzed using average filipin intensity and LSO Compartment Ratio. (C) histogram of average filipin intensity values; (D) histogram of LSO Compartment Ratio values.

FIG. 2 depicts the results of filipin binding assays in which cells were fixed with PFA and labeled with filipin. (A) Images acquired using Discovery-1 automated fluorescence microscope at 10× magnification using 360/40 nm excitation and 480/40 nm emission filters with a 365 DCLP filter. (B) Images after correction for shading and background. (C) High threshold setting used to identify LSO compartment. (D) Low threshold used to include entire cell area. Bar=20 μM.

FIG. 3 depicts the results of filipin binding assays in which CT60 cells were grown in growth medium overnight and treated with either solvent (A) or 10 □M hit compound (1-a-13) (B) in screening medium. After 20 h incubation, cells were washed with PBS, fixed with PFA, and stained with filipin. Images were acquired at 10× magnification. Bar=25 μM.

FIG. 4 depicts filipin-stained images of the CT60 cells affected by addition of some compounds that induced morphological changes and/or increased filipin intensity. (A) Compound 1-c-1 resulted in more dispersed fluorescence with no significant change in average filipin intensity. (B) Compound 1-c-2 induced more compact LSOs with no significant change in filipin intensity. (C) Compound 1-c-3 caused peri-nuclear clusters of LSOs in mutant cells to become more dispersed. (D) Compound 1-b-4 caused a significant increase in filipin intensity with filamentous or tubular staining. Bar=15 μM.

FIG. 5 depicts chemical structures of 14 compounds (1-a-1, 1-a-2, 1-a-3, 1-a-4, 1-a-5,1-a-6, 1-a-7, 1-a-8, 1-a-9, 1-a-10, 1-a-11, 1-a-12, 1-a-13 and 1-a-14) from the first library. Compounds 1-c-2 and 1-c-3 caused morphological changes, compound 1-b-2 caused increase in filipin intensity, and compound 1-b-4 increased filipin-intensity as well as induced morphological changes.

FIG. 6 depicts dose response graphs for 14 compounds (1-a-1, 1-a-2, 1-a-3, 1-a-4, 1-a-5, 1-a-6, 1-a-7, 1-a-8, 1-a-9, 1-a-10, 1-a-11, 1-a-12, 1-a-13 and 1-a-14) from the first library. CT60 and CT43 cells were seeded in 384 well plates in growth medium. After 24 h, compounds were added to achieve final concentrations of 123 nM, 370 nM, 1.11 μM, 3.33 μM and 10 μM in 4 wells per concentration, and cells were incubated overnight. Cells were then washed with PBS, fixed with PFA and stained with filipin. The LSO compartment ratio was determined for: (A) CT60 cells (average of 5 experiments) and (B) CT43 Cells (average of 3 experiments). The solid line indicates mean value for solvent control; the dashed line indicates −3 SD.

FIG. 7 depicts the results of a cytotoxicity analysis for 14 compounds (1-a-1, 1-a-2, 1-a-3, 1-a-4, 1-a-5, 1-a-6, 1-a-7, 1-a-8, 1-a-9, 1-a-10, 1-a-11, 1-a-12, 1-a-13 and 1-a-14). CT60 and CT43 cells were seeded in 384 well plates in growth medium. After 24 h compounds were added to achieve final concentrations of 5, 10 and 20 μM in 4 different wells per concentration, and cells were incubated for 24 h. An equivalent amount of DMSO was added in control wells. Cells were washed with PBS, fixed with PFA, and stained with nuclear stain Hoechst 33258. Images were obtained at 4× magnification using the Discovery-1 automated fluorescence microscope with 360/40 nm excitation and 480/40 nm emission filters and a 365 DCLP dichroic filter. Cells per well were counted, and the percentage reduction in cell number compared to the control is shown for: (A) CT60 cells (average of 4 experiments) and (B) CT43 Cells (average of 3 experiments) FIG. 8 depicts the chemical structures of 7 compounds (2-a-1, 2-a-3, 2-a-8, 2-a-9, 2-a-12, 2-a-13, 2-a-15) from the second library.

FIG. 9 depicts the effect of 7 compounds (2-a-1, 2-a-3, 2-a-8, 2-a-9, 2-a-12, 2-a-13, 2-a-15) from the second library. The dose dependence was determined as described in Figure. (A) CT60 cells (average of 5 experiments) and (B) CT43 cells (average of 3 experiments). The solid horizontal line indicates the mean value for solvent control; the dashed line indicates mean −3 SD.

FIG. 10 depicts the results of a cytotoxicity analysis for 7 compounds (2-a-1, 2-a-3, 2-a-8, 2-a-9, 2-a-12, 2-a-13 and 2-a-15). Cytotoxicity was measured by cell count and by LDH release for the 7 hit compounds from the secondary library. For cell count assay cells per well were counted as described in FIG. 7, and the percent reduction in cell number compared to the control was determined for (A) CT60 cells, and (B) CT43 cells. For the LDH cytotoxicity assay the percentage of cellular LDH released into the medium was measured in the presence of 7 hit compounds from the secondary library for (C) CT60 cells with reference to low (no compounds) and high (lysed cells) controls.

FIG. 11 depicts the effect of 7 compounds (2-a-1, 2-a-3, 2-a-8, 2-a-9, 2-a-12, 2-a-13, 2-a-15) from the second library at varying times. CT60 cells were seeded in 384 well plates in growth medium. After 24 h compounds were added to achieve final concentrations of 1.11 μM, 3.33 μM and 10 μM in 4 different wells/concentration and allowed to incubate for (A) 4 h, (B) 20 h, and (C) 48 h. Cells were washed with PBS, fixed with PFA, and stained with filipin. Images were obtained at 10× magnification using the Discovery-1 automated fluorescence microscope and 360/40 nm excitation and 480/40 nm emission filters equipped with a 365 DCLP filter. LSO compartment ratio was measured (average of 3 different experiments). The solid horizontal line indicates the mean value for solvent control; the dashed line indicates mean −3 SD.

FIG. 12 depicts the effect of 7 compounds (2-a-1, 2-a-3, 2-a-8, 2-a-9, 2-a-12, 2-a-13, 2-a-15) from the second library on U18666A-treated normal human fibroblasts. Normal human fibroblasts were plated in 384 well plates and grown in regular growth medium for 24 h, after which the cells were treated with compound U18666A (500 nM or 250 nM) in screening medium for 4 h. The cells were then further incubated overnight with hit compounds (10 μM) in the continued presence of U18666A. Finally, cells were washed three times with PBS, fixed with 1.5% PFA, washed with PBS and stained with filipin. Images were acquired using the Discovery1 microscope at 10× magnification and analyzed for the LSO ratio. Solid horizontal lines are the mean for U18666A-treated cells at each concentration, and the dotted horizontal lines indicate −3 SD.

FIG. 13 depicts the increase in cholesterol efflux from 25RA CHO cells, the parental cell line for the CT60 and CT43 cell lines and does not have an NPC mutation, incubated in 10 μM concentrations of various compounds.

### DETAILED DESCRIPTION

One aspect of the present invention provides compositions and methods for modulating cellular cholesterol levels. The compositions of the invention can be used to treat Niemann-Pick disease and other diseases involving defective regulation of cellular cholesterol levels. As described above, proper regulation of cellular cholesterol levels is essential for proper cell function and development. The effect a compound has on cellular cholesterol levels can be monitored using a filipin binding assay.

Described herein is an automated screening assay to identify compounds that partially reverse the phenotype of Niemann-Pick disease type C(NPC) mutant cells. The assay is based on binding of a fluorescent detergent, filipin, to free cholesterol. In untreated mutant cells, there is a large amount of free cholesterol as compared to control cell lines (42). The free cholesterol is highly concentrated in LSOs, organelles that are related to late endosomes, but also can contain protein markers that are usually not abundant in late endosomes (47). The molecular defect in NPC is a mutation or absence of one of two proteins associated with late endosomes, NPC1 and NPC2. These mutations cause a defect in efflux of cholesterol from late endosomes, resulting in high levels of accumulation of cholesterol in the LSOs.

Two screening assays were developed in order to evaluate the effect that a test compound has on modulating cellular cholesterol levels. The first assay employed a filipin-fluorescence intensity threshold sufficient to identify the areas in each image that contained cells. Using this procedure, the total integrated filipin fluorescence was obtained per cell area in each field. The intensity of staining of the plasma membrane was provided a clear distinction of cellular areas above background levels. However, a second assay was needed because the threshold value used to identify cellular areas did not clearly distinguish between LSO compartment and other cell areas. The assay parameter that was used was the total fluorescence divided by the number of pixels above threshold. This assay was designed to estimate the total cholesterol per cell, based on the approximation that cell area is constant under various conditions. Although it is believed that this assay provides a reliable measure of total cholesterol per cell, the results of the assay may be affected if cells spread or round up significantly in response to a treatment or if some pools of cholesterol differ in their ability to bind filipin.

Although this assay did not use sub-cellular information or single cell analyses, it permits use of the automated microscopy analysis. First, the microscopy system is a sensitive detector of relatively weak filipin fluorescence. Second, the measurement was restricted to the areas in each field that contained cells, which reduces the contribution from background. Finally, dividing total fluorescence power by the area covered by cells provides a correction for differences in cell density at the time of measurement.

We found that the filipin intensity per pixel provided enough discrimination of mutant versus wild type cells to be useful as a screening assay. This parameter was used as we adjusted experimental conditions, such as cell density and labeling conditions, to be used in the assay. Noting, however, that care should be exercised because in certain instances, the CT60 cells were separated from the control cell line by only a few standard deviations.

We obtained better discrimination of wild type versus mutant cells using the LSO compartment ratio assay, which used a threshold to identify areas in each field that contained heavily labeled organelles (i.e., the LSOs in the mutant cells). Since these are the sites of cholesterol accumulation in NPC cells, it would be expected that selective measurement of this pool of cholesterol would provide better discrimination of mutant versus wild type cells. This additional sensitivity is useful in identifying partially effective compounds in screening assays. The coefficient Z′(46) is a measure of the discriminatory power of a screening assay, and the LSO compartment ratio assay had a Z′ of 0.61 as compared to 0.22 for the average filipin intensity assay. A Z′ value greater than 0.5, is often considered to be adequate for the screening assays.

In the first screening assay, we identified 14 compounds that caused a significant decrease in the filipin labeling at 10 μM, including 3 compounds that produced significant reduction at 123 nM. The primary library was combinatorially synthesized from 126 templates. The observation that some of the compounds are effective at 123 nM indicates that it is likely that some of the compounds have high affinity interactions with their targets.

A second library of compounds was screened having Tanimoto similarity coefficients ranging from 0.3 to 0.96 (higher coefficient indicates higher similarity). The average Tanimoto coefficient of similarity was about 0.75. The screening assay employed lower doses of test compounds and placed a greater emphasis on nontoxicity than the assay performed on the first library. Even though the dosage of the test compound was reduced from 10 μM to 1 μM in the assay of the second library, the second library contained a higher fraction of selected compounds (0.18%) compared to the first library (0.1%). Thus, the selection of chemicals in the secondary library led to a significant enrichment in potential hits. Furthermore, several of the selected compounds had greater efficacy and lower toxicity than the compounds from the initial screen. The 7 compounds identified from the second library were based on 4 synthetic templates. Compounds 2-a-1, 2-a-9, 2-a-12 and 2-a-13 are based on triazines, and this class of compounds has been of significant interest in the field of medicinal chemistry. See (48-52).

The 7 compounds from the second library selected for further characterization can generally be divided into two groups. Compounds 2-a-1, 2-a-9, 2-a-12 and 2-a-13 (Group I) are based on a 1,3,5-triazine core, and this class of compounds has been of significant interest in the field of medicinal chemistry (52-56). The second group of compounds (Group II) have five membered ring heterocycle cores (2-a-3: a 2-thioxo-1,3-thiazolidin-4-one derivative, 2-a-15 contains a methine-linked pyrolle and pyrrol-2-one and 2-a-8 contains a 1,3-thiazole N-linked to a dihydropyrazole). Both groups of compounds are extensively substituted from the cores with Group I triazines bearing mostly aryl or cyclic amines (or bearing a hydrazino group). Group II are also aryl-substituted, with compound 2-a-3 featuring an interesting partially saturated diethyl-amino naphthalene moiety connected to the 2-thioxo-1,3-thiazolidin-4-one via a double bond. Compound 2-a-15 has three aromatic rings in extended conjugation while compound 2-a-8 incorporates 6 different ring systems, of which five are aromatic. Both Group I and Group II compounds appear to be highly conformationally restricted molecules with extensive unsaturation. Their peripheries tend to be very hydrophobic while their centers are more hydrophilic. We note this characteristic implies that they are a type of spatial amphiphile (hydrophobic outside-hydrophilic inside). Although there are hydrophilic groups at the peripheries in some cases (notably 2-a-13 nitro group), the consistent distribution of several hydrogen-bonding moieties to the cores of these structures suggests that the cores may assist specific recognition of their in vivo targets.

Although the assay was developed for use in CHO cell lines, it should be applicable, with minor modifications, for analyzing cholesterol accumulation in other cell types. An assay for cholesterol accumulation can potentially be useful not only for NPC but also for other glycolipid storage disorders. Although the underlying biochemical basis for the disorders vary, many of these disorders result in a similar phenotype that includes formation of internal membrane whorls in LSOs that contain sphingomyelin, lyso-bis-phosphatidic acid, and cholesterol (53, 54). This assay can be used, not only for chemical screens, such as the one described here, but also for molecular genetics screens such as RNAi knockdown and gene expressions. Using conventional methods, a few genes have been identified that can correct the NPC phenotype when over-expressed in cells (36, 38, 55). The screen we have described herein could be used for large scale gene expression screens.

The screening assays also identified compounds that increased filipin staining even above the levels found in the NPC mutant cells. Upon further investigation, some compounds that initially appeared to increase filipin staining were found to be fluorescent at wavelengths that overlapped the spectrum of filipin, and as such, their fluorescence was probably the basis for the increased fluorescence seen in the assay. However, several non-fluorescent compounds were also found to increase filipin staining in the NPC cells. We also found some compounds that created a significant change in the morphology of the compartments that are enriched in free cholesterol. In particular, compound 1-c-3 produced a large network of apparently tubular organelles that were labeled with filipin.

We also measured the cholesterol content of treated cells by a direct chemical method. Most of the selected compounds in both screens did reduce cellular cholesterol, although 3 of the initially selected compounds did not show a reduction in cholesterol in a gas chromatography analysis. Thus, in certain instances, it may be prudent to verify the results of filipin binding assays by independent chemical analyses.

It is thought, although not to be bound by a particular theory, that cholesterol efflux from late endosomes requires several steps. The efflux, like many steps of intracellular transport, is apparently mainly non-vesicular (56). NPC2 presumably plays a role in delivering cholesterol from the sites of hydrolysis of sterol esters to the limiting membrane of the organelles (57). NPC1, and presumably other proteins, would facilitate delivery of cholesterol from the limiting membrane to cytosolic carriers. These carriers, which have not been identified molecularly, would transport cholesterol to the plasma membrane or other organelles (58, 59). Total free cholesterol in the organelles could be reduced by increasing efflux to extra-cellular acceptors in the plasma and/or esterification of cholesterol by ACAT in the endoplasmic reticulum. Reduced uptake of cholesterol or reduced synthesis could also cause a reduction in cellular cholesterol during the incubation with compounds.

We assayed for reduction of the lipid BMP, which accumulates in NPC cells, using an analysis methods similar to average intensity and the LSO assay. None of the hit compounds from the secondary library produced a significant reduction in BMP labeling after 16 hour incubations (data not shown). Several of the hit compounds from the secondary library did cause a reduction in cholesterol accumulation in normal human fibroblasts treated with U18666A, which causes cholesterol accumulation in LSOs. This indicates that the compounds do not rely on the SCAP mutation or other special properties of the CHO cell lines used for the screen.

Although not to be bound by a particular theory, the effects of the test compounds could be directly on the LSOs, but there may be indirect effects as well. For example, over-expression of Rab4, a small GTPase that is normally associated with sorting endosomes or the endocytic recycling compartment, can partially correct the NPC phenotype (60).

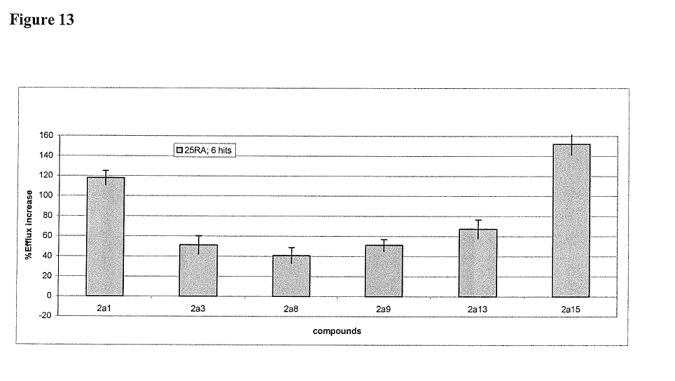

The compounds identified in the screening assays are effective in reducing cholesterol accumulation at concentrations at which they are non-toxic to cultured NPC1 cells. Further, several of the compounds (FIG. 13) should be effective in lowering the amount cholesterol in normal cells since they demonstrate efficacy in promoting cholesterol efflux in 25RA CHO cells. The compounds of the invention could also be used for studying cellular mechanisms that regulate cholesterol levels. For example, the compounds of the invention may be modified with photo-reactive groups for labeling binding partners or linkage to biotin for affinity purification. Further, although not to be bound by a particular theory, the compounds of the invention may be effective in reducing cholesterol uptake by the cell and/or inhibiting cholesterol biosynthesis. Another aspect of the invention relates to methods of treating or preventing drug-induced phospholipidosis. Drug-induced phospholipidosis can occur as a side effect when a pharmaceutical agent is administered to a patient. For example, the following pharmaceutical agents can cause phospholipidosis: ABT-770, AC-3579, amantadine, ambroxol, amikacin, amiodarone, amitryptilline, AY-9944, azithromycin, benzamide, boxidine, bromhexine, chlorocyclizine, chloroquine, chlorphentermine, chlorpromazine, citalopram, cloforex, clomipramine, clozapine, compound 200-15, cyclizine, DMP 777, erythromycin, fenfluramine, fluoxetine, fluvoxamine, gentamicin, hydroxyzine, IA-3, imipramine, iprindole, LY281389, maprotiline, meclizine, mepacrine, NE-10064, netilmicin, norchlorcyclizine, noxiptilin, perhexyline, phentermine, PNU-177864, promaxine, promethazine, propanolol, RMI 10.393, sertraline, tamoxifen, thioridazine, tilarone, tobramycin, trimipramine, triparanol, triperennamine, trospectomycin, zimelidine, 1-chloroamitryptiline, and 4,4′-diethylaminoethoxyhexestrol. See M. J. Reasor et al. Exp. Biol. Med. 2001, 226, 825; M. J. Reasor et al. Expert Opin. Drug Saf. 2006, 5, 567; Lüillmann-Rauch R., Drug-induced Lysosomal Storage Disorder, in LYSOSOMES IN BIOLOGY AND PATHOLOGY, Vol. 6., pp. 49-130 (Dingle et al. eds., Amsterdam: North-Holland, 1979); Kodavanti et al. Pharmacol. Rev. 1990, 42, 327; M. J. Reasor. Cationic Amphiphilic Drugs, in COMPREHENSIVE TOXICOLOGY, Vol. 8, TOXICOLOGY OF THE RESPIRATORY SYSTEM pp. 555-566 (Sipes et al. eds., New York: Elsevier Science, 1997); and Sawada et al. in Toxicol. Sci., 2005, 83, 282 and Toxicol. Sci. 2006, 89, 554. Various drugs with a cationic lipophilic structure can also cause drug-induced phospholipidosis. Such drugs often have a hydrophilic region comprising at least one primary or substituted nitrogen group that is positively charged at physiological pH, and a hydrophobic region comprising an aromatic and/or cycloaliphatic group optionally substituted with a halogen. See M. J. Reasor et al. Exp. Biol. Med. 2001, 226, 825.

Procedures for identifying compounds that cause phospholipidosis are known in the art. See, e.g., H. Sawada et al. Toxicol. Sci. 2005, 83, 282. Since excess accumulation of phospholipids is an undesirable side-effect of certain drugs, one aspect of the present invention relates to a method of treating or preventing drug-induced phospholipidosis by administering to a patient in need thereof a therapeutically effective amount of a compound of any one of formulae I-IX described herein. In certain instances, the patient's drug-induced phospholipidosis is not caused by compound U-18666A. In certain instances, the patient's drug-induced phospholipidosis is caused by administration of ABT-770, AC-3579, amantadine, ambroxol, amikacin, amiodarone, amitryptilline, AY-9944, azithromycin, benzamide, boxidine, bromhexine, chlorocyclizine, chloroquine, chlorphentermine, chlorpromazine, citalopram, cloforex, clomipramine, clozapine, compound 200-15, cyclizine, DMP 777, erythromycin, fenfluramine, fluoxetine, fluvoxamine, gentamicin, hydroxyzine, IA-3, imipramine, iprindole, LY281389, maprotiline, meclizine, mepacrine, NE-10064, netilmicin, norchlorcyclizine, noxiptilin, perhexyline, phentermine, PNU-177864, promaxine, promethazine, propanolol, RMI 10.393, sertraline, tamoxifen, thioridazine, tilarone, tobramycin, trimipramine, triparanol, triperennamine, trospectomycin, zimelidine, 1-chloroamitryptiline, or 4,4′-diethylaminoethoxyhexestrol. In certain instances, the patient's drug-induced phospholipidosis is caused by administration of amiodarone, perhexyline, azithromycin, fluoxetine, imipramine, chlorocyclizine, tamoxifen, or gentamicin.

Another aspect of the invention relates to a method comprising administering to a patient in need thereof a therapeutically effective-amount of a first therapeutic agent and a therapeutically effective-amount of a second therapeutic agent; wherein said first therapeutic agent is a compound of any one of formulae I-IX described herein; and said second therapeutic agent is an anorexic, anti-anginal, antiarrhythmic, antibiotic, anti-cancer agent, antidepressant, anti-estrogen agent, antihistaminic agent, antilipemic agent, antimalarial, antinauseant, antipsychotic agent, antithrombotic agent, antiviral agent, cholesterol synthesis inhibitor, diazepine atypical antipsychotic, histamine Hi-blocker, matrix metalloproteinase inhibitor, neutrophil elastase inhibitor, schistosomicidal agent, secretolytic agent, selective serotonin reuptake inhibitor, or tranquilizer that causes drug-induced phospholipidosis. In certain instances, said second therapeutic agent is an antibiotic, anti-arrythmic, antidepressant, histamine Hi-blocker, or anticancer agent that causes drug-induced phospholipidosis.

Another aspect of the invention relates to a method of treating a mammalian cell suffering from drug-induced phospholipidosis comprising administering to said mammalian cell a therapeutically effective-amount of a compound of any one of formulae I-IX described herein. In certain instances, the drug-induced phospholipidosis was not caused by U-18666A. Another aspect of the invention relates to a method of treating a mammalian cell suffering from drug-induced phospholipidosis comprising administering to said mammalian cell a therapeutically effective-amount of a compound of any one of formulae I-IX described herein, wherein the drug-induced phospholipidosis was caused, at least in part, by ABT-770, AC-3579, amantadine, ambroxol, amikacin, amiodarone, amitryptilline, AY-9944, azithromycin, benzamide, boxidine, bromhexine, chlorocyclizine, chloroquine, chlorphentermine, chlorpromazine, citalopram, cloforex, clomipramine, clozapine, compound 200-15, cyclizine, DMP 777, erythromycin, fenfluramine, fluoxetine, fluvoxamine, gentamicin, hydroxyzine, IA-3, imipramine, iprindole, LY281389, maprotiline, meclizine, mepacrine, NE-10064, netilmicin, norchlorcyclizine, noxiptilin, perhexyline, phentermine, PNU-177864, promaxine, promethazine, propanolol, RMI 10.393, sertraline, tamoxifen, thioridazine, tilarone, tobramycin, trimipramine, triparanol, triperennamine, trospectomycin, zimelidine, 1-chloroamitryptiline, or 4,4′-diethylaminoethoxyhexestrol.

### Preparation of Compounds of the Invention

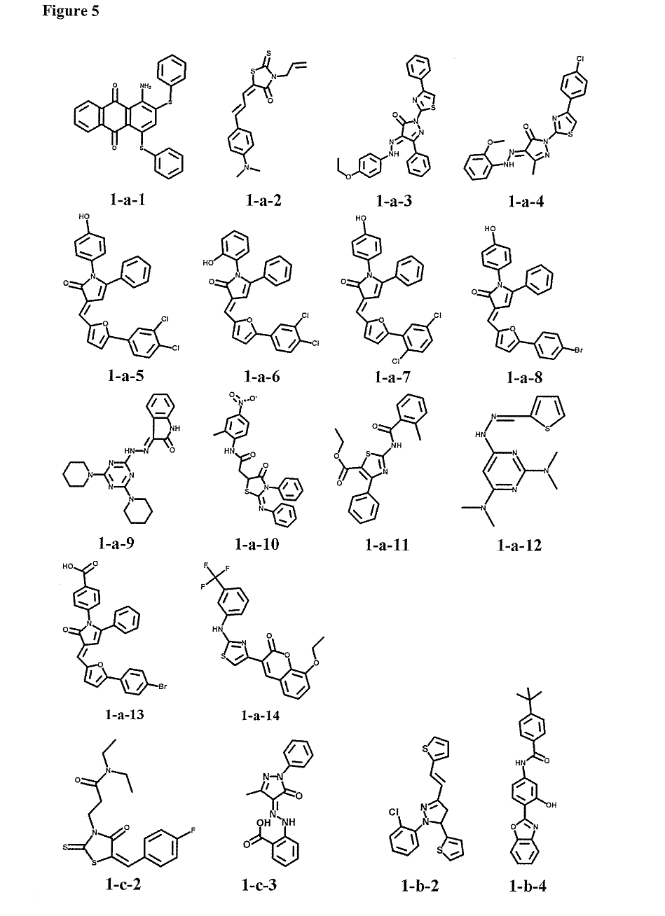

The compounds depicted in FIGS. 5 and 8 are commercially available. The compounds represented by formulae I-XXXIX can be prepared from the compounds depicted in FIGS. 5 and 8, or from other commercially-available compounds using synthetic procedures known in the art. See, for example, J. March, Advanced Organic Chemistry, McGraw Hill Book Company, New York, (1992, 4^{th} edition); Carey, F. A. and Sundberg, R. J. Advanced Organic Chemistry Part B: Reactions and Synthesis, 3^{rd} Ed.; Plenum Press: New York, 1990; and Organic Chemistry 2^{nd} Ed. Ed. Bruice, P. Y. New Jersey: Prentice Hall, 1998. Representative synthetic procedures are also described below.

A large number of compounds can be prepared by installing new functional groups or modifying existing functional groups located on the aromatic rings of the compounds depicted in FIGS. 5 and 8. For example, classical functional group manipulations include installation of bromine by treating an aromatic compound with Br₂ in the presence of FeBr₃, installation of an acyl group by treating the aromatic compound with an acyl chloride in the presence of FeBr₃, treating a nitro-aromatic compound with SnCl₂ in HCl to give an amino-aromatic compound. Other functional group manipulations include reduction of aromatic groups with Na/NH₃ to give a cycloalkene or cycloalkyl compound depending on the reaction conditions. For example, reduction of 1-a-13 using Na/NH₃ would give the cyclohexene derivative selectively due to the effect of the carboxylic acid. See Scheme 1. The cyclohexene intermediate could be further reduced to a cyclohexyl derivative. Alternatively, the cyclohexene intermediate could be treated with an oxidizing agent to form an epoxide. The carboxylic acid group may also be converted to an ester by reaction with an alcohol, such as methanol or benzyl alcohol, in the presence of DCC.

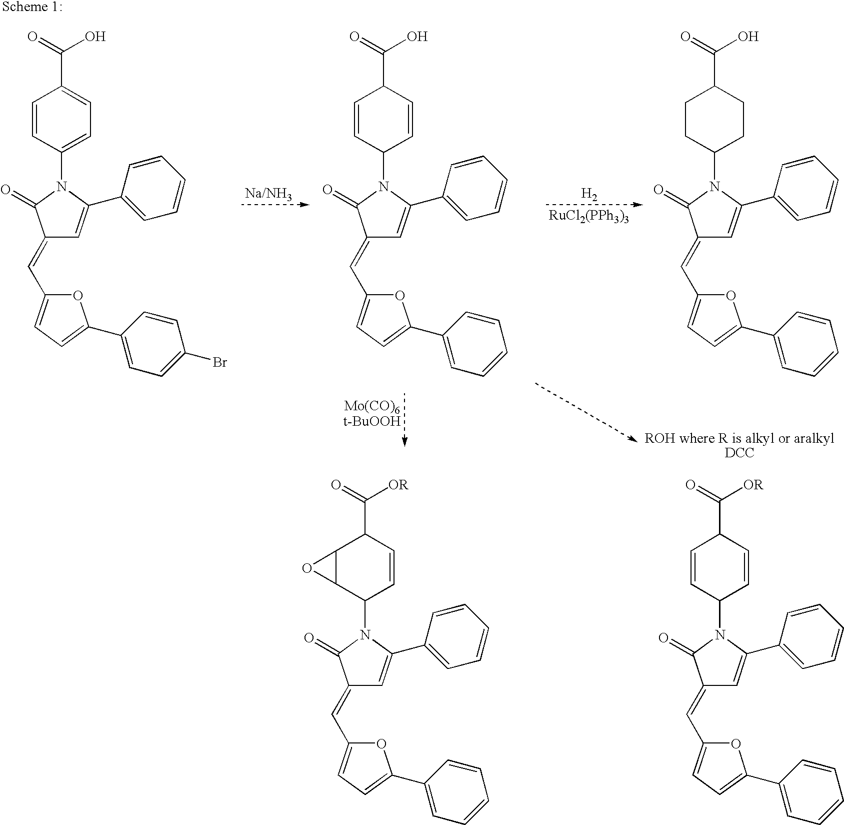

As illustrated in Scheme 2, a large variety of compounds may be prepared from the intermediate lactam-derivative using palladium-coupling techniques. Palladium-coupling reactions are advantageous because they often proceed with high yield and are tollerant of a wide variety of functional groups. Furthermore, a substantial number of organoboranes are known and/or commerically available. Also, a large number of aromatic halides and alkenyl halides are commerical available which can be readily converted to the organoborane starting material for the coupling reaction.

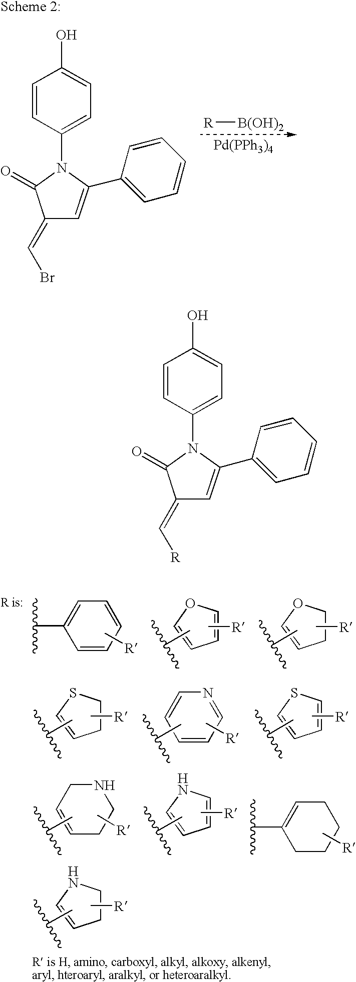

A wide variety of triazine compounds can be prepared using palladium coupling reactions. As illustrated in Scheme 3, reaction of the commerically available triazine with aryl/heteroaryl bromides or iodides could be used to prepare a number of derivatives, each of which could be converted to other compounds using the aromatic functional group manipulations described above. Notably, palladium coupling of aryl/heteroaryl bromides or iodides could also be used to prepare a variety of triazinyl hydrazones as shown in Scheme 4.

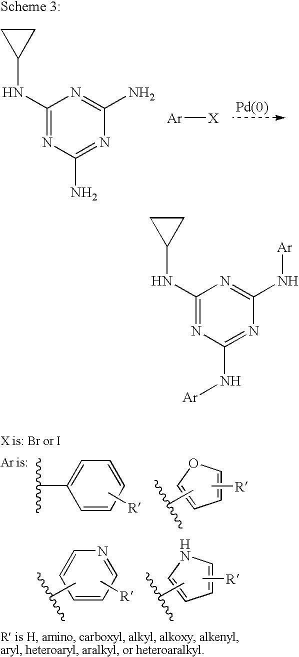

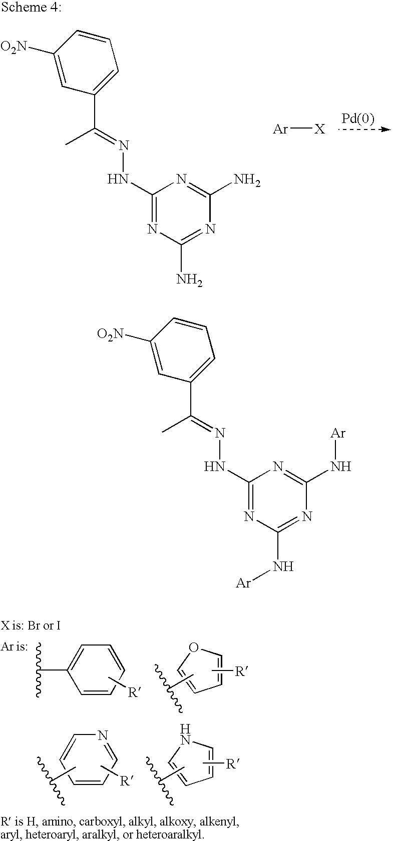

Methods of the Invention

One aspect of the present invention relates to a method of treating a patient suffering from a disorder characterized by cellular accumulation of cholesterol, comprising the step of:

administering to a patient in need thereof a therapeutically effective amount of a compound of any one of formulae I-IX, wherein formula I is represented by:

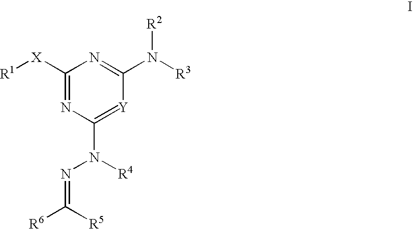

wherein,

X is O or —N(R⁷)—;

Y is N or —C(R⁸)—;

R¹ and R² represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, heteroaralkyl, —(CR⁹═CR⁹)ₙ-aryl, or —(CR⁹═CR⁹)ₙ-heteroaryl;

R⁶ is H or alkyl; or R⁵ and R⁶ taken together form a optionally substituted monocyclic or bicyclic ring having 1 or 2 heteroatoms selected from the group consisting of O, N, and S;

R⁷ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁷ taken together form a 3-8 member ring optionally substituted with one or more of alkyl, halogen, hydroxy, alkoxy, or amino;

R⁸ and R⁹ represent independently for each occurrence H or alkyl; and

n is 1 or 2;

formula II is represented by:

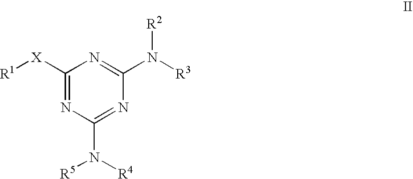

wherein,

X is O or —N(R⁶)—;

R¹ and R² represent independently cycloalkyl, heterocycloalkyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, or heteroaralkyl;

R⁶ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁶ taken together form a 3-8 member ring optionally substituted with one or more of alkyl, halogen, hydroxy, alkoxy, or amino;

formula III is represented by:

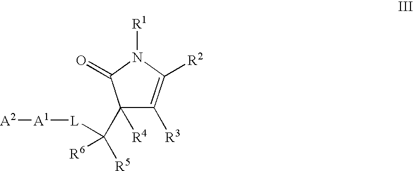

wherein,

R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);

R² is cycloalkyl, heterocycloalkyl, cycloalkenyl, heterocycloalkenyl, aryl, heteroaryl, aralkyl, heteroaralkyl, or alkyl;

R³ is hydrogen, alkyl, —CO₂R⁸, or —C(O)N(R⁷)(R⁸);

R⁴ and R⁵ represent independently H or alkyl; or R⁴ and R⁵ taken together form a bond;

R⁶ and R⁷ represent independently for each occurrence H or alkyl;

R⁸ represents independently for each occurrence alkyl, cycloalkyl, aryl, heteroaryl, aralkyl, or heteroaralkyl;

L is a bond, —C(R⁷)₂—, or —(CR⁷═CR⁷)—; and

A¹ and A² represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;

formula IV is represented by:

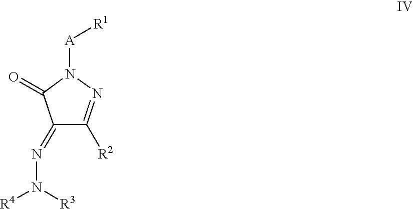

wherein,

A is cycloalkenyl, heterocycloalkenyl, aryl, heteroaryl, aralkyl, or heteroaralkyl;

R¹ is cycloalkenyl, heterocycloalkenyl, aryl, heteroaryl, aralkyl, heteroaralkyl, —(CR³═CR³)-aryl, or —(CR³═CR³)-heteroaryl;

R² is alkyl, cycloalkyl, heterocycloalkyl, cycloalkenyl, heterocycloalkenyl, aryl, heteroaryl, aralkyl, or heteroaralkyl;

R³ represents independently for each occurrence H or alkyl; and

R⁴ is cycloalkyl, heterocycloalkyl, cycloalkenyl, heterocycloalkenyl, aryl, heteroaryl, aralkyl, or heteroaralkyl;

formula V is represented by:

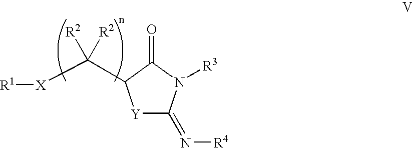

wherein,

X is O, —N(R⁵)—, —N(R⁵)C(O)—, —C(O)N(R⁵)—, —OC(O)—, —CO₂—, or —N(R⁵)CO₂—;

Y is O, S, or —N(R⁵)—;

R¹ is cycloalkenyl, heterocycloalkenyl, aryl, heteroaryl, aralkyl, or heteroaralkyl;

R² represents independently for each occurrence H or alkyl, or two R² taken together form ═O;

R³ and R⁴ represent independently cycloalkyl, heterocycloalkyl, cycloalkenyl, heterocycloalkenyl, aryl, heteroaryl, aralkyl, or heteroaralkyl;

R⁵ represents independently for each occurrence H, alkyl, aryl, or aralkyl; and

n is 1, 2, 3, 4, or 5;

formula VI is represented by:

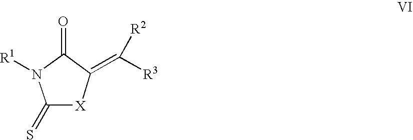

wherein,

X is O, S, or —N(R⁴)—;

R¹ is cycloalkyl, heterocycloalkyl, cycloalkenyl, heterocycloalkenyl, aryl, heteroaryl, aralkyl, heteroaralkyl, or —(C(R⁵)₂)ₙ—(CR⁵═C(R⁵)₂);

R² is cycloalkenyl, heterocycloalkenyl, aryl, heteroaryl, aralkyl, heteroaralkyl, —(CR⁵═CR⁵)-aryl, or —(CR⁵═CR⁵)-heteroaryl;

R³ is H, alkyl, alkenyl, aryl, or heteroaryl; or R² and R³ taken together form an optionally substituted monocyclic or bicyclic ring having 0, 1, or 2 heteroatoms selected from the group consisting of O, N, and S;

R⁴ and R⁵ represent independently for each occurrence H, alkyl, aryl, or aralkyl; and

n is 1, 2, 3, 4, or 5;

formula VII is represented by:

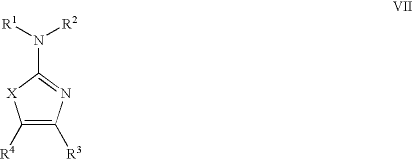

wherein,

X is O or S;

R¹ is cycloalkenyl, heterocycloalkenyl, aryl, heteroaryl, aralkyl, heteroaralkyl, or —C(O)R⁵;

R² is H or alkyl;

R³ is cycloalkenyl, heterocycloalkenyl, aryl, heteroaryl, aralkyl, heteroaralkyl, or an optionally substituted bicyclic ring having 1 or 2 heteroatoms selected form the group consisting of O, N, and S;

R⁴ is H, alkyl, —CO₂R⁶, or —C(O)N(R⁶)₂;

R⁵ is cycloalkenyl, heterocycloalkenyl, heteroaryl, aralkyl, or heteroaralkyl; or R⁵ is an aryl group optionally substituted with one or more of alkyl, halogen, —OR⁶, —N(R⁶)₂, —CO₂R⁶, C(O)N(R⁶)₂, cyano, or nitro; and

R⁶ represents independently for each occurrence H, alkyl, cycloalkyl, heterocycloalkyl, aryl, heteroaryl, aralkyl, or heteroaralkyl;

formula VIII is represented by:

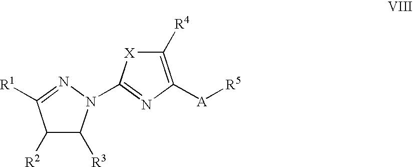

wherein,

X is O or S;

R¹, R³, and A represent independently cycloalkenyl, heterocycloalkenyl, aryl, heteroaryl, aralkyl, or heteroaralkyl;

R² and R⁴ represent independently H or alkyl;

R⁵ is an optionally substituted monocyclic or bicyclic ring having 1, 2, or 3 heteroatoms selected from the group consisting of O, N, and S; and

formula IX is represented by:

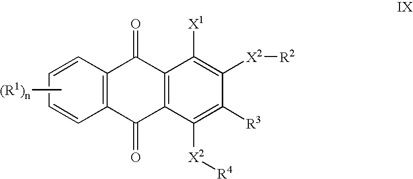

wherein,

X¹ is —OR⁵, —SR⁵, or —N(R⁵)₂;

X represents independently for each occurrence O, S, or —N(R⁵)—;

R¹ represents independently for each occurrence alkyl, halogen, nitro, cyano, alkoxyl, alkenyl, alkynyl, cycloalkenyl, heterocycloalkenyl, aryl, heteroaryl, aralkyl, or heteroaralkyl, —N(R⁵)₂, —OH, —C(O)R⁶, —CO₂R⁵, or C(O)N(R⁵)₂;

R² and R⁴ represent independently cycloalkenyl, heterocycloalkenyl, aryl, heteroaryl, aralkyl, or heteroaralkyl;

R³ is H, alkyl, or halogen;

R⁵ represents independently for each occurrence H, alkyl, aryl, heteroaryl, aralkyl, or heteroaralkyl;

R⁶ represents independently for each occurrence alkyl, cycloalkyl, heterocycloalkyl, aryl, heteroaryl, aralkyl, or heteroaralkyl; and

n is 0, 1, 2, 3, or 4.

In certain embodiments, the present invention relates to the aforementioned method, wherein the disorder is Niemann-Pick disease type C.

In certain embodiments, the present invention relates to the aforementioned method, wherein the disorder is atherosclerosis.

In certain embodiments, the present invention relates to the aforementioned method, wherein the disorder is a Lysosomal storage disorder arising from a defect in sphingolipid or glycosphingolipid metabolism.

In certain embodiments, the present invention relates to the aforementioned method, wherein said compound is a compound of formula I.

In certain embodiments, the present invention relates to the aforementioned method, wherein said compound is a compound of formula I, X is O or —N(R⁷)—; Y is N; R¹ and R² represent independently alkyl, haloalkyl, or aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is —N(R⁷)—; Y is N; R¹ and R² are aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is O or —N(R⁷)—; Y is —C(R⁸)—; R¹ and R² represent independently alkyl, heteroalkyl, or haloalkyl; R³ is hydrogen, alkyl, heteroalkyl, or haloalkyl; R⁴ is hydrogen; R⁵ is heteroaryl; R⁶ is H or alkyl; R⁷ is hydrogen, alkyl, heteroalkyl, or haloalkyl; and R⁸ is H or alkyl.

In certain embodiments, the present invention relates to the aforementioned method, wherein said compound is a compound of formula II.

In certain embodiments, the present invention relates to the aforementioned method, wherein said compound is a compound of formula II, X is —N(R⁶)—; R¹, R² and R⁵ represent independently aryl or heteroaryl; and R³, R⁴, and R⁶ represent independently hydrogen or alkyl. In certain embodiments, the present invention relates to the aforementioned method, wherein said compound is a compound of formula III.

In certain embodiments, the present invention relates to the aforementioned method, wherein said compound is a compound of formula III; R¹, R², A¹, and A² represent independently aryl or heteroaryl; R³ is hydrogen or alkyl; R⁶ is H or alkyl; and L is a bond.

In certain embodiments, the present invention relates to the aforementioned method, wherein said compound is a compound of formula III, R¹ is —(C(R⁷)₂)ₙ—(CR⁷═C(R⁷)₂); R² is alkyl;

R³ is alkyl, —CO₂R⁸, or —C(O)N(R⁷)(R⁸); R⁴ and R⁵ represent independently H or alkyl; or R⁴ and R⁵ taken together form a bond; R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

In certain embodiments, the present invention relates to the aforementioned method, wherein said compound is a compound of formula III, R¹ comprises a carboxylic acid group; R¹ is a carboxylic acid substituted aryl; R¹ is a carboxylic acid substituted phenyl; and/or R¹ is a para-substituted carboxylic acid phenyl.

In certain embodiments, the present invention relates to the aforementioned method, wherein said compound is a compound of formula IV.

In certain embodiments, the present invention relates to the aforementioned method, wherein said compound is a compound of formula IV; A, R¹, and R⁴ represent independently aryl or heteroaryl; R¹ is cycloalkenyl, heterocycloalkenyl, aryl, heteroaryl, aralkyl, heteroaralkyl, —(CR³═CR³)-aryl, or —(CR³═CR³)-heteroaryl; R² is alkyl or aryl; and R³ represents independently for each occurrence H or alkyl.

In certain embodiments, the present invention relates to the aforementioned method, wherein said compound is a compound of formula V.

In certain embodiments, the present invention relates to the aforementioned method, wherein said compound is a compound of formula V; X is —C(O)N(R⁵)— or —CO₂—; Y is O or S; R¹, R³, and R⁴ represent independently aryl or heteroaryl; R² represents independently for each occurrence H or alkyl, or two R² taken together form ═O; R³ and R⁴ represent independently cycloalkyl, heterocycloalkyl, cycloalkenyl, heterocycloalkenyl, aryl, heteroaryl, aralkyl, or heteroaralkyl; R⁵ represents independently for each occurrence H, alkyl, aryl, or aralkyl; and n is 1 or 2;

In certain embodiments, the present invention relates to the aforementioned method, wherein said compound is a compound of formula VI.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is aryl, heteroaryl, —(CR⁵═CR⁵)-aryl or —(CR⁵═CR⁵)-heteroaryl; R³ is H or alkyl; P R⁵ represents independently for each occurrence H, alkyl, aryl, or aralkyl; and n is 1 or 2.

In certain embodiments, the present invention relates to the aforementioned method, wherein said compound is a compound of formula VI, X is S, R¹ is aryl, R² is aryl, and R³ is H or alkyl.

In certain embodiments, the present invention relates to the aforementioned method, wherein said compound is a compound of formula VI, X is S, R¹ is alkoxy-substituted phenyl, R² is dialkylamino-substituted phenyl, and R³ is H.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is —(CR⁵═CR⁵)-aryl or —(CR⁵═CR⁵)-heteroaryl; R³ is H or alkyl; or R² and R³ taken together form an optionally substituted monocyclic or bicyclic ring having 0, 1, or 2 heteroatoms selected from the group consisting of O, N, and S; R⁵ represents independently for each occurrence H, alkyl, aryl, or aralkyl; and n is 1 or 2.

In certain embodiments, the present invention relates to the aforementioned method, wherein said compound is a compound of formula VII.

In certain embodiments, the present invention relates to the aforementioned method, wherein said compound is a compound of formula VII; X is O or S; R¹ is aryl, heteroaryl, or —C(O)R⁵; R² is H or alkyl; R³ is aryl, heteroaryl, or an optionally substituted bicyclic ring having 1 or 2 heteroatoms selected form the group consisting of O, N, and S; R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; and R⁶ represents independently for each occurrence H, alkyl, aryl, heteroaryl, aralkyl, or heteroaralkyl.

In certain embodiments, the present invention relates to the aforementioned method, wherein said compound is a compound of formula VII; X is O or S; R¹ is aryl, heteroaryl, or —C(O)R⁵; R² is H or alkyl; R³ is represented

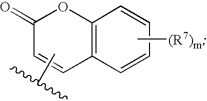

by R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; R⁶ represents independently for each occurrence H, alkyl, aryl, heteroaryl, aralkyl, or heteroaralkyl; m is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula VIII.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; and R⁵ is an optionally substituted bicyclic ring having 1, 2, or 3 heteroatoms selected from the group consisting of O, N, and S.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; R⁵ is represented by

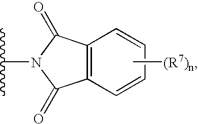

wherein n is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula IX;

In certain embodiments, the present invention relates to the aforementioned method, wherein said compound is a compound of formula IX; X¹ is —N(R⁵)₂; X² represents independently for each occurrence O, or S; R¹ represents independently for each occurrence alkyl, halogen, nitro, cyano, alkoxyl, —N(R⁵)₂, —OH, —C(O)R⁶, —CO₂R⁵, or C(O)N(R⁵)₂; R² and R⁴ represent independently aryl, heteroaryl, aralkyl, or heteroaralkyl; R³ is H, alkyl, or halogen; R⁵ represents independently for each occurrence H, alkyl, aryl, heteroaryl, aralkyl, or heteroaralkyl; R⁶ represents independently for each occurrence alkyl, cycloalkyl, heterocycloalkyl, aryl, heteroaryl, aralkyl, or heteroaralkyl; and n is 0, 1, 2, 3, or 4.

In certain embodiments, the present invention relates to the aforementioned method, wherein said compound is

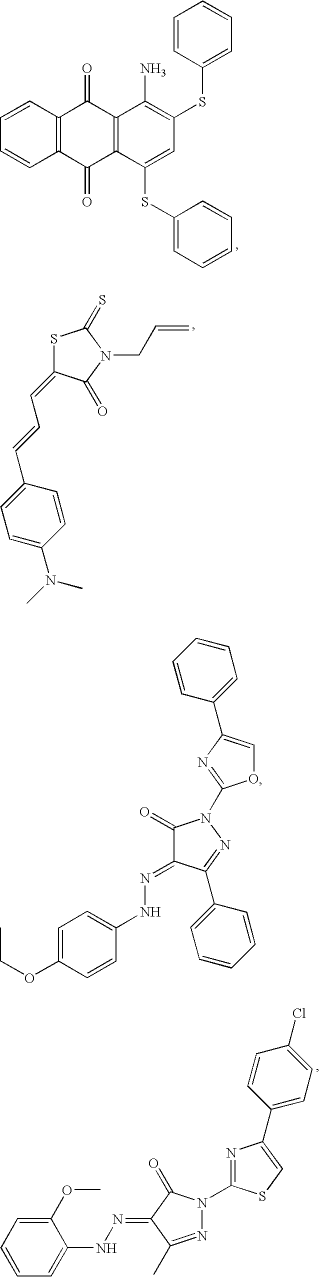

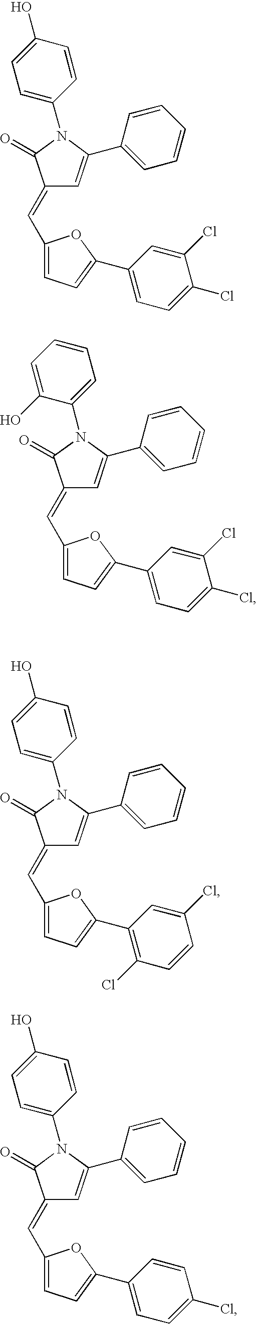

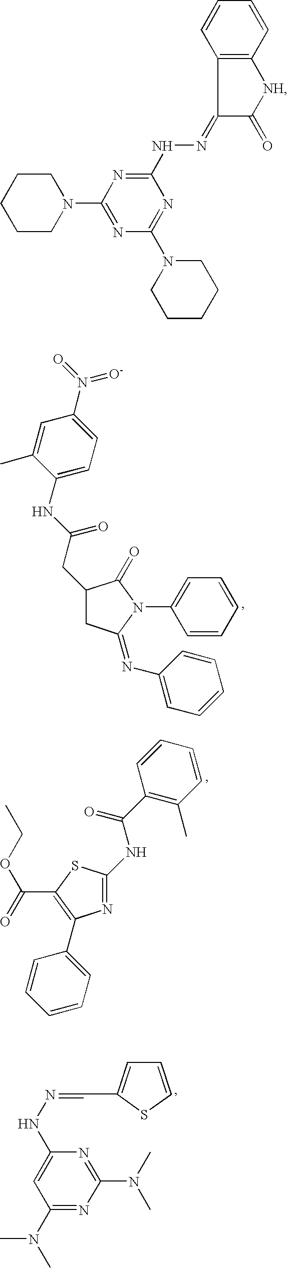

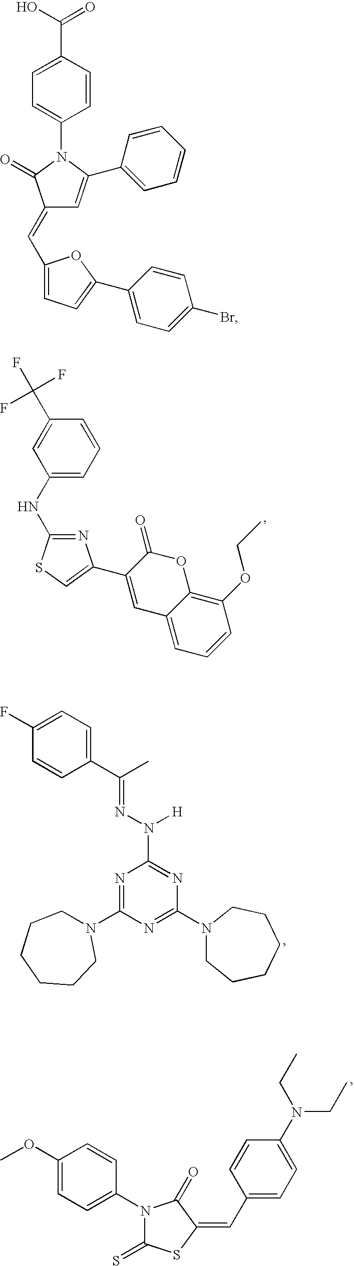

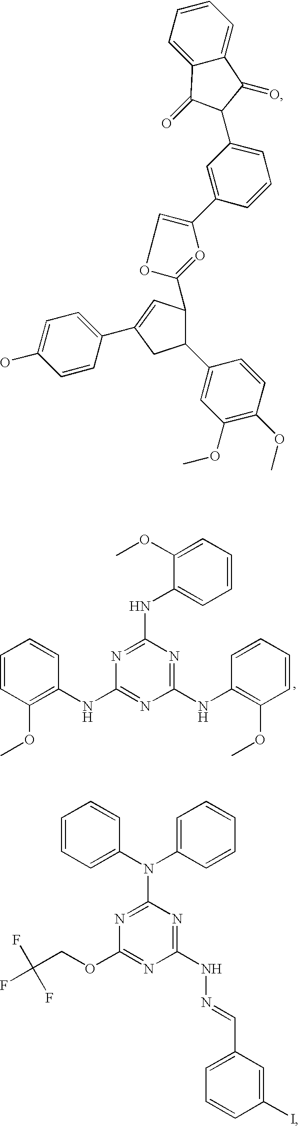

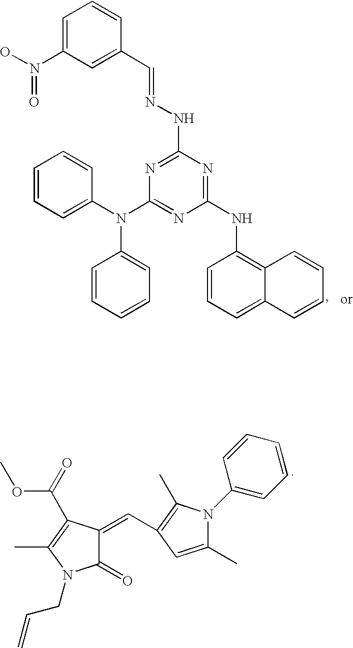

In certain embodiments, the present invention relates to the aforementioned method, wherein said compound is a compound of any one of formulae X-XXXIX as described below.

Another aspect of the present invention relates to a method of reducing the amount of cholesterol in a cell, comprising the step of:

exposing a mammalian cell to a compound of any one of formulae I-IX, wherein formula I is represented by:

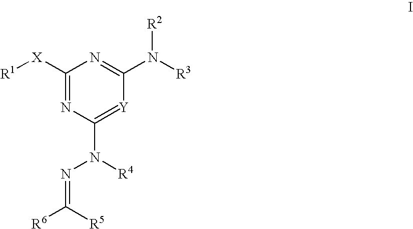

wherein,

X is O or —N(R⁷)—;

Y is N or —C(R⁸)—;

R¹ and R² represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, heteroaralkyl, —(CR⁹═CR⁹)ₙ-aryl, or —(CR⁹═CR⁹)ₙ-heteroaryl;

R⁶ is H or alkyl; or R⁵ and R⁶ taken together form a optionally substituted monocyclic or bicyclic ring having 1 or 2 heteroatoms selected from the group consisting of O, N, and S;

R⁷ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁷ taken together form a 3-8 member ring optionally substituted with one or more of alkyl, halogen, hydroxy, alkoxy, or amino;

R⁸ and R⁹ represent independently for each occurrence H or alkyl; and

n is 1 or 2;

formula II is represented by:

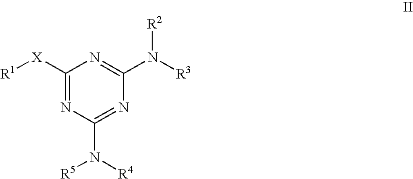

wherein,

X is O or —N(R⁶)—;

R¹ and R² represent independently cycloalkyl, heterocycloalkyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, or heteroaralkyl;

R⁶ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁶ taken together form a 3-8 member ring optionally substituted with one or more of alkyl, halogen, hydroxy, alkoxy, or amino;

formula III is represented by:

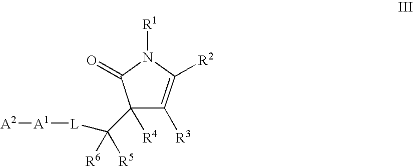

wherein,

R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);

R² is cycloalkyl, heterocycloalkyl, cycloalkenyl, heterocycloalkenyl, aryl, heteroaryl, aralkyl, heteroaralkyl, or alkyl;

R³ is hydrogen, alkyl, —CO₂R⁸, or —C(O)N(R⁷)(R⁸);

R⁴ and R⁵ represent independently H or alkyl; or R⁴ and R⁵ taken together form a bond;

R⁶ and R⁷ represent independently for each occurrence H or alkyl;

R⁸ represents independently for each occurrence alkyl, cycloalkyl, aryl, heteroaryl, aralkyl, or heteroaralkyl;

L is a bond, —C(R⁷)₂—, or —(CR⁷═CR⁷)—; and

A¹ and A² represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;

formula IV is represented by:

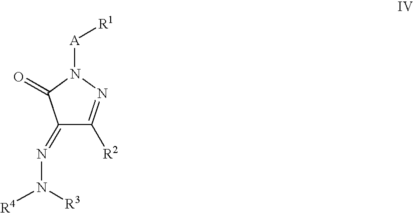

wherein,

A is cycloalkenyl, heterocycloalkenyl, aryl, heteroaryl, aralkyl, or heteroaralkyl;

R¹ is cycloalkenyl, heterocycloalkenyl, aryl, heteroaryl, aralkyl, heteroaralkyl, —(CR³═CR³)-aryl, or —(CR³═CR³)-heteroaryl;

R² is alkyl, cycloalkyl, heterocycloalkyl, cycloalkenyl, heterocycloalkenyl, aryl, heteroaryl, aralkyl, or heteroaralkyl;

R³ represents independently for each occurrence H or alkyl; and

R⁴ is cycloalkyl, heterocycloalkyl, cycloalkenyl, heterocycloalkenyl, aryl, heteroaryl, aralkyl, or heteroaralkyl;

formula V is represented by:

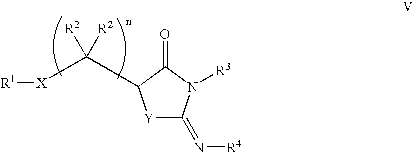

wherein,

X is O, —N(R⁵)—, —N(R⁵)C(O)—, —C(O)N(R⁵)—, —OC(O)—, —CO₂—, or —N(R⁵)CO₂—;

Y is O, S, or —N(R⁵)—;

R¹ is cycloalkenyl, heterocycloalkenyl, aryl, heteroaryl, aralkyl, or heteroaralkyl;

R² represents independently for each occurrence H or alkyl, or two R² taken together form ═O;

R³ and R⁴ represent independently cycloalkyl, heterocycloalkyl, cycloalkenyl, heterocycloalkenyl, aryl, heteroaryl, aralkyl, or heteroaralkyl;

R⁵ represents independently for each occurrence H, alkyl, aryl, or aralkyl; and

n is 1, 2, 3, 4, or 5;

formula VI is represented by:

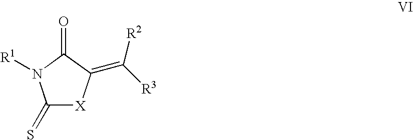

wherein,

X is O, S, or —N(R⁴)—;

R¹ is cycloalkyl, heterocycloalkyl, cycloalkenyl, heterocycloalkenyl, aryl, heteroaryl, aralkyl, heteroaralkyl, or —(C(R⁵)₂)ₙ—(CR⁵═C(R⁵)₂);

R² is cycloalkenyl, heterocycloalkenyl, aryl, heteroaryl, aralkyl, heteroaralkyl, —(CR⁵═CR⁵)-aryl, or —(CR⁵═CR⁵)-heteroaryl;

R³ is H, alkyl, alkenyl, aryl, or heteroaryl; or R² and R³ taken together form an optionally substituted monocyclic or bicyclic ring having 0, 1, or 2 heteroatoms selected from the group consisting of O, N, and S;

R⁴ and R⁵ represent independently for each occurrence H, alkyl, aryl, or aralkyl; and

n is 1, 2, 3, 4, or 5;

formula VII is represented by:

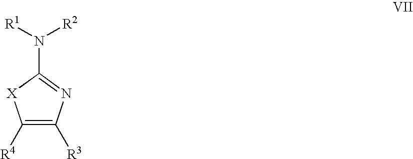

wherein,

X is O or S;

R¹ is cycloalkenyl, heterocycloalkenyl, aryl, heteroaryl, aralkyl, heteroaralkyl, or —C(O)R⁵;

R² is H or alkyl;

R¹ is cycloalkenyl, heterocycloalkenyl, aryl, heteroaryl, aralkyl, heteroaralkyl, or an optionally substituted bicyclic ring having 1 or 2 heteroatoms selected form the group consisting of O, N, and S;

R⁴ is H, alkyl, —CO₂R⁶, or —C(O)N(R⁶)₂;

R⁵ is cycloalkenyl, heterocycloalkenyl, heteroaryl, aralkyl, or heteroaralkyl; or R⁵ is an aryl group optionally substituted with one or more of alkyl, halogen, —OR⁶, —N(R⁶)₂, —CO₂R⁶, C(O)N(R⁶)₂, cyano, or nitro; and

R⁶ represents independently for each occurrence H, alkyl, cycloalkyl, heterocycloalkyl, aryl, heteroaryl, aralkyl, or heteroaralkyl;

formula VIII is represented by:

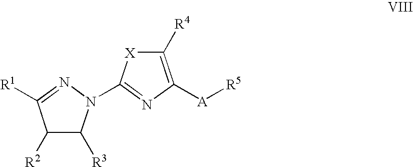

wherein,

X is O or S;

R¹, R³, and A represent independently cycloalkenyl, heterocycloalkenyl, aryl, heteroaryl, aralkyl, or heteroaralkyl;

R² and R⁴ represent independently H or alkyl;

R⁵ is an optionally substituted monocyclic or bicyclic ring having 1, 2, or 3 heteroatoms selected from the group consisting of O, N, and S; and

formula IX is represented by:

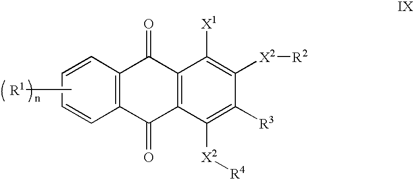

wherein,

X¹ is —OR⁵, —SR⁵, or —N(R⁵)₂;

X² represents independently for each occurrence O, S, or —N(R⁵)—;

R¹ represents independently for each occurrence alkyl, halogen, nitro, cyano, alkoxyl, alkenyl, alkynyl, cycloalkenyl, heterocycloalkenyl, aryl, heteroaryl, aralkyl, or heteroaralkyl, —N(R⁵)₂, —OH, —C(O)R⁶, —CO₂R⁵, or C(O)N(R⁵)₂;

R² and R⁴ represent independently cycloalkenyl, heterocycloalkenyl, aryl, heteroaryl, aralkyl, or heteroaralkyl;

R³ is H, alkyl, or halogen;

R⁵ represents independently for each occurrence H, alkyl, aryl, heteroaryl, aralkyl, or heteroaralkyl;

R⁶ represents independently for each occurrence alkyl, cycloalkyl, heterocycloalkyl, aryl, heteroaryl, aralkyl, or heteroaralkyl; and

n is 0, 1, 2, 3, or 4.

In certain embodiments, the present invention relates to the aforementioned method, wherein said compound reduces the amount of cholesterol in said cell by increasing cholesterol efflux from said cell.

In certain embodiments, the present invention relates to the aforementioned method, wherein said compound reduces the amount of cholesterol in said cell by inhibiting cholesterol uptake by said cell.

In certain embodiments, the present invention relates to the aforementioned method, wherein said compound reduces the amount of cholesterol by inhibiting cholesterol synthesis by said cell.

In certain embodiments, the present invention relates to the aforementioned method, wherein said compound reduces the amount of cholesterol in said cell by promoting esterification of cholesterol in said cell.

In certain embodiments, the present invention relates to the aforementioned method, wherein said cell is a human cell.

In certain embodiments, the present invention relates to the aforementioned method, wherein said cell has a Niemann-Pick Type C defect.

In certain embodiments, the present invention relates to the aforementioned method, wherein said compound is a compound of formula I.

In certain embodiments, the present invention relates to the aforementioned method, wherein said compound is a compound of formula I, X is O or —N(R⁷)—; Y is N; R¹ and R² represent independently alkyl, haloalkyl, or aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is —N(R⁷)—; Y is N; R¹ and R² are aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is O or —N(R⁷)—; Y is —C(R⁸)—; R¹ and R² represent independently alkyl, heteroalkyl, or haloalkyl; R³ is hydrogen, alkyl, heteroalkyl, or haloalkyl; R⁴ is hydrogen; R⁵ is heteroaryl; R⁶ is H or alkyl; R⁷ is hydrogen, alkyl, heteroalkyl, or haloalkyl; and R⁸ is H or alkyl.

In certain embodiments, the present invention relates to the aforementioned method, wherein said compound is a compound of formula II.

In certain embodiments, the present invention relates to the aforementioned method, wherein said compound is a compound of formula II, X is —N(R⁶)—; R¹, R² and R⁵ represent independently aryl or heteroaryl; and R³, R⁴, and R⁶ represent independently hydrogen or alkyl.

In certain embodiments, the present invention relates to the aforementioned method, wherein said compound is a compound of formula III.

In certain embodiments, the present invention relates to the aforementioned method, wherein said compound is a compound of formula III; R¹, R², A¹, and A² represent independently aryl or heteroaryl; R³ is hydrogen or alkyl; R⁶ is H or alkyl; and L is a bond.

In certain embodiments, the present invention relates to the aforementioned method, wherein said compound is a compound of formula III, R¹ is —(C(R⁷)₂)ₙ—(CR⁷═C(R⁷)₂); R² is alkyl;

R³ is alkyl, —CO₂R⁸, or —C(O)N(R⁷)(R⁸); R⁴ and R⁵ represent independently H or alkyl; or R⁴ and R⁵ taken together form a bond; R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

In certain embodiments, the present invention relates to the aforementioned method, wherein said compound is a compound of formula III, R¹ comprises a carboxylic acid group; R¹ is a carboxylic acid substituted aryl; R¹ is a carboxylic acid substituted phenyl; and/or R¹ is a para-substituted carboxylic acid phenyl.

In certain embodiments, the present invention relates to the aforementioned method, wherein said compound is a compound of formula IV.

In certain embodiments, the present invention relates to the aforementioned method, wherein said compound is a compound of formula IV; A, R¹, and R⁴ represent independently aryl or heteroaryl; R¹ is cycloalkenyl, heterocycloalkenyl, aryl, heteroaryl, aralkyl, heteroaralkyl, —(CR³═CR³)-aryl, or —(CR³═CR³)-heteroaryl; R² is alkyl or aryl; and R³ represents independently for each occurrence H or alkyl.

In certain embodiments, the present invention relates to the aforementioned method, wherein said compound is a compound of formula V.

In certain embodiments, the present invention relates to the aforementioned method, wherein said compound is a compound of formula V; X is —C(O)N(R⁵)— or —CO₂—; Y is O or S; R¹, R³, and R⁴ represent independently aryl or heteroaryl; R² represents independently for each occurrence H or alkyl, or two R² taken together form ═O; R³ and R⁴ represent independently cycloalkyl, heterocycloalkyl, cycloalkenyl, heterocycloalkenyl, aryl, heteroaryl, aralkyl, or heteroaralkyl; R⁵ represents independently for each occurrence H, alkyl, aryl, or aralkyl; and n is 1 or 2;

In certain embodiments, the present invention relates to the aforementioned method, wherein said compound is a compound of formula VI.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is aryl, heteroaryl, —(CR⁵═CR⁵)-aryl or —(CR⁵═CR⁵)-heteroaryl; R³ is H or alkyl; R⁵ represents independently for each occurrence H, alkyl, aryl, or aralkyl; and n is 1 or 2.

In certain embodiments, the present invention relates to the aforementioned method, wherein said compound is a compound of formula VI, X is S, R¹ is aryl, R² is aryl, and R³ is H or alkyl.

In certain embodiments, the present invention relates to the aforementioned method, wherein said compound is a compound of formula VI, X is S, R¹ is alkoxy-substituted phenyl, R² is dialkylamino-substituted phenyl, and R³ is H.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is —(CR⁵═CR⁵)-aryl or —(CR⁵═CR⁵)-heteroaryl; R³ is H or alkyl; or R² and R³ taken together form an optionally substituted monocyclic or bicyclic ring having 0, 1, or 2 heteroatoms selected from the group consisting of O, N, and S; R⁵ represents independently for each occurrence H, alkyl, aryl, or aralkyl; and n is 1 or 2.

In certain embodiments, the present invention relates to the aforementioned method, wherein said compound is a compound of formula VII.

In certain embodiments, the present invention relates to the aforementioned method, wherein said compound is a compound of formula VII; X is O or S; R⁵ is aryl, heteroaryl, or —C(O)R⁵; R² is H or alkyl; R³ is aryl, heteroaryl, or an optionally substituted bicyclic ring having 1 or 2 heteroatoms selected form the group consisting of O, N, and S; R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; and R⁶ represents independently for each occurrence H, alkyl, aryl, heteroaryl, aralkyl, or heteroaralkyl.

In certain embodiments, the present invention relates to the aforementioned method, wherein said compound is a compound of formula VII; X is O or S; R¹ is aryl, heteroaryl, or —C(O)R⁵; R² is H or alkyl; R³ is represented by

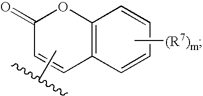

R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; R⁶ represents independently for each occurrence H, alkyl, aryl, heteroaryl, aralkyl, or heteroaralkyl; m is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula VIII.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; and R⁵ is an optionally substituted bicyclic ring having 1, 2, or 3 heteroatoms selected from the group consisting of O, N, and S.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; R⁵ is represented by

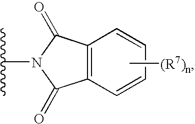

wherein n is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula IX;

In certain embodiments, the present invention relates to the aforementioned method, wherein said compound is a compound of formula IX; X¹ is —N(R⁵)₂; X² represents independently for each occurrence O, or S; R¹ represents independently for each occurrence alkyl, halogen, nitro, cyano, alkoxyl, —N(R⁵)₂, —OH, —C(O)R⁶, —CO₂R⁵, or C(O)N(R⁵)₂; R² and R⁴ represent independently aryl, heteroaryl, aralkyl, or heteroaralkyl; R³ is H, alkyl, or halogen; R⁵ represents independently for each occurrence H, alkyl, aryl, heteroaryl, aralkyl, or heteroaralkyl; R⁶ represents independently for each occurrence alkyl, cycloalkyl, heterocycloalkyl, aryl, heteroaryl, aralkyl, or heteroaralkyl; and n is 0, 1, 2, 3, or 4.

In certain embodiments, the present invention relates to the aforementioned method, wherein said compound is

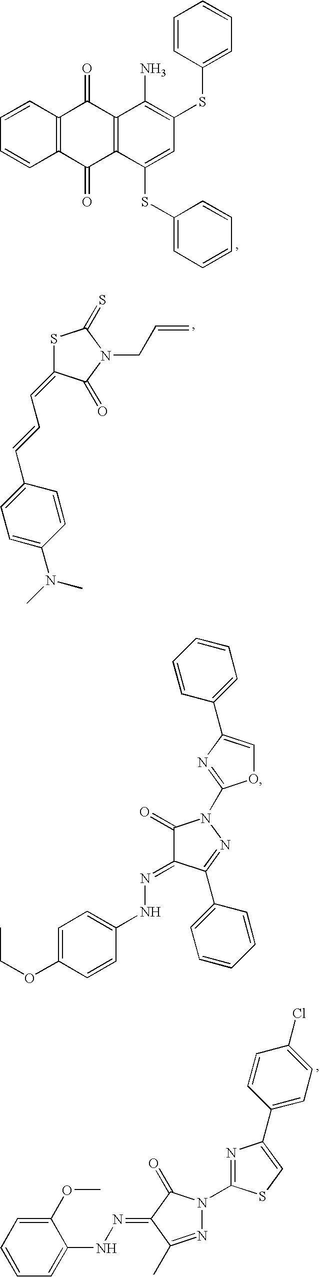

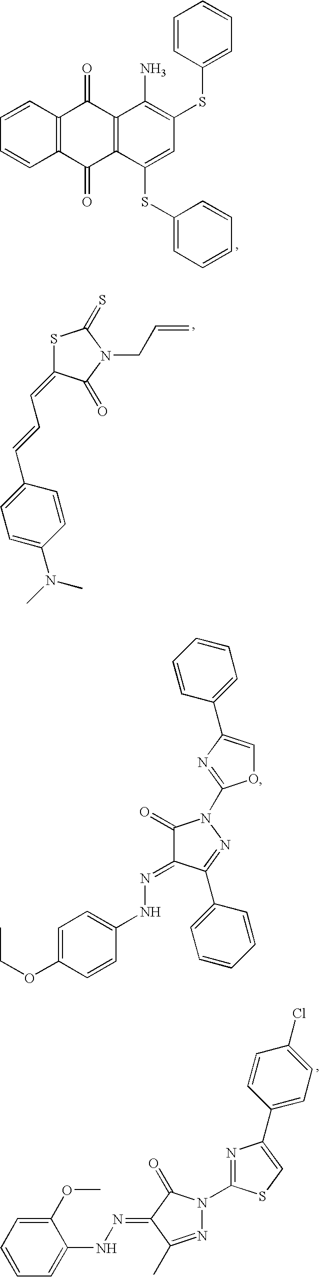

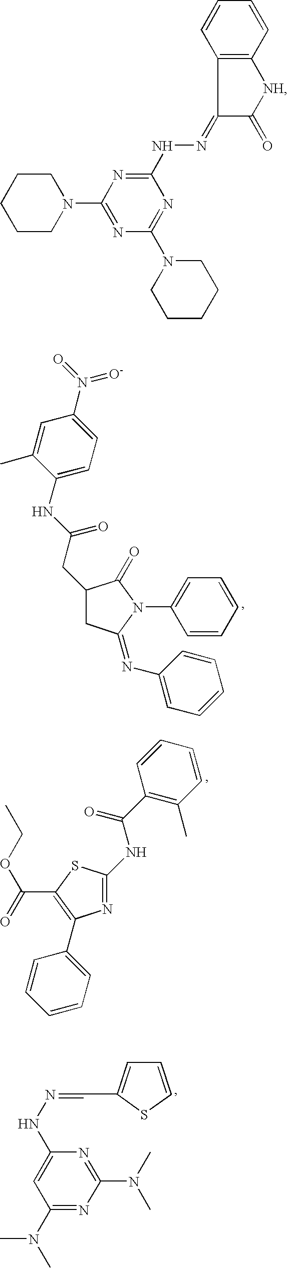

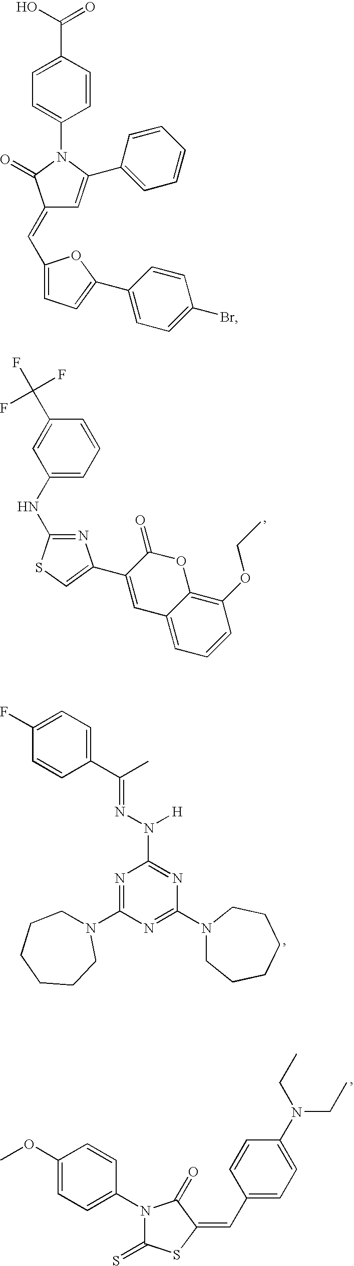

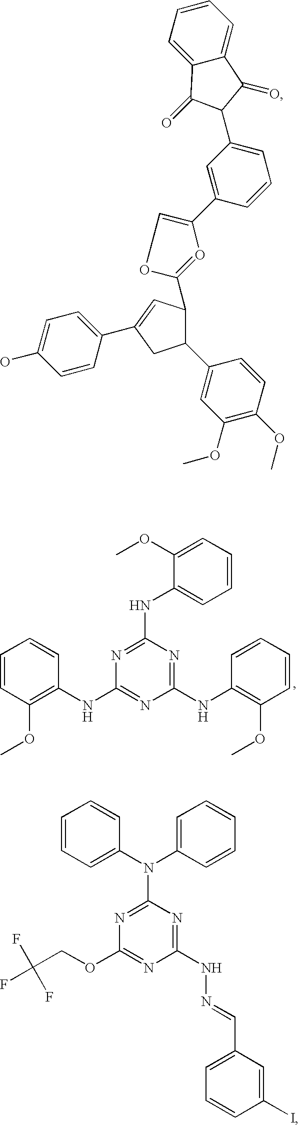

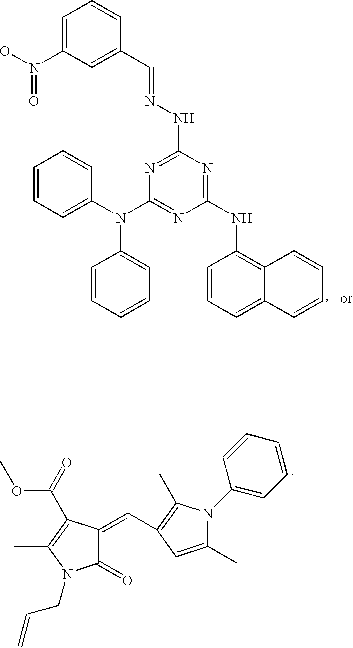

In certain embodiments, the present invention relates to the aforementioned method, wherein said compound is a compound of any one of formulae X-XXXIX as described below.

Another aspect of the present invention relates to a method of treating or preventing drug-induced phospholipidosis, comprising the step of:

administering to a patient in need thereof a therapeutically effective amount of a compound of any one of formulae I-IX, wherein formula I is represented by:

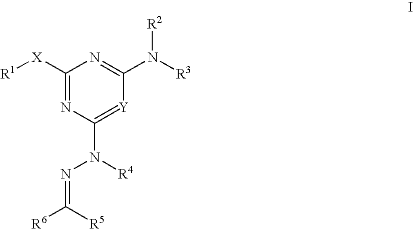

wherein,

X is O or —N(R⁷)—;

Y is N or —C(R⁸)—;

R¹ and R² represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, heteroaralkyl, —(CR⁹═CR⁹)ₙ-aryl, or —(CR⁹═CR⁹)ₙ-heteroaryl;

R⁶ is H or alkyl; or R⁵ and R⁶ taken together form a optionally substituted monocyclic or bicyclic ring having 1 or 2 heteroatoms selected from the group consisting of O, N, and S;

R⁷ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁷ taken together form a 3-8 member ring optionally substituted with one or more of alkyl, halogen, hydroxy, alkoxy, or amino;

R⁸ and R⁹ represent independently for each occurrence H or alkyl; and

n is 1 or 2;

formula II is represented by:

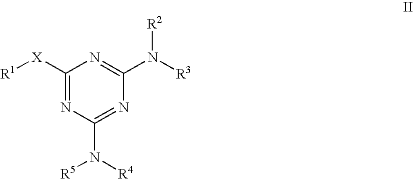

wherein,

X is O or —N(R⁶)—;

R¹ and R² represent independently cycloalkyl, heterocycloalkyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is hydrogen, alkyl, cycloalkyl, aryl, or aralkyl;

R⁵ is cycloalkyl, heterocycloalkyl, cycloalkenyl, heterocycloalkenyl, aryl, heteroaryl, aralkyl, or heteroaralkyl;

R⁶ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R⁶ taken together form a 3-8 member ring optionally substituted with one or more of alkyl, halogen, hydroxy, alkoxy, or amino;

formula III is represented by:

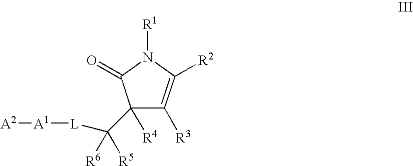

wherein,

R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);

R² is cycloalkyl, heterocycloalkyl, cycloalkenyl, heterocycloalkenyl, aryl, heteroaryl, aralkyl, heteroaralkyl, or alkyl;

R³ is hydrogen, alkyl, —CO₂R⁸, or —C(O)N(R⁷)(R⁸);

R⁴ and R⁵ represent independently H or alkyl; or R⁴ and R⁵ taken together form a bond;

R⁶ and R⁷ represent independently for each occurrence H or alkyl;

R⁸ represents independently for each occurrence alkyl, cycloalkyl, aryl, heteroaryl, aralkyl, or heteroaralkyl;

L is a bond, —C(R⁷)₂—, or —(CR⁷═CR⁷)—; and

A¹ and A² represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl; formula IV is represented by:

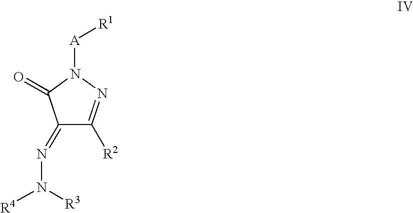

wherein,

A is cycloalkenyl, heterocycloalkenyl, aryl, heteroaryl, aralkyl, or heteroaralkyl;

R¹ is cycloalkenyl, heterocycloalkenyl, aryl, heteroaryl, aralkyl, heteroaralkyl, —(CR³═CR³)-aryl, or —(CR³═CR³)-heteroaryl;

R² is alkyl, cycloalkyl, heterocycloalkyl, cycloalkenyl, heterocycloalkenyl, aryl, heteroaryl, aralkyl, or heteroaralkyl;

R³ represents independently for each occurrence H or alkyl; and

R⁴ is cycloalkyl, heterocycloalkyl, cycloalkenyl, heterocycloalkenyl, aryl, heteroaryl, aralkyl, or heteroaralkyl; formula V is represented by:

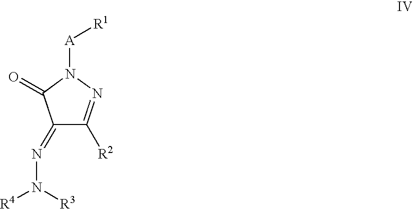

wherein,

X is O, —N(R⁵)—, —N(R⁵)C(O)—, —C(O)N(R⁵)—, —OC(O)—, —CO₂—, or —N(R⁵)CO₂—;

Y is O, S, or —N(R⁵)—;

R¹ is cycloalkenyl, heterocycloalkenyl, aryl, heteroaryl, aralkyl, or heteroaralkyl;

R² represents independently for each occurrence H or alkyl, or two R² taken together form ═O;

R³ and R⁴ represent independently cycloalkyl, heterocycloalkyl, cycloalkenyl, heterocycloalkenyl, aryl, heteroaryl, aralkyl, or heteroaralkyl;

R⁵ represents independently for each occurrence H, alkyl, aryl, or aralkyl; and

n is 1, 2, 3, 4, or 5;

formula VI is represented by:

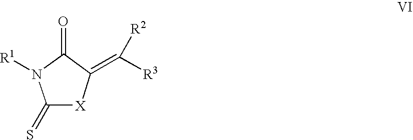

wherein,

X is O, S, or —N(R⁴)—;

R¹ is cycloalkyl, heterocycloalkyl, cycloalkenyl, heterocycloalkenyl, aryl, heteroaryl, aralkyl, heteroaralkyl, or —(C(R⁵)₂)ₙ—(CR⁵═C(R⁵)₂);

R² is cycloalkenyl, heterocycloalkenyl, aryl, heteroaryl, aralkyl, heteroaralkyl, —(CR⁵═CR⁵)-aryl, or —(CR⁵═CR⁵)-heteroaryl;

R³ is H, alkyl, alkenyl, aryl, or heteroaryl; or R² and R³ taken together form an optionally substituted monocyclic or bicyclic ring having 0, 1, or 2 heteroatoms selected from the group consisting of O, N, and S;

R⁴ and R⁵ represent independently for each occurrence H, alkyl, aryl, or aralkyl; and

n is 1, 2, 3, 4, or 5;

formula VII is represented by:

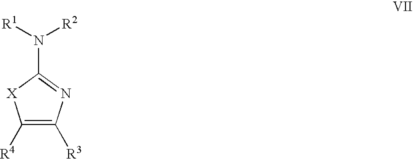

wherein,

X is O or S;

R¹ is cycloalkenyl, heterocycloalkenyl, aryl, heteroaryl, aralkyl, heteroaralkyl, or —C(O)R⁵;

R² is H or alkyl;

R³ is cycloalkenyl, heterocycloalkenyl, aryl, heteroaryl, aralkyl, heteroaralkyl, or an optionally substituted bicyclic ring having 1 or 2 heteroatoms selected form the group consisting of O, N, and S;

R⁴ is H, alkyl, —CO₂R⁶, or —C(O)N(R⁶)₂;

R⁵ is cycloalkenyl, heterocycloalkenyl, heteroaryl, aralkyl, or heteroaralkyl; or R⁵ is an aryl group optionally substituted with one or more of alkyl, halogen, —OR⁶, —N(R⁶)₂, —CO₂R⁶, C(O)N(R⁶)₂, cyano, or nitro; and

R⁶ represents independently for each occurrence H, alkyl, cycloalkyl, heterocycloalkyl, aryl, heteroaryl, aralkyl, or heteroaralkyl;

formula VIII is represented by:

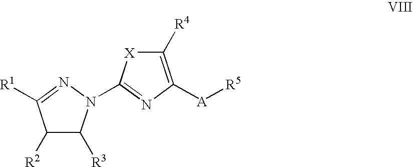

wherein,

X is O or S;

R¹, R³, and A represent independently cycloalkenyl, heterocycloalkenyl, aryl, heteroaryl, aralkyl, or heteroaralkyl;

R² and R⁴ represent independently H or alkyl;

R⁵ is an optionally substituted monocyclic or bicyclic ring having 1, 2, or 3 heteroatoms selected from the group consisting of O, N, and S; and

formula IX is represented by:

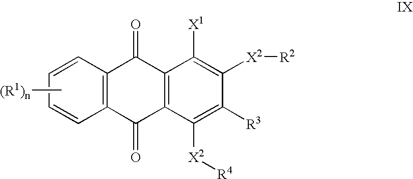

wherein,

X¹ is —OR⁵, —SR⁵, or —N(R⁵)₂;

X² represents independently for each occurrence O, S, or —N(R⁵)—;

R¹ represents independently for each occurrence alkyl, halogen, nitro, cyano, alkoxyl, alkenyl, alkynyl, cycloalkenyl, heterocycloalkenyl, aryl, heteroaryl, aralkyl, or heteroaralkyl, —N(R⁵)₂, —OH, —C(O)R⁶, —CO₂R⁵, or C(O)N(R⁵)₂;

R² and R⁴ represent independently cycloalkenyl, heterocycloalkenyl, aryl, heteroaryl, aralkyl, or heteroaralkyl;

R³ is H, alkyl, or halogen;

R⁵ represents independently for each occurrence H, alkyl, aryl, heteroaryl, aralkyl, or heteroaralkyl;

R⁶ represents independently for each occurrence alkyl, cycloalkyl, heterocycloalkyl, aryl, heteroaryl, aralkyl, or heteroaralkyl; and

n is 0, 1, 2, 3, or 4.

In certain embodiments, the present invention relates to the aforementioned method, wherein said compound is a compound of formula I.

In certain embodiments, the present invention relates to the aforementioned method, wherein said compound is a compound of formula I, X is O or —N(R⁷)—; Y is N; R¹ and R² represent independently alkyl, haloalkyl, or aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is —N(R⁷)—; Y is N; R¹ and R² are aryl; R³ is aryl; or R² and R³ taken together form a 3-8 member ring optionally substituted with one or more of alkyl, halogen, hydroxy, alkoxy, or amino; R⁴ is hydrogen; R⁵ is heterocycloalkyl or aryl; R⁶ is H or alkyl; or R⁵ and R⁶ taken together form a optionally substituted monocyclic or bicyclic ring having 1 or 2 heteroatoms selected from the group consisting of O, N, and S; and R⁷ is hydrogen; or R¹ and R⁷ taken together form a 3-8 member ring optionally substituted with one or more of alkyl, halogen, hydroxy, alkoxy, or amino.

In certain embodiments, the present invention relates to the aforementioned method, wherein said compound is a compound of formula I, X is O or —N(R⁷)—; Y is —C(R⁸)—; R¹ and R² represent independently alkyl, heteroalkyl, or haloalkyl; R³ is hydrogen, alkyl, heteroalkyl, or haloalkyl; R⁴ is hydrogen; R⁵ is heteroaryl; R⁶ is H or alkyl; R⁷ is hydrogen, alkyl, heteroalkyl, or haloalkyl; and R⁸ is H or alkyl.

In certain embodiments, the present invention relates to the aforementioned method, wherein said compound is a compound of formula II.

In certain embodiments, the present invention relates to the aforementioned method, wherein said compound is a compound of formula II, X is —N(R⁶)—; R¹, R² and R⁵ represent independently aryl or heteroaryl; and R³, R⁴, and R⁶ represent independently hydrogen or alkyl.

In certain embodiments, the present invention relates to the aforementioned method, wherein said compound is a compound of formula III.

In certain embodiments, the present invention relates to the aforementioned method, wherein said compound is a compound of formula III; R¹, R², A¹, and A² represent independently aryl or heteroaryl; R³ is hydrogen or alkyl; R⁶ is H or alkyl; and L is a bond.

In certain embodiments, the present invention relates to the aforementioned method, wherein said compound is a compound of formula III, R¹ is —(C(R⁷)₂)ₙ—(CR⁷═C(R⁷)₂); R² is alkyl; R³ is alkyl, —CO₂R⁸, or —C(O)N(R⁷)(R⁸); R⁴ and R⁵ represent independently H or alkyl; or R⁴ and R⁵ taken together form a bond; R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

In certain embodiments, the present invention relates to the aforementioned method, wherein said compound is a compound of formula III, R¹ comprises a carboxylic acid group; R¹ is a carboxylic acid substituted aryl; R¹ is a carboxylic acid substituted phenyl; and/or R¹ is a para-substituted carboxylic acid phenyl.

In certain embodiments, the present invention relates to the aforementioned method, wherein said compound is a compound of formula IV.

In certain embodiments, the present invention relates to the aforementioned method, wherein said compound is a compound of formula IV; A, R¹, and R⁴ represent independently aryl or heteroaryl; R¹ is cycloalkenyl, heterocycloalkenyl, aryl, heteroaryl, aralkyl, heteroaralkyl, —(CR³═CR³)-aryl, or —(CR³═CR³)-heteroaryl; R² is alkyl or aryl; and R³ represents independently for each occurrence H or alkyl.

In certain embodiments, the present invention relates to the aforementioned method, wherein said compound is a compound of formula V.

In certain embodiments, the present invention relates to the aforementioned method, wherein said compound is a compound of formula V; X is —C(O)N(R⁵)— or —CO₂—; Y is O or S; R¹, R³, and R⁴ represent independently aryl or heteroaryl; R² represents independently for each occurrence H or alkyl, or two R² taken together form ═O; R³ and R⁴ represent independently cycloalkyl, heterocycloalkyl, cycloalkenyl, heterocycloalkenyl, aryl, heteroaryl, aralkyl, or heteroaralkyl; R⁵ represents independently for each occurrence H, alkyl, aryl, or aralkyl; and n is 1 or 2;

In certain embodiments, the present invention relates to the aforementioned method, wherein said compound is a compound of formula VI.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is aryl, heteroaryl, —(CR⁵═CR⁵)-aryl or —(CR⁵═CR⁵)-heteroaryl; R³ is H or alkyl; R⁵ represents independently for each occurrence H, alkyl, aryl, or aralkyl; and n is 1 or 2.

In certain embodiments, the present invention relates to the aforementioned method, wherein said compound is a compound of formula VI, X is S, R¹ is aryl, R² is aryl, and R³ is H or alkyl.

In certain embodiments, the present invention relates to the aforementioned method, wherein said compound is a compound of formula VI, X is S, R¹ is alkoxy-substituted phenyl, R² is dialkylamino-substituted phenyl, and R³ is H.

In certain embodiments, the present invention relates to the aforementioned method, wherein said compound is a compound of formula VI; X is S; R¹ is aryl, heteroaryl, or —(C(R⁵)₂)ₙ—(CR⁵═C(R⁵)₂); R² is —(CR⁵═CR⁵)-aryl or —(CR⁵═CR⁵)-heteroaryl; R³ is H or alkyl; or R² and R³ taken together form an optionally substituted monocyclic or bicyclic ring having 0, 1, or 2 heteroatoms selected from the group consisting of O, N, and S; R⁵ represents independently for each occurrence H, alkyl, aryl, or aralkyl; and n is 1 or 2.

In certain embodiments, the present invention relates to the aforementioned method, wherein said compound is a compound of formula VII.

In certain embodiments, the present invention relates to the aforementioned method, wherein said compound is a compound of formula VII; X is O or S; R¹ is aryl, heteroaryl, or —C(O)R⁵; R² is H or alkyl; R³ is aryl, heteroaryl, or an optionally substituted bicyclic ring having 1 or 2 heteroatoms selected form the group consisting of O, N, and S; R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; and R⁶ represents independently for each occurrence H, alkyl, aryl, heteroaryl, aralkyl, or heteroaralkyl.

In certain embodiments, the present invention relates to the aforementioned method, wherein said compound is a compound of formula VII; X is O or S; R¹ is aryl, heteroaryl, or —C(O)R⁵; R² is H or alkyl; R³ is represented by

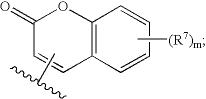

R⁴ is H, alkyl, —CO₂R⁶, or —C(O)N(R⁶)₂; R⁵ is an aryl group optionally substituted with one or more of alkyl, halogen, —OR⁶, —N(R⁶)₂, —CO₂R⁶, C(O)N(R⁶)₂, cyano, or nitro; R⁶ represents independently for each occurrence H, alkyl, aryl, heteroaryl, aralkyl, or heteroaralkyl; m is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula VIII.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; and R⁵ is an optionally substituted bicyclic ring having 1, 2, or 3 heteroatoms selected from the group consisting of O, N, and S.

In certain embodiments, the present invention relates to the aforementioned method, wherein said compound is a compound of formula VIII; X is O or S; R¹, R³, and A represent independently aryl or heteroaryl; R² and R⁴ represent independently H or alkyl; R⁵ is represented by

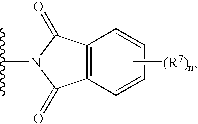

wherein n is 0, 1, 2, 3, or 4; and R⁷ represents independently for each occurrence halogen, hydroxyl, amino, carboxyl, nitro, cyano, alkyl, or alkoxyl.

In certain embodiments, the present invention relates to the aforementioned method, wherein said compound is a compound of formula IX;

In certain embodiments, the present invention relates to the aforementioned method, wherein said compound is a compound of formula IX; X¹ is —N(R⁵)₂; X² represents independently for each occurrence O, or S; R¹ represents independently for each occurrence alkyl, halogen, nitro, cyano, alkoxyl, —N(R⁵)₂, —OH, —C(O)R⁶, —CO₂R⁵, or C(O)N(R⁵)₂; R² and R⁴ represent independently aryl, heteroaryl, aralkyl, or heteroaralkyl; R³ is H, alkyl, or halogen; R⁵ represents independently for each occurrence H, alkyl, aryl, heteroaryl, aralkyl, or heteroaralkyl; R⁶ represents independently for each occurrence alkyl, cycloalkyl, heterocycloalkyl, aryl, heteroaryl, aralkyl, or heteroaralkyl; and n is 0, 1, 2, 3, or 4.

In certain embodiments, the present invention relates to the aforementioned method, wherein said compound is

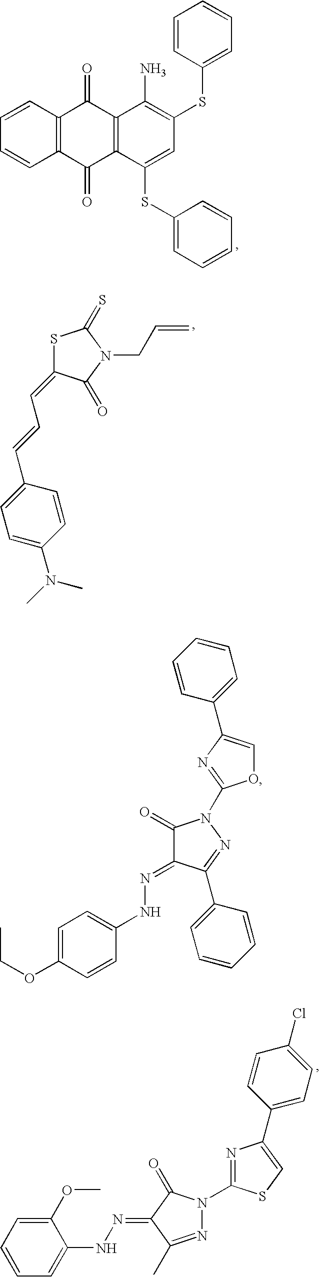

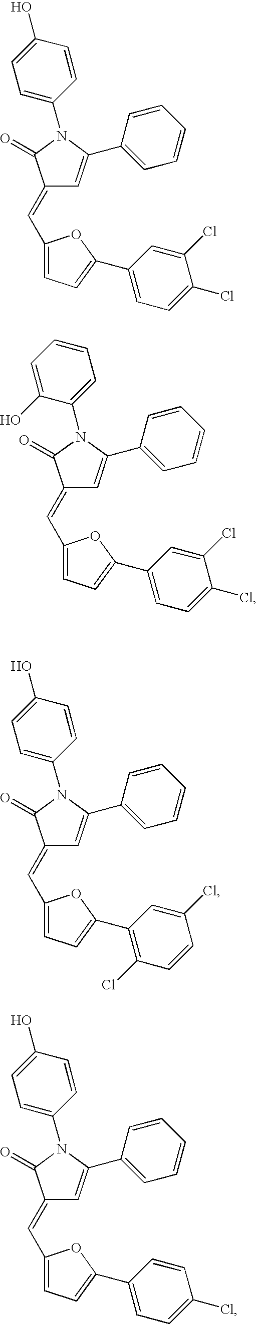

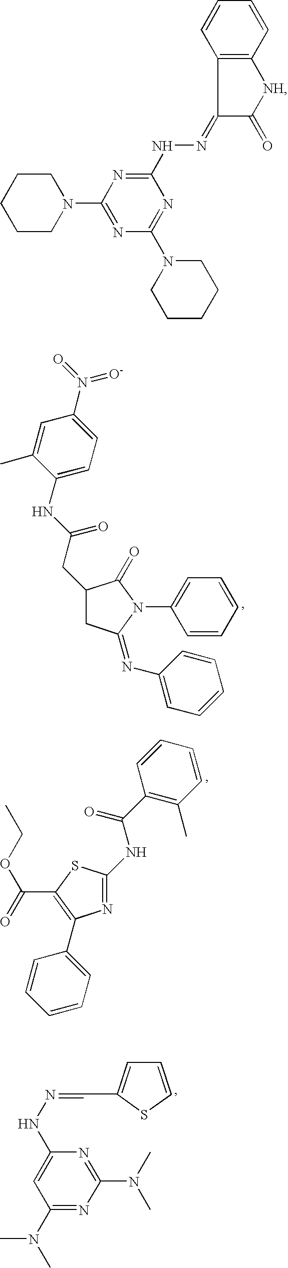

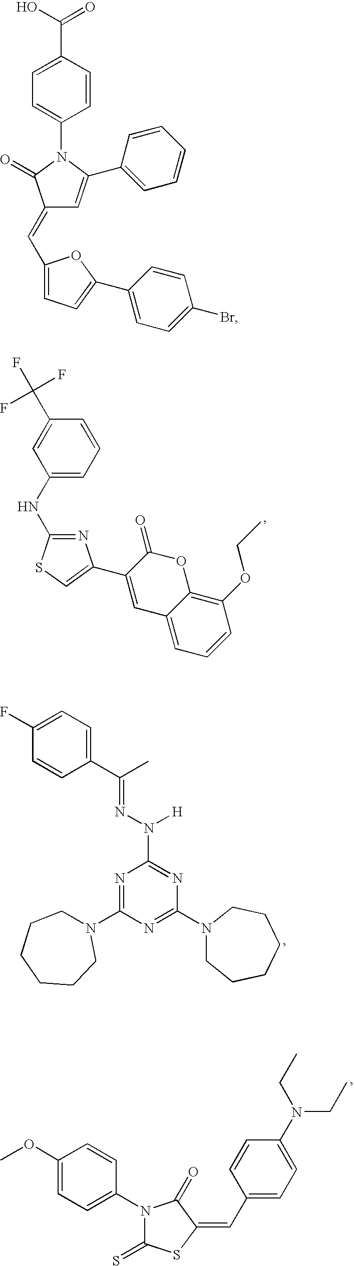

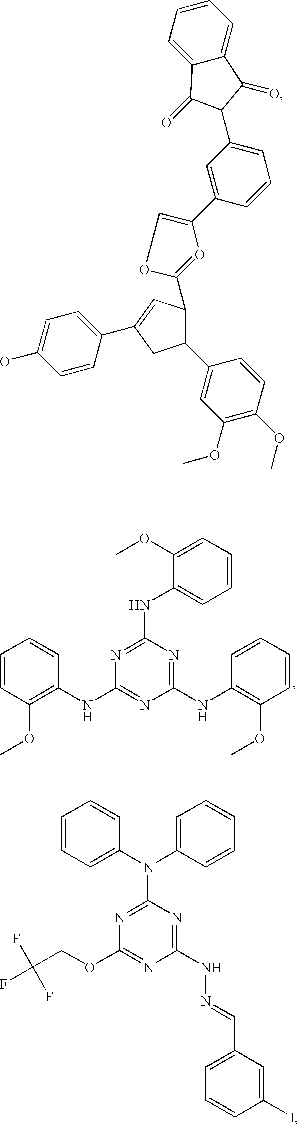

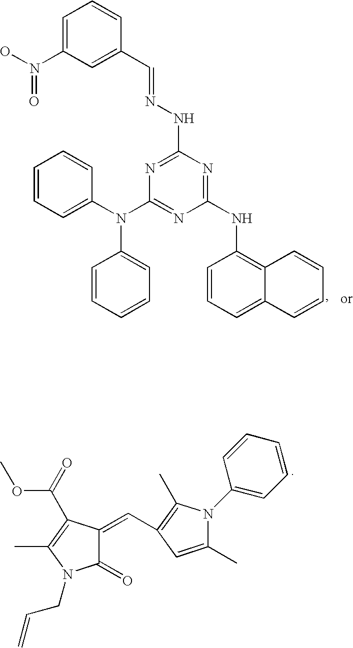

In certain embodiments, the present invention relates to the aforementioned method, wherein said compound is a compound of any one of formulae X-XXXIX as described below.

In certain embodiments, the present invention relates to the aforementioned method, wherein said compound is

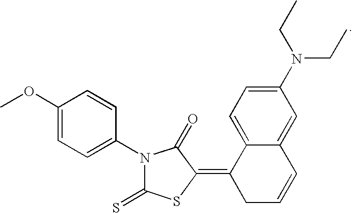

In certain embodiments, the present invention relates to any one of the aforementioned methods, wherein said patient is a mammal.

In certain embodiments, the present invention relates to any one of the aforementioned methods, wherein said patient is a primate, equine, canine, or feline.

In certain embodiments, the present invention relates to any one of the aforementioned methods, wherein said patient is a human.

Compounds & Compositions of the Invention

One aspect of the present invention relates to a compound represented by formula X:

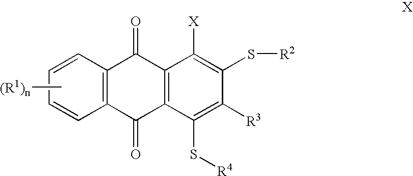

wherein,

X is OH or N(R⁵)₂;

R¹ represents independently for each occurrence alkyl, halogen, nitro, cyano, alkoxyl, alkenyl, alkynyl, cycloalkenyl, heterocycloalkenyl, aryl, heteroaryl, aralkyl, or heteroaralkyl, —N(R⁵)₂, —OH, —C(O)R⁶, —CO₂R⁵, or C(O)N(R⁵)₂;

R² and R⁴ represent independently cycloalkenyl, heterocycloalkenyl, aryl, aralkyl, heteroaralkyl, or heteroaryl having 1 heteroatom selected form the group consisting of N, O, or S;

R³ is H, alkyl, or halogen;

R⁵ represents independently for each occurrence H, alkyl, aryl, heteroaryl, aralkyl, or heteroaralkyl;

R⁶ represents independently for each occurrence alkyl, cycloalkyl, heterocycloalkyl, aryl, heteroaryl, aralkyl, or heteroaralkyl;

n is 0, 1, 2, 3, or 4; and

provided that when X is NH₂, at least of one of R² and R⁴ is not aryl.

In certain embodiments, the present invention relates to the aforementioned compound, wherein, wherein X is NH₂ and R² is aryl.

In certain embodiments, the present invention relates to the aforementioned compound, wherein X is NH₂ and R⁴ is aryl.

Another aspect of the invention relates to a compound represented by formula XI:

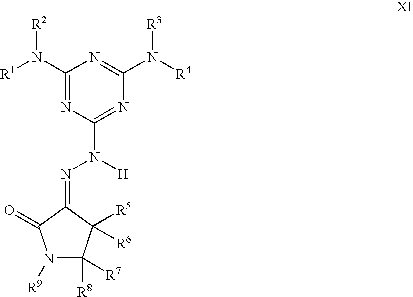

wherein,

R¹ and R³ represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R² and R⁴ represent independently hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R² taken together form a 3-8 member ring; or R³ and R⁴ taken together form a 3-8 member ring;

R⁵, R⁶, R⁷, and R⁸ represent independently hydrogen, alkyl, cycloalkyl, aryl, heteroaryl, aralkyl, or heteroaralkyl; or R⁵, R⁶, R⁷, and R⁸ taken together form an aryl or heteroaryl group substituted with at least one functional group selected from the group consisting of (C₂-C₆)alkyl, halogen, nitro, cyano, alkoxyl, alkenyl, alkynyl, cycloalkenyl, heterocycloalkenyl, aryl, heteroaryl, aralkyl, or heteroaralkyl, —N(R⁹)₂, —OR⁹, —C(O)R⁹, —CO₂R⁹, or C(O)N(R⁹)₂; and

R⁹ represents independently for each occurrence H, alkyl, aryl, heteroaryl, aralkyl, or heteroaralkyl.

In certain embodiments, the present invention relates to the aforementioned compound, wherein R¹ and R² form a 6 membered ring.

In certain embodiments, the present invention relates to the aforementioned compound, wherein R³ and R⁴ form a 6 membered ring.

In certain embodiments, the present invention relates to the aforementioned compound, wherein R⁵, R⁶, R⁷, and R⁸ taken together form an aryl ring.

Another aspect of the invention relates to a compound represented by formula XII:

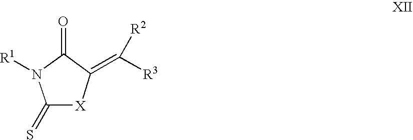

wherein,

X is O, S, or —N(R⁴)—;

R¹ is cycloalkyl, heterocycloalkyl, cycloalkenyl, heterocycloalkenyl, heteroaryl, aralkyl, heteroaralkyl;

R² is cycloalkenyl, heterocycloalkenyl, aryl, heteroaryl, aralkyl, heteroaralkyl, —(CR⁵═CR⁵)-aryl, or —(CR⁵═CR⁵)-heteroaryl;

R³ is H, alkyl, alkenyl, aryl, or heteroaryl; or R² and R³ taken together form an optionally substituted monocyclic or bicyclic ring having 0, 1, or 2 heteroatoms selected from the group consisting of O, N, and S;

R⁴ and R⁵ represent independently for each occurrence H, alkyl, aryl, or aralkyl; and

n is 1, 2, 3, 4, or 5;

or a compound represented by formula XIII:

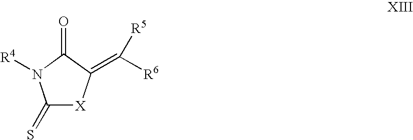

wherein,

X is O, S, or —N(R⁷)—;

R⁴ is —(C(R⁸)₂)ₙ—(CR⁸═C(R⁸)₂);

R⁵ is cycloalkenyl, heterocycloalkenyl, aryl, heteroaryl, aralkyl, heteroaralkyl, or —(CR⁸═C(R⁸)₂);

R⁶ is H, alkyl, alkenyl, aryl, or heteroaryl; or R⁵ and R⁶ taken together form an optionally substituted monocyclic or bicyclic ring having 0, 1, or 2 heteroatoms selected from the group consisting of O, N, and S;

R⁷ and R⁸ represent independently for each occurrence H, alkyl, cycloalkyl, or heterocycloalkyl, aralkyl, or heteroaralkyl; and

n is 1, 2, 3, 4, or 5;

or a compound of formula XIV:

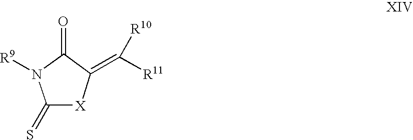

wherein,

X is O, S, or —N(R¹²)—;

R⁹ is cycloalkyl, heterocycloalkyl, cycloalkenyl, heterocycloalkenyl, -aryl-OR¹⁴, heteroaryl, aralkyl, or heteroaralkyl;

R¹⁰ is cycloalkenyl, heterocycloalkenyl, aryl, heteroaryl, aralkyl, heteroaralkyl, —(CR¹³═CR¹³)-aryl, or —(CR¹³═CR¹³)-heteroaryl;

R¹¹ is H, alkyl, alkenyl, aryl, or heteroaryl;

R¹² and R¹³ represent independently for each occurrence H, alkyl, aryl, or aralkyl;

R¹⁴ is heteroalkyl, heterocycloalkyl, heterocycloalkenyl, heteroaryl, aralkyl, or heteroaralkyl; and

n is 1, 2, 3, 4, or 5.

In certain embodiments, the present invention relates to the aforementioned compound having formula XIII, wherein X is S and R⁴ is allyl.

In certain embodiments, the present invention relates to the aforementioned compound having formula XIII, wherein X is S, R⁴ is allyl and R⁵ is —(CR⁸═C(R⁸)₂).

In certain embodiments, the present invention relates to the aforementioned compound having formula XIV, wherein X is S and R⁹ is -aryl-OR⁴.

In certain embodiments, the present invention relates to the aforementioned compound having formula XIV, wherein X is S, R⁹ is -aryl-OR¹⁴, R¹⁰ is aryl, and R¹¹ is H. Another aspect of the invention relates to a compound represented by formula XIVa:

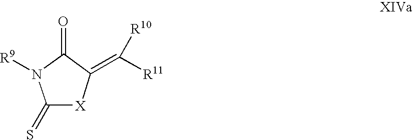

wherein,

X is O, S, or —N(R¹²)—;

R⁹ is cycloalkyl, heterocycloalkyl, cycloalkenyl, heterocycloalkenyl, aryl, heteroaryl, aralkyl, or heteroaralkyl;

R¹⁰ is cycloalkenyl, heterocycloalkenyl, aryl, heteroaryl, aralkyl, heteroaralkyl, —(CR¹³═CR¹³)-aryl, or —(CR¹³═CR¹³)-heteroaryl;

R¹¹ is H, alkyl, alkenyl, aryl, or heteroaryl; or R¹⁰ and R¹¹ taken together form an optionally substituted monocyclic or bicyclic ring having 0, 1, or 2 heteroatoms selected from the group consisting of O, N, and S;

R¹² and R¹³ represent independently for each occurrence H, alkyl, aryl, or aralkyl; and

n is 1, 2, 3, 4, or 5.

In certain embodiments, the present invention relates to the aforementioned compound having formula XIVa, wherein X is S and R⁹ is aryl.

Another aspect of the invention relates to a compound represented by formula XV:

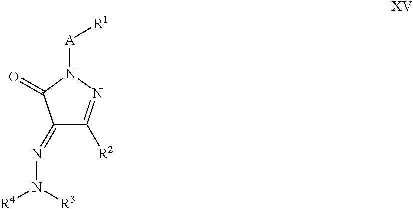

wherein,

A is cycloalkenyl, heterocycloalkenyl, aryl, heteroaryl, aralkyl, or heteroaralkyl;

R¹ is cycloalkenyl, heterocycloalkenyl, aryl, heteroaryl, aralkyl, heteroaralkyl, —(CR³═CR³)-aryl, or —(CR³═CR³)-heteroaryl;

R² is cycloalkyl, heterocycloalkyl, cycloalkenyl, heterocycloalkenyl, heteroaryl, aralkyl, or heteroaralkyl;

R³ represents independently for each occurrence H or alkyl; and

R⁴ is cycloalkyl, heterocycloalkyl, cycloalkenyl, heterocycloalkenyl, aryl, heteroaryl, aralkyl, or heteroaralkyl;

or a compound represented by formula XVI:

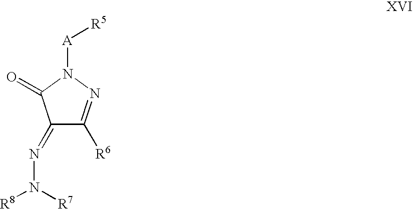

wherein,

A is cycloalkenyl, heterocycloalkenyl, aryl, heteroaryl, aralkyl, or heteroaralkyl;

R⁵ is cycloalkenyl, heterocycloalkenyl, heteroaryl, aralkyl, heteroaralkyl, —(CR⁷═CR⁷)-aryl, or —(CR⁷═CR⁷)-heteroaryl;

R⁶ is alkyl or aryl;

R⁷ represents independently for each occurrence H or alkyl; and

R⁸ is cycloalkyl, heterocycloalkyl, cycloalkenyl, heterocycloalkenyl, aryl, heteroaryl, aralkyl, or heteroaralkyl; or a compound represented by formula XVII:

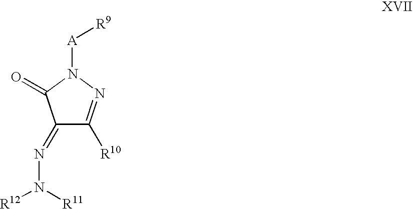

wherein,

A is cycloalkenyl, heterocycloalkenyl, aryl, heteroaryl, aralkyl, or heteroaralkyl;

R⁹ is cycloalkenyl, heterocycloalkenyl, aryl, heteroaryl, aralkyl, heteroaralkyl, —(CR¹¹═CR¹¹)-aryl, or —(CR¹¹═CR¹¹)-heteroaryl;

R¹⁰ is alkyl or aryl;

R¹¹ represents independently for each occurrence H or alkyl; and

R¹² is cycloalkyl, heterocycloalkyl, cycloalkenyl, heterocycloalkenyl, heteroaryl, aralkyl, or heteroaralkyl.

In certain embodiments, the present invention relates to the aforementioned compound having formula XVI, wherein A is heteroaryl and R⁶ is aryl.

In certain embodiments, the present invention relates to the aforementioned compound having formula XVI, wherein A is heteroaryl and R⁶ is alkyl.

In certain embodiments, the present invention relates to the aforementioned compound having formula XVII, wherein A is heteroaryl, R⁹ is aryl, and R¹⁰ is alkyl.

In certain embodiments, the present invention relates to the aforementioned compound having formula XVII, wherein A is heteroaryl, R⁹ is aryl, and R¹⁰ is aryl.

Another aspect of the invention relates to a compound represented by formula XVIII:

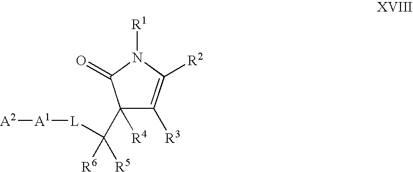

wherein,

R¹ is cycloalkyl, heterocycloalkyl, cycloalkenyl, heterocycloalkenyl, aryl, heteroaryl, aralkyl, heteroaralkyl;

R² is cycloalkyl, heterocycloalkyl, cycloalkenyl, heterocycloalkenyl, heteroaryl, aralkyl, heteroaralkyl, or alkyl;

R³ is hydrogen, alkyl, —CO₂R⁸, or —C(O)N(R⁷)(R⁸);

R⁴ and R⁵ represent independently H or alkyl; or R⁴ and R⁵ taken together form a bond;

R⁶ and R⁷ represent independently for each occurrence H or alkyl;

R⁸ represents independently for each occurrence alkyl, cycloalkyl, aryl, heteroaryl, aralkyl, or heteroaralkyl;

L is a bond, —C(R⁷)₂—, or —(CR⁷═CR⁷)—; and A¹ and A² represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;

or a compound represented by formula XIX:

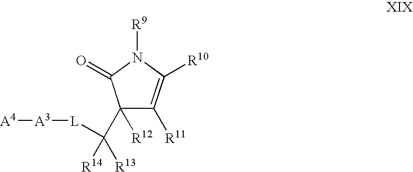

wherein,

R⁹ is cycloalkyl, heterocycloalkyl, cycloalkenyl, heterocycloalkenyl, heteroaryl, aralkyl, heteroaralkyl, or —(C(R¹⁵)₂)ₙ—(CR¹⁵═C(R¹⁵)₂);

R¹⁰ is aryl;

R¹¹ is hydrogen, alkyl, —CO₂R¹⁶, or —C(O)N(R¹⁵)(R¹⁶);

R¹² and R¹³ represent independently H or alkyl; or R¹² and R¹³ taken together form a bond;

R¹⁴ and R¹⁵ represent independently for each occurrence H or alkyl;

R¹⁶ represents independently for each occurrence alkyl, cycloalkyl, aryl, heteroaryl, aralkyl, or heteroaralkyl;

L is a bond, —C(R¹⁵)₂—, or —(CR¹⁵═CR¹⁵)—;

A³ represents a bivalent cycloalkenyl, heterocycloalkenyl, aryl, heteroaryl, aralkyl, heteroaralkyl, —(CR¹⁵═CR¹⁵)-aryl-, or —(CR¹⁵═CR¹⁵)-heteroaryl-; and

A⁴ represents cycloalkenyl, heterocycloalkenyl, aryl, heteroaryl, aralkyl, heteroaralkyl, —(CR¹⁵═CR¹⁵)-aryl, or —(CR¹⁵═CR¹⁵)-heteroaryl; or a compound represented by formula XX:

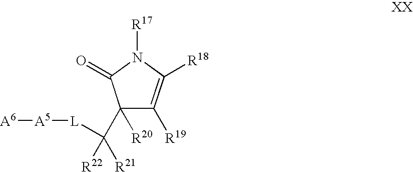

wherein,

R¹⁷ is cycloalkyl, heterocycloalkyl, cycloalkenyl, heterocycloalkenyl, aryl, heteroaryl, aralkyl, heteroaralkyl, or —(C(R²³)₂)ₙ—(CR²³═C(R²³)₂);

R¹⁸ is aryl;

R¹⁹ is hydrogen, alkyl, —CO₂R²⁴, or —C(O)N(R²³)(R²⁴);

R²⁰ and R²¹ represent independently H or alkyl; or R²⁰ and R²¹ taken together form a bond;

R²² and R²³ represent independently for each occurrence H or alkyl;

R²⁴ represents independently for each occurrence alkyl, cycloalkyl, aryl, heteroaryl, aralkyl, or heteroaralkyl;

L is a bond, —C(R²³)₂—, or —(CR²³═CR²³)—;

A⁵ represents a bivalent cycloalkenyl, heterocycloalkenyl, aryl, heteroaryl, aralkyl, heteroaralkyl, —(CR²³═CR²³)-aryl-, or —(CR²³═CR²³)-heteroaryl-; and

A⁶ represents cycloalkenyl, heterocycloalkenyl, heteroaryl, aralkyl, heteroaralkyl, —(CR²³═CR²³)-aryl, or —(CR²³═CR²³)-heteroaryl; or a compound of formula XXI:

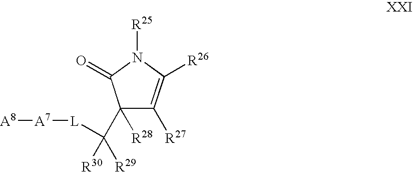

wherein,

R²⁵ is —(C(R³¹)₂)ₙ—(CR³¹═C(R³¹)₂);

R²⁶ is cycloalkyl, heterocycloalkyl, cycloalkenyl, heterocycloalkenyl, heteroaryl, aralkyl, or heteroaralkyl;

R²⁷ is hydrogen, alkyl, —CO₂R³², or —C(O)N(R³¹)(R³²);

R²⁸ and R²⁹ represent independently H or alkyl; or R²⁸ and R²⁹ taken together form a bond;

R³⁰ and R³¹ represent independently for each occurrence H or alkyl;

R³² represents independently for each occurrence alkyl, cycloalkyl, aryl, heteroaryl, aralkyl, or heteroaralkyl;

L is a bond, —C(R³¹)₂—, or —(CR³¹═CR³¹)—; and

A⁷ and A⁸ represent independently cycloalkenyl, heterocycloalkenyl, aryl, heteroaryl, aralkyl, heteroaralkyl, —(CR³¹═CR³¹)-aryl, or —(CR³¹═CR³¹)-heteroaryl.

In certain embodiments, the present invention relates to the aforementioned compound having formula XVIII, wherein R¹ is aryl.

In certain embodiments, the present invention relates to the aforementioned compound having formula XVIII, wherein R¹ is aryl, and R⁴ and R⁵ taken together form a bond.

In certain embodiments, the present invention relates to the aforementioned compound having formula XVIII, wherein R¹ is aryl, R⁴ and R⁵ taken together form a bond, L is a bond, and A¹ is heteroaryl.

In certain embodiments, the present invention relates to the aforementioned compound having formula XVIII, wherein R¹ is aryl, R⁴ and R⁵ taken together form a bond, L is a bond, A¹ is heteroaryl, and A² is aryl.

In certain embodiments, the present invention relates to the aforementioned compound having formula XVIII, wherein R¹ comprises a carboxylic acid group; R¹ is a carboxylic acid substituted aryl; R¹ is a carboxylic acid substituted phenyl; and/or R¹ is a para-substituted carboxylic acid phenyl.

In certain embodiments, the present invention relates to the aforementioned compound having formula XX, wherein R¹⁷ comprises a carboxylic acid group; R¹⁷ is a carboxylic acid substituted aryl; R¹⁷ is a carboxylic acid substituted phenyl; and/or R¹⁷ is a para-substituted carboxylic acid phenyl.

In certain embodiments, the present invention relates to the aforementioned compound having formula XXI, wherein R²⁵ is allyl.

In certain embodiments, the present invention relates to the aforementioned compound having formula XXI, wherein R²⁵ is allyl and R²⁷ is —CO₂R³².

In certain embodiments, the present invention relates to the aforementioned compound having formula XXI, wherein R²⁵ is allyl, R²⁷ is —CO₂R³², and A⁷ is heteroaryl.

In certain embodiments, the present invention relates to the aforementioned compound having formula XXI, wherein R²⁵ is allyl, R²⁷ is —CO₂R³², A⁷ is heteroaryl, and A⁸ is aryl.

Another aspect of the invention relates to a compound represented by formula XXII:

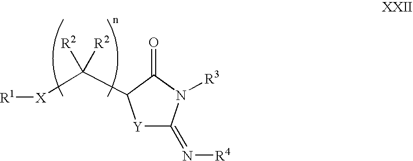

wherein,

X is O, —N(R⁵)—, —N(R⁵)C(O)—, —C(O)N(R⁵)—, —OC(O)—, —CO₂—, or —N(R⁵)CO₂—;

Y is O, S, or —N(R⁵)—;

R¹ is cycloalkenyl, heterocycloalkenyl, aryl, heteroaryl, aralkyl, or heteroaralkyl;

R² represents independently for each occurrence H or alkyl, or two R² taken together form ═O;

R³ represents cycloalkyl, heterocycloalkyl, cycloalkenyl, heterocycloalkenyl, heteroaryl, aralkyl, or heteroaralkyl;

R⁴ represents cycloalkyl, heterocycloalkyl, cycloalkenyl, heterocycloalkenyl, aryl, heteroaryl, aralkyl, or heteroaralkyl

R⁵ represents independently for each occurrence H, alkyl, aryl, or aralkyl; and

n is 1, 2, 3, 4, or 5;

or a compound of formula XXIII:

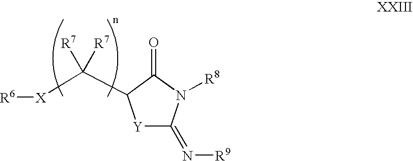

wherein,

X is O, —N(R¹⁰)—, —N(R¹⁰)C(O)—, —C(O)N(R¹⁰)—, —OC(O)—, —CO₂—, or —N(R¹⁰)CO₂—;

Y is O, S, or —N(R¹⁰)—;

R⁶ is cycloalkenyl, heterocycloalkenyl, aryl, heteroaryl, aralkyl, or heteroaralkyl;

R⁷ represents independently for each occurrence H or alkyl, or two R⁷ taken together form ═O;

R⁸ represents aryl;

R⁹ represents cycloalkyl, heterocycloalkyl, cycloalkenyl, heterocycloalkenyl, heteroaryl, aralkyl, or heteroaralkyl;

R¹⁰ represents independently for each occurrence H, alkyl, aryl, or aralkyl; and

n is 1, 2, 3, 4, or 5;

or a compound of formula XXIV:

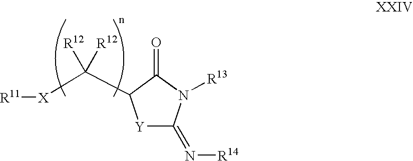

wherein,

X is O, —N(R¹⁵)—, —N(R¹⁵)C(O)—, —C(O)N(R¹⁵)—, —OC(O)—, —CO₂—, or —N(R¹⁵)CO₂—;

Y is O, S, or —N(R¹⁵)—;

R¹¹ is cycloalkenyl, heterocycloalkenyl, heteroaryl, aralkyl, or heteroaralkyl;

R¹² represents independently for each occurrence H or alkyl, or two R¹² taken together form ═O;

R¹³ represents aryl;

R¹⁴ represents cycloalkyl, heterocycloalkyl, cycloalkenyl, heterocycloalkenyl, heteroaryl, aryl, aralkyl, or heteroaralkyl;

R¹⁵ represents independently for each occurrence H, alkyl, aryl, or aralkyl; and

n is 1, 2, 3, 4, or 5.

In certain embodiments, the present invention relates to the aforementioned compound having formula XXII, wherein R⁴ is aryl, X is NH, and R¹ is aryl.

In certain embodiments, the present invention relates to the aforementioned compound having formula XXIII, wherein R⁹ is aryl, X is NH, and R⁶ is aryl.

In certain embodiments, the present invention relates to the aforementioned compound having formula XXIV, wherein R¹³ is aryl, R¹⁴ is aryl, and X is NH.

Another aspect of the invention relates to a compound represented by formula XXV:

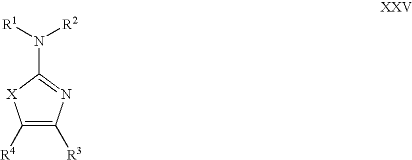

wherein,

X is O or S;

R¹ is cycloalkenyl, heterocycloalkenyl, heteroaryl, aralkyl, heteroaralkyl, or —C(O)R⁵;

R² is H or alkyl;

R³ is cycloalkenyl, heterocycloalkenyl, heteroaryl, aralkyl, heteroaralkyl, or an optionally substituted bicyclic ring having 1 or 2 heteroatoms selected form the group consisting of O, N, and S;

R⁴ is H, alkyl, —CO₂R⁶, or —C(O)N(R⁶)₂;

R⁵ is cycloalkenyl, heterocycloalkenyl, heteroaryl, aralkyl, or heteroaralkyl; or R⁵ is an aryl group optionally substituted with one or more of alkyl, halogen, —OR⁶, —N(R⁶)₂, —CO₂R⁶, C(O)N(R⁶)₂, cyano, or nitro; and

R⁶ represents independently for each occurrence H, alkyl, cycloalkyl, heterocycloalkyl, aryl, heteroaryl, aralkyl, or heteroaralkyl; or a compound of formula XXVI:

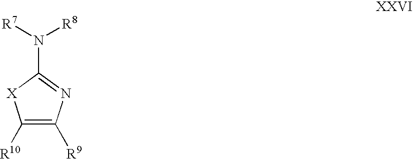

wherein,

X is O or S;

R⁷ is cycloalkenyl, heterocycloalkenyl, aryl, heteroaryl, aralkyl, heteroaralkyl, or —C(O)R¹¹;

R⁸ is H or alkyl;

R⁹ is aryl;

R¹⁰ is H, alkyl, or —C(O)N(R¹²)₂;

R¹¹ is cycloalkenyl, heterocycloalkenyl, heteroaryl, aralkyl, or heteroaralkyl; or R¹¹ is an aryl group optionally substituted with one or more of alkyl, halogen, —OR¹², —N(R²)₂, —CO₂R¹², C(O)N(R¹²)₂, cyano, or nitro; and

R¹² represents independently for each occurrence H, alkyl, cycloalkyl, heterocycloalkyl, aryl, heteroaryl, aralkyl, or heteroaralkyl;

or a compound of formula XXVII:

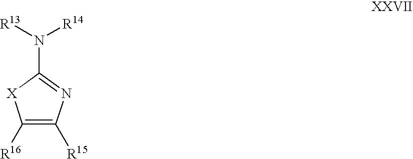

wherein,

X is O or S;

R¹³ is cycloalkenyl, heterocycloalkenyl, aryl, heteroaryl, aralkyl, heteroaralkyl, or —C(O)R¹⁷;

R¹⁴ is H or alkyl;

R¹⁵ is aryl;

R¹⁶ is H, alkyl, —CO₂R¹⁸, or —C(O)N(R¹⁸)₂;

R¹⁷ is cycloalkenyl, heterocycloalkenyl, heteroaryl, aralkyl, or heteroaralkyl; and

R¹⁸ represents independently for each occurrence H, alkyl, cycloalkyl, heterocycloalkyl, aryl, heteroaryl, aralkyl, or heteroaralkyl;

or a compound of formula XXVIII:

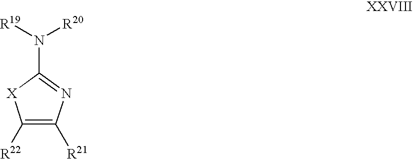

wherein,

X is O or S;

R¹⁹ is cycloalkenyl, heterocycloalkenyl, aryl, heteroaryl, aralkyl, heteroaralkyl, or —C(O)R²³;

R²⁰ is H or alkyl;

R²¹ is cycloalkenyl, heterocycloalkenyl, heteroaryl, aralkyl, heteroaralkyl, or an optionally substituted bicyclic ring having 1 or 2 heteroatoms selected form the group consisting of O, N, and S;

R²² is alkyl, —CO₂R⁸, or —C(O)N(R⁸)₂;

R²³ is cycloalkenyl, heterocycloalkenyl, heteroaryl, aralkyl, or heteroaralkyl; or R²³ is an aryl group optionally substituted with one or more of alkyl, halogen, —OR¹⁸, —N(R¹⁸)₂, —CO₂R¹⁸, C(O)N(R¹⁸)₂, cyano, or nitro; and

R¹⁸ represents independently for each occurrence H, alkyl, cycloalkyl, heterocycloalkyl, aryl, heteroaryl, aralkyl, or heteroaralkyl.

In certain embodiments, the present invention relates to the aforementioned compound having formula XXV, wherein X is S, R¹ is —C(O)R⁵, R² is H, and R⁴ is —CO₂R⁶.

In certain embodiments, the present invention relates to the aforementioned compound having formula XXVI, wherein X is S, R⁷ is —C(O)R¹¹, and R⁸ is H.

In certain embodiments, the present invention relates to the aforementioned compound having formula XXVII, wherein X is S, R¹³ is —C(O)R¹⁷, R¹⁴ is H, and R¹⁶ is —CO₂R¹⁸.

Another aspect of the invention relates to a compound of formula XXIX:

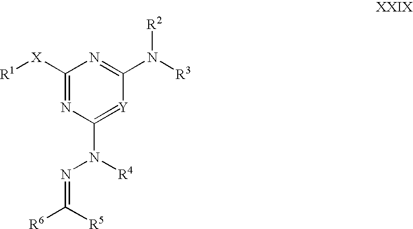

wherein,

X is O;

Y is —C(R⁸)—;

R¹ and R² represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R³ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R² and R³ taken together form a 3-8 member ring optionally substituted with one or more of alkyl, halogen, hydroxy, alkoxy, or amino;

R⁴ is H, alkyl, cycloalkyl, aryl, or aralkyl;

R⁵ is cycloalkyl, heterocycloalkyl, cycloalkenyl, heterocycloalkenyl, aryl, heteroaryl, aralkyl, heteroaralkyl, —(CR⁹═CR⁹)ₙ-aryl, or —(CR⁹═CR⁹)ₙ-heteroaryl;

R⁶ is H or alkyl; or R⁵ and R⁶ taken together form a optionally substituted monocyclic or bicyclic ring having 1 or 2 heteroatoms selected from the group consisting of O, N, and S;

R⁸ and R⁹ represent independently for each occurrence H or alkyl; and

n is 1 or 2;

or a compound represented by formula XXX:

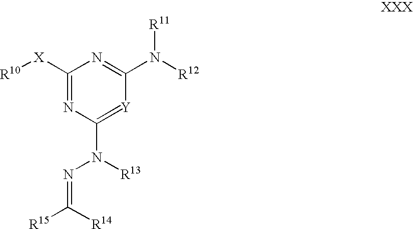

wherein,

X is —N(R¹⁶)—;

Y is —C(R¹⁷)—;

R¹⁰ and R¹¹ represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R¹² is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹¹ and R¹² taken together form a 3-8 member ring optionally substituted with one or more of alkyl, halogen, hydroxy, alkoxy, or amino;

R¹³ is H, alkyl, cycloalkyl, aryl, or aralkyl;

R¹⁴ is cycloalkyl, heterocycloalkyl, cycloalkenyl, heterocycloalkenyl, aryl, aralkyl, heteroaralkyl, —(CR¹⁸═CR¹⁸)ₙ-aryl, or —(CR¹⁸═CR¹⁸)ₙ-heteroaryl;

R¹⁵ is H or alkyl; or R¹⁴ and R¹⁵ taken together form a optionally substituted monocyclic or bicyclic ring having 1 or 2 heteroatoms selected from the group consisting of O, N, and S;

R¹⁶ is hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹⁰ and R¹⁶ taken together form a 3-8 member ring optionally substituted with one or more of alkyl, halogen, hydroxy, alkoxy, or amino;

R¹⁷ and R¹⁸ represent independently for each occurrence H or alkyl; and

n is 1 or 2.

In certain embodiments, the present invention relates to the aforementioned compound having formula XXX, wherein R¹⁶, R¹⁰, R¹¹, and R¹² are alkyl.

In certain embodiments, the present invention relates to the aforementioned compound0 having formula XXX, wherein R¹⁶, R¹⁰, R¹¹, and R¹² are alkyl, and R¹³ is,

In certain embodiments, the present invention relates to the aforementioned compound having formula XXX, wherein R¹⁶, R¹⁰, R¹¹, and R¹² are alkyl, R¹³ is H, and R¹⁴ is aryl.

Another aspect of the invention relates to a compound represented by formula XXXI:

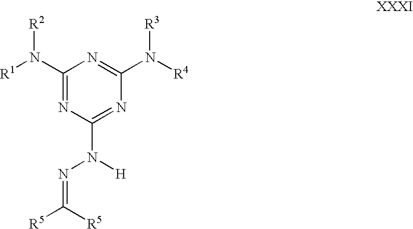

wherein,

R¹ and R³ represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R² and R⁴ represent independently hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R¹ and R² taken together form a 3-8 member ring; or R³ and R⁴ taken together form a 3-8 member ring; and

R⁵ represents independently cycloalkyl, aryl, heteroaryl, aralkyl, or heteraralkyl; or a compound represented by formula XXXII:

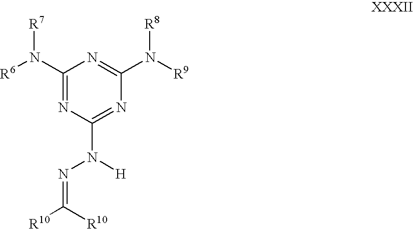

wherein,

R⁶ and R⁸ represent independently alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R⁷ and R⁹ represent independently hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; or R⁶ and R⁷ taken together form a 3-8 member ring; or R⁸ and R⁹ taken together form a 3-8 member ring; and

R¹⁰ represents independently hydrogen, alkyl, cycloalkyl, heteroaryl, aralkyl, or heteroaralkyl.

In certain embodiments, the present invention relates to the aforementioned compound having formula XXXI, wherein R¹ and R² taken together form a 7 membered ring, and R³ and R⁴ taken together form a 7 membered ring.

In certain embodiments, the present invention relates to the aforementioned compound having formula XXXI, wherein R¹ and R² taken together form a 7 membered ring, R³ and R⁴ taken together form a 7 membered ring, and R⁵ is aryl.

In certain embodiments, the present invention relates to the aforementioned compound having formula XXXII, wherein R¹ and R² taken together form a 7 membered ring, R³ and R⁴ taken together form a 7 membered ring, and R⁵ is alkyl.

In certain embodiments, the present invention relates to the aforementioned compound having formula XXXI, wherein R¹ and R³ are aryl.

In certain embodiments, the present invention relates to the aforementioned compound having formula XXXI, wherein R¹, R³, and R⁵ are aryl.

Another aspect of the invention relates to a compound represented by formula XXXIII:

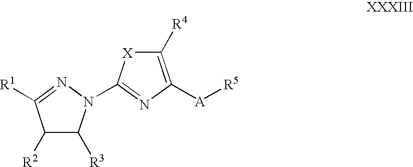

wherein,

X is O;

R¹, R³, and A represent independently cycloalkenyl, heterocycloalkenyl, aryl, heteroaryl, aralkyl, or heteroaralkyl;

R² and R⁴ represent independently H or alkyl;

R⁵ is an optionally substituted monocyclic or bicyclic ring having 1, 2, or 3 heteroatoms selected from the group consisting of O, N, and S;

or a compound of formula XXXIV:

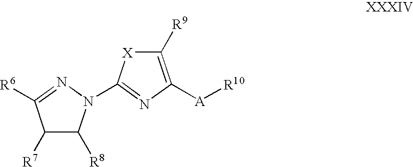

wherein,

X is S;

R⁶ represents cycloalkenyl, heterocycloalkenyl, heteroaryl, aralkyl, or heteroaralkyl;

R⁸ and A represent independently cycloalkenyl, heterocycloalkenyl, aryl, heteroaryl, aralkyl, or heteroaralkyl;

R⁷ and R⁹ represent independently H or alkyl;

R¹⁰ is an optionally substituted monocyclic or bicyclic ring having 1, 2, or 3 heteroatoms selected from the group consisting of O, N, and S;

or a compound of formula XXXV:

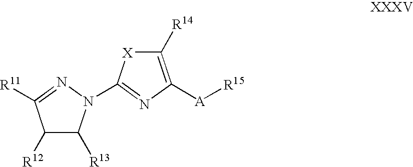

wherein,

X is S;

R¹¹ and A represent independently cycloalkenyl, heterocycloalkenyl, aryl, heteroaryl, aralkyl, or heteroaralkyl;

R¹³ represents cycloalkenyl, heterocycloalkenyl, heteroaryl, aralkyl, or heteroaralkyl;

R¹² and R¹⁴ represent independently H or alkyl;

R¹⁵ is an optionally substituted monocyclic or bicyclic ring having 1, 2, or 3 heteroatoms selected from the group consisting of O, N, and S;

or a compound of formula XXXVI:

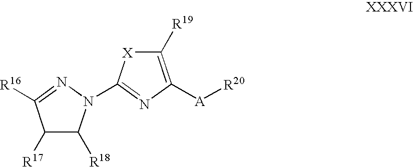

wherein,

X is S;

R¹⁶ and R¹⁸ represent independently cycloalkenyl, heterocycloalkenyl, aryl, heteroaryl, aralkyl, or heteroaralkyl;

A represents cycloalkenyl, heterocycloalkenyl, heteroaryl, aralkyl, or heteroaralkyl;

R¹⁷ and R¹⁹ represent independently H or alkyl;

R²⁰ is an optionally substituted monocyclic or bicyclic ring having 1, 2, or 3 heteroatoms selected from the group consisting of O, N, and S.

In certain embodiments, the present invention relates to the aforementioned compound having formula XXXIV, wherein X is S, A is aryl, R¹⁰ is a bicyclic ring having 1, 2, or 3 heteroatoms selected from the group consisting of O, N, and S; and R⁸ is aryl.

In certain embodiments, the present invention relates to the aforementioned compound having formula XXXV, wherein X is S; A is aryl; R¹⁵ is a bicyclic ring having 1, 2, or 3 heteroatoms selected from the group consisting of O, N, and S; and R¹¹ is aryl.

In certain embodiments, the present invention relates to the aforementioned compound having formula XXXVI, wherein X is S; R²⁰ is a bicyclic ring having 1, 2, or 3 heteroatoms selected from the group consisting of O, N, and S; R¹⁸ is aryl; and R¹⁶ is aryl.

Another aspect of the invention relates to a compound represented by formula XXXVII:

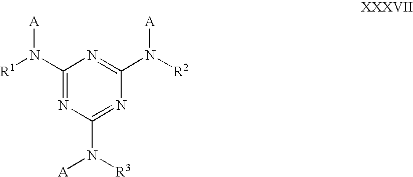

wherein,

R¹, R², and R³ are independently H, alkyl, heteroaryl, aralkyl, or heteroaralkyl; and

A is independently a mono or bicyclic aryl or heteroaryl, substituted with halide, alkyl, nitro, amino, aryl, heteroaryl, aralkyl, heteroaralkyl, cycloalkyl, or heterocycloalkyl.

In certain embodiments, the present invention relates to the aforementioned compound, wherein R¹, R², and R³ are H.

In certain embodiments, the present invention relates to the aforementioned compound, wherein R¹, R², and R³ are H; and A is a monocyclic aryl substituted with an amino.

In certain embodiments, the present invention relates to the aforementioned compound, wherein R¹, R², and R³ are H; and A is a monocyclic aryl substituted with a nitro.

In certain embodiments, the present invention relates to the aforementioned compound, wherein R¹, R², and R³ are H; and A is a monocyclic aryl substituted with a halide.

Another aspect of the invention relates to a compound represented by formula XXXVIII:

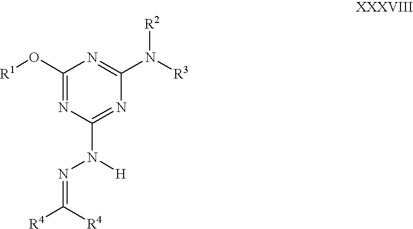

wherein,

R¹ represents alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl;

R² and R³ represent independently hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; and

R⁴ represents independently hydrogen, alkyl, cycloalkyl, heteroaryl, aralkyl, or heteroaralkyl;

or a compound of formula XXXIX:

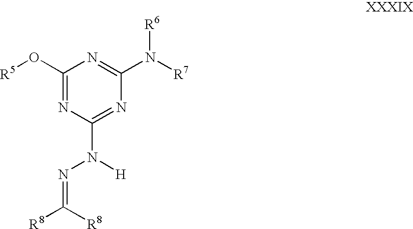

wherein,

R⁵ represents alkyl, heteroalkyl, cycloalkyl, heterocycloalkyl, alkenyl, cycloalkenyl, heterocycloalkenyl, aryl, heteroaryl, aralkyl, or heteroaralkyl;

R⁶ and R⁷ represent independently hydrogen, alkyl, heteroalkyl, haloalkyl, cycloalkyl, heterocycloalkyl, alkenyl, cycloalkenyl, heterocycloalkenyl, aryl, heteroaryl, aralkyl, or heteroaralkyl; and

R⁸ represents independently hydrogen, alkyl, cycloalkyl, aryl, heteroaryl, aralkyl, or heteroaralkyl.

In certain embodiments, the present invention relates to the aforementioned compound having formula XXXVIII, wherein R¹ is haloalkyl.

In certain embodiments, the present invention relates to the aforementioned compound having formula XXXVIII, wherein R¹ is haloalkyl, and R² and R³ are aryl.

In certain embodiments, the present invention relates to the aforementioned compound having formula XXXVIII, wherein R¹ is haloalkyl; R² and R³ are aryl; and at least one R⁴ is hydrogen.

In certain embodiments, the present invention relates to the aforementioned compound having formula XXXIX, wherein R⁶ and R⁷ are aryl.

In certain embodiments, the present invention relates to the aforementioned compound having formula XXXIX, wherein R⁶ and R⁷ are aryl; and at least one R⁸ is aryl.

In certain embodiments, the present invention relates to the aforementioned compound having formula XXXIX, wherein R⁶ and R⁷ are aryl; one R⁸ is aryl; and one R⁸ is hydrogen.

Another aspect of the present invention relates to a pharmaceutical composition comprising a pharmaceutically acceptable excipient and a compound of any one of formulae X-XXXIX, wherein formulae X-XXXIX are as described above.

### DEFINITIONS

For convenience, certain terms employed in the specification, examples, and appended claims are collected here.

The term “ACAT” refers to acyl co-A: cholesterol acyl transferase.

The term “CHO” refers to Chinese hamster ovary.

The term “DMSO” refers to dimethyl sulfoxide;

The term “FBS” refers to fetal bovine serum.

The term “GC” refers to gas chromatography.

The term “HEPES” refers to 4-(2-hydroxyethyl)-1-pipiperazine ethane sulphonic acid.

The term “LBPA” refers to lyso-bis phosphatidic acid.

The term “LDL” refers to low density lipoprotein.

The term “LSO” refers to lysosomal storage organelles.

The term “NPC” refers to Niemann-Pick disease type C.

The term “PBS” refers to phosphate buffered saline.

The term “PFA” refers to para-formaldehyde.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroalkyl” is art-recognized, and includes saturated aliphatic groups containing at least one heteroatom in the chain, including straight-chain alkyl groups containing at least one heteroatom in the chain, branched-chain alkyl groups containing at least one heteroatom in the chain, cycloalkyl (alicyclic) groups containing at least one heteroatom in the ring, alkyl substituted cycloalkyl groups containing at least one heteroatom in the ring, and cycloalkyl substituted alkyl groups containing at least one heteroatom in the chain. The term “heterocycloalkyl” refers to a cycloalkyl (alicyclic) group containing at least one heteroatom in the ring.

The terms “alkenyl” and “alkynyl” are art-recognized and refer to unsaturated aliphatic groups analogous in length to the alkyl groups described above, but contain at least one double or triple bond, respectively. The terms “alkenyl” and “alkynyl” are meant to include unsubstituted unsaturated aliphatic groups as well as unsaturated aliphatic groups containing one or more substituents selected from the group consisting of halogen, alkyl, alkoxyl, carbonyl, and carboxyl.

The term “cycloalkenyl” refers to an alicyclic group containing least one double bond. The term “cycloalkenyl” is meant to include unsubstituted unsaturated alicyclic groups as well as unsaturated alicyclic groups containing one or more substituents selected from the group consisting of halogen, alkyl, alkoxyl, carbonyl, and carboxyl.

The term “heterocycloalkenyl” refers to an alicyclic group containing least one double bond and at least one heteroatom selected from the group consisting of N, O, and S. The term “heterocycloalkenyl” is meant to include unsubstituted unsaturated alicyclic groups as well as unsaturated alicyclic groups containing one or more substituents selected from the group consisting of halogen, alkyl, alkoxyl, carbonyl, and carboxyl.

The term “aryl” is art-recognized and refers to 5-, 6- and 7-membered single-ring aromatic groups where the ring structure is formed from carbon atoms, for example, benzene, naphthalene, anthracene, pyrene, and the like. The aromatic ring may be substituted at one or more ring positions with a substituent. Representative substituents include halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryl, heteroaryl, and/or heterocyclyls.

The term “heteroaryl” is art-recognized and refers to 5-, 6- and 7-membered single-ring aromatic groups that have one to four heteroatoms in the ring, for example, pyrrole, furan, thiophene, imidazole, oxazole, thiazole, triazole, pyrazole, pyridine, pyrazine, pyridazine and pyrimidine, and the like. Those aromatic groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “heteroaryl” also includes polycyclic ring systems having two or more cyclic rings in which two or more carbons are common to two adjoining rings (the rings are “fused rings”) wherein at least one of the rings is heteroaryl, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The term “aralkyl” is art-recognized and refers to an alkyl group substituted with an aryl group.

The term “heteroaralkyl” is art-recognized and refers to an alkyl group substituted with a heteroaryl group.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like. The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

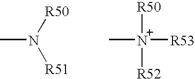

wherein R50, R51 and R52 each independently represent a hydrogen, an alkyl, an alkenyl, —(CH₂)ₘR61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group. The term “acylamino” is art-recognized and refers to a moiety that may be represented by the general formula:

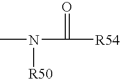

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

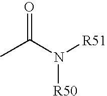

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

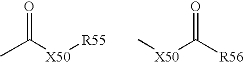

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

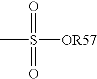

in which R57 is an electron pair, hydrogen, alkyl, cycloalkyl, or aryl.

The term “sulfate” is art recognized and includes a moiety that may be represented by the general formula:

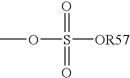

in which R57 is as defined above.

The term “sulfonamido” is art recognized and includes a moiety that may be represented by the general formula:

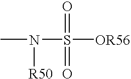

in which R50 and R56 are as defined above.

The term “sulfamoyl” is art-recognized and refers to a moiety that may be represented by the general formula:

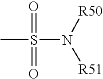

in which R50 and R51 are as defined above.

The term “sulfonyl” is art-recognized and refers to a moiety that may be represented by the general formula:

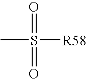

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

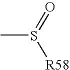

in which R58 is defined above.

The term “phosphoryl” is art-recognized and may in general be represented by the formula:

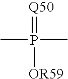

wherein Q50 represents S or O, and R59 represents hydrogen, a lower alkyl or an aryl. When used to substitute, e.g., an alkyl, the phosphoryl group of the phosphorylalkyl may be represented by the general formulas:

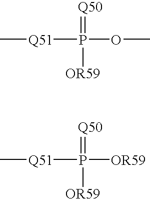

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

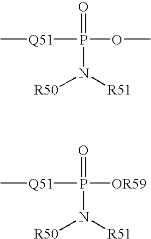

wherein Q51, R50, R51 and R59 are as defined above.

The term “phosphonamidite” is art-recognized and may be represented in the general formulas:

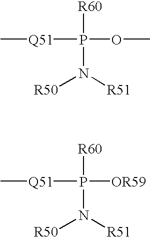

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P.G.M. Protective Groups in Organic Synthesis, 2^{nd} ed.; Wiley: New York, 1991). Protected forms of the inventive compounds are included within the scope of this invention.

For purposes of this invention, the chemical elements are identified in accordance with the Periodic Table of the Elements, CAS version, Handbook of Chemistry and Physics, 67-th Ed., 1986-87, inside cover.

Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

Micelles

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Polymers

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins

Cyclodextrins are cyclic oligosaccharides, consisting of 6, 7 or 8 glucose units, designated by the Greek letter alpha, beta, or gamma, respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17-beta-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility. Numerous cyclodextrins and methods for their preparation have been described. For example, Parmeter (I), et al. (U.S. Pat. No. 3,453,259, hereby incorporated herein by reference) and Gramera, et al. (U.S. Pat. No. 3,459,731, hereby incorporated herein by reference) described electroneutral cyclodextrins. Other derivatives include cyclodextrins with cationic properties [Parmeter (II), U.S. Pat. No. 3,453,257, hereby incorporated herein by reference], insoluble crosslinked cyclodextrins (Solms, U.S. Pat. No. 3,420,788, hereby incorporated herein by reference), and cyclodextrins with anionic properties [Parmeter (III), U.S. Pat. No. 3,426,011, hereby incorporated herein by reference]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127, hereby incorporated herein by reference).

Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing a compound of the present invention, where the liposome membrane is formulated to provide a liposome with increased carrying capacity. Alternatively or in addition, the compound of the present invention may be contained within, or adsorbed onto, the liposome bilayer of the liposome. The compound of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C.sub.14 to about C.sub.20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

Liposomes according to the present invention may be prepared by any of a variety of techniques that are known in the art. See, e.g., U.S. Pat. No. 4,235,871; Published PCT applications WO 96/14057, both of which are hereby incorporated herein by reference; New RRC, Liposomes: A practical approach, IRL Press, Oxford (1990), pages 33-104; Lasic D D, Liposomes from physics to applications, Elsevier Science Publishers BV, Amsterdam, 1993.

For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, hereby incorporated herein by reference

Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

Combinatorial Libraries

The subject compounds may be synthesized using the methods of combinatorial synthesis described in this section. Combinatorial libraries of the compounds may be used for the screening of pharmaceutical, agrochemical or other biological or medically-related activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051, each of which is hereby incorporated herein by reference; Chen et al. (1994) JACS 116:2661: Kerr et al. (1993) JACS 115:252; PCT publications WO92/10092, WO93/09668 and WO91/07087; and the Lerner et al. PCT publication WO93/20242, each of which is hereby incorporated herein by reference). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, which is particularly suitable for discovering enzyme inhibitors, the beads can be dispersed on the surface of a permeable membrane, and the diversomers released from the beads by lysis of the bead linker. The diversomer from each bead will diffuse across the membrane to an assay zone, where it will interact with an enzyme assay. Detailed descriptions of a number of combinatorial methodologies are provided below.

A. Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

B. Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

C. Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971, each of which is hereby incorporated herein by reference). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

D. Combinatorial Libraries by Light-Directed Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854, hereby incorporated herein by reference; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

E. Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1. Tagging with Sequenceable Bio-Oligomers The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2. Non-sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded

### REFERENCES

1. Pentchev, P., Vanier, M T., Suzuki, K., and Patterson, M C. 1995. Niemann-Pick Disease type C: Cellular cholesterol lipidosis. New York: McGraw-Hill. 2625-2639 pp.
2. Li, H., Repa, J. J., Valasek, M. A., Beltroy, E. P., Turley, S. D., German, D. C., and Dietschy, J. M. 2005. Molecular, anatomical, and biochemical events associated with neurodegeneration in mice with Niemann-Pick type C disease. J Neuropathol Exp Neurol 64:323-333.
3. Patterson, M. C., and Platt, F. 2004. Therapy of Niemann-Pick disease, type C. Biochim Biophys Acta 1685:77-82.
4. Hsich, G., Sena-Esteves, M., and Breakefield, X. O. 2002. Critical issues in gene therapy for neurologic disease. Hum Gene Ther 13:579-604.
5. Liscum, L., Arnio, E., Anthony, M., Howley, A., Sturley, S. L., and Agler, M.
2002. Identification of a pharmaceutical compound that partially corrects the Niemann-Pick C phenotype in cultured cells. J Lipid Res 43:1708-1717.
6. Bascunan-Castillo, E. C., Erickson, R. P., Howison, C. M., Hunter, R. J., Heidenreich, R. H., Hicks, C., Trouard, T. P., and Gillies, R. J. 2004. Tamoxifen and vitamin E treatments delay symptoms in the mouse model of Niemann-Pick C. J Appl Genet. 45:461-467.
7. Zervas, M., Somers, K. L., Thrall, M. A., and Walkley, S. U. 2001. Critical role for glycosphingolipids in Niemann-Pick disease type C. Curr Biol 11: 1283-1287.
8. Griffin, L. D., Gong, W., Verot, L., and Mellon, S. H. 2004. Niemann-Pick type C disease involves disrupted neurosteroidogenesis and responds to allopregnanolone. Nat Med 10:704-711.
9. Vanier, M. T., and Suzuki, K. 1998. Recent advances in elucidating Niemann-Pick C disease. Brain Pathol 8:163-174.
10. Goldstein, J. L., and Brown, M. S. 1992. Lipoprotein receptors and the control of plasma LDL cholesterol levels. Eur Heart J 13 Suppl B:34-36.
11. Garver, W. S., and Heidenreich, R. A. 2002. The Niemann-Pick C proteins and trafficking of cholesterol through the late endosomal/lysosomal system. Curr Mol Med 2:485-505.
12. Carstea, E. D., Morris, J. A., Coleman, K. G., Loftus, S. K., Zhang, D., Cummings, C., Gu, J., Rosenfeld, M. A., Pavan, W. J., Krizman, D. B., et al. 1997. Niemann-Pick Cl disease gene: homology to mediators of cholesterol homeostasis. Science 277:228-231.
13. Liscum, L. 2000. Niemann-Pick type C mutations cause lipid traffic jam. Traffic 1:218-225.
14. Puri, V., Watanabe, R., Dominguez, M., Sun, X., Wheatley, C. L., Marks, D. L., and Pagano, R. E. 1999. Cholesterol modulates membrane traffic along the endocytic pathway in sphingolipid-storage diseases. Nat Cell Biol 1:386-388.
15. Ioannou, Y. A. 2000. The structure and function of the Niemann-Pick C1 protein. Mol Genet Metab 71:175-181.
16. Scott, C., and Ioannou, Y. A. 2004. The NPC1 protein: structure implies function. Biochim Biophys Acta 1685:8-13.
17. Vanier, M. T., and Millat, G. 2004. Structure and function of the NPC2 protein. Biochim Biophys Acta 1685:14-21.
18. Friedland, N., Liou, H. L., Lobel, P., and Stock, A. M. 2003. Structure of a cholesterol-binding protein deficient in Niemann-Pick type C2 disease. Proc Natl Acad Sci USA 100:2512-2517.
19. Okamura, N., Kiuchi, S., Tamba, M., Kashima, T., Hiramoto, S., Baba, T., Dacheux, F., Dacheux, J. L., Sugita, Y., and Jin, Y. Z. 1999. A porcine homolog of the major secretory protein of human epididymis, HE1, specifically binds cholesterol. Biochim Biophys Acta 1438:377-387.
20. Zhang, M., Sun, M., Dwyer, N. K., Comly, M. E., Patel, S. C., Sundaram, R., Hanover, J. A., and Blanchette-Mackie, E. J. 2003. Differential trafficking of the Niemann-Pick C1 and 2 proteins highlights distinct roles in late endocytic lipid trafficking. Acta Paediatr Suppl 92:63-73; discussion 45.
21. Sun, X., Marks, D. L., Park, W. D., Wheatley, C. L., Puri, V., O'Brien, J. F., Kraft, D. L., Lundquist, P. A., Patterson, M. C., Pagano, R. E., et al. 2001. Niemann-Pick C variant detection by altered sphingolipid trafficking and correlation with mutations within a specific domain of NPC1. Am J Hum Genet. 68:1361-1372.
22. Kobayashi, T., Beuchat, M. H., Lindsay, M., Frias, S., Palmiter, R. D., Sakuraba, H., Parton, R. G., and Gruenberg, J. 1999. Late endosomal membranes rich in lysobisphosphatidic acid regulate cholesterol transport. Nat Cell Biol 1:113-118.
23. Mukherjee, S., and Maxfield, F. R. 2004. Lipid and cholesterol trafficking in NPC. Biochim Biophys Acta 1685:28-37.
24. Bornig, H., and Geyer, G. 1974. Staining of cholesterol with the fluorescent antibiotic “filipin”. Acta Histochem 50:110-115.
25. Maxfield, F. R., and Wustner, D. 2002. Intracellular cholesterol transport. J Clin Invest 110:891-898.
26. Lange, Y., Ye, J., and Steck, T. L. 1998. Circulation of cholesterol between lysosomes and the plasma membrane. J Biol Chem 273:18915-18922.
27. Chen, W., Sun, Y., Welch, C., Gorelik, A., Leventhal, A. R., Tabas, I., and Tall, A. R. 2001. Preferential ATP-binding cassette transporter A1-mediated cholesterol efflux from late endosomes/lysosomes. J Biol Chem 276:43564-43569.
28. Pentchev, P. G., Comly, M. E., Kruth, H. S., Vanier, M. T., Wenger, D. A., Patel, S., and Brady, R. O. 1985. A defect in cholesterol esterification in Niemann-Pick disease (type C) patients. Proc Natl Acad Sci USA 82:8247-8251.
29. Lin, S., Lu, X., Chang, C. C., and Chang, T. Y. 2003. Human acyl-coenzyme A:cholesterol acyltransferase expressed in chinese hamster ovary cells: membrane topology and active site location. Mol Biol Cell 14:2447-2460.
30. Park, W. D., O'Brien, J. F., Lundquist, P. A., Kraft, D. L., Vockley, C. W., Karnes, P. S., Patterson, M. C., and Snow, K. 2003. Identification of 58 novel mutations in Niemann-Pick disease type C: correlation with biochemical phenotype and importance of PTC1-like domains in NPC1. Hum Mutat 22:313-325.
31. Yang, C. C., Su, Y. N., Chiou, P. C., Fietz, M. J., Yu, C. L., Hwu, W. L., and Lee, M. J. 2005. Six novel NPC1 mutations in Chinese patients with Niemann-Pick disease type C. J Neurol Neurosurg Psychiatry 76:592-595.
32. Vanier, M. T., and Millat, G. 2003. Niemann-Pick disease type C. Clin Genet. 64:269-281.
33. Di Leo, E., Panico, F., Tarugi, P., Battisti, C., Federico, A., and Calandra, S. 2004. A point mutation in the lariat branch point of intron 6 of NPC1 as the cause of abnormal pre-mRNA splicing in Niemann-Pick type C disease. Hum Mutat 24:440.
34. Patterson, M. C., Vanier, M. T., Suzuki, K, Morris, J. A., Cartsea, E., Neufeld, E. B., Blanchette-Mackie, E. J., Pentchev, P. G. 2001. Niemann-Pick type C: a lipid trafficking disorder. In The metabolic and molecular bases of inherited disease. B. A. Scriver S R, Valle D, Sly W S, Childs B, Kinzler K W, Vogelstein B, editor. New York: McGraw Hill. 3611-3633.
35. Meiner, V., Shpitzen, S., Mandel, H., Klar, A., Ben-Neriah, Z., Zlotogora, J., Sagi, M., Lossos, A., Bargal, R., Sury, V., et al. 2001. Clinical-biochemical correlation in molecularly characterized patients with Niemann-Pick type C. Genet Med 3:343-348.
36. Choudhury, A., Dominguez, M., Puri, V., Sharma, D. K., Narita, K., Wheatley, C. L., Marks, D. L., and Pagano, R. E. 2002. Rab proteins mediate Golgi transport of caveola-internalized glycosphingolipids and correct lipid trafficking in Niemann-Pick C cells. J Clin Invest 109:1541-1550.
37. Blom, T. S., Linder, M. D., Snow, K., Pihko, H., Hess, M. W., Jokitalo, E., Veckman, V., Syvanen, A. C., and Ikonen, E. 2003. Defective endocytic trafficking of NPC1 and NPC2 underlying infantile Niemann-Pick type C disease. Hum Mol Genet. 12:257-272.
38. Walter, M., Davies, J. P., and Ioannou, Y. A. 2003. Telomerase immortalization upregulates Rab9 expression and restores LDL cholesterol egress from Niemann-Pick Cl late endosomes. J Lipid Res 44:243-253.
39. Maxfield, F. R., and McGraw, T. E. 2004. Endocytic recycling. Nat Rev Mol Cell Biol 5:121-132.
40. Mukherjee, S., and Maxfield, F. R. 2004. Membrane domains. Annu Rev Cell Dev Biol 20:839-866.
41. Prinz, W. 2002. Cholesterol trafficking in the secretory and endocytic systems. Semin Cell Dev Biol 13:197-203.
42. Cruz, J. C., Sugii, S., Yu, C., and Chang, T. Y. 2000. Role of Niemann-Pick type C1 protein in intracellular trafficking of low density lipoprotein-derived cholesterol. J Biol Chem 275:4013-4021.
43. Shiratori, Y., Okwu, A. K., and Tabas, I. 1994. Free cholesterol loading of macrophages stimulates phosphatidylcholine biosynthesis and up-regulation of CTP: phosphocholine cytidylyltransferase. J Biol Chem 269:11337-11348.
44. Lowry, O. H., Rosebrough, N. J., Farr, A. L., and Randall, R. J. 1951. Protein measurement with the Folin phenol reagent. J Biol Chem 193:265-275.
45. McGraw, T. E., Greenfield, L., and Maxfield, F. R. 1987. Functional expression of the human transferrin receptor cDNA in Chinese hamster ovary cells deficient in endogenous transferrin receptor. J Cell Biol 105:207-214.
46. Zhang, J. H., Chung, T. D., and Oldenburg, K. R. 1999. A Simple Statistical Parameter for Use in Evaluation and Validation of High Throughput Screening Assays. J Biomol Screen 4:67-73.
47. Willet P., B., J M., Down, G M. 1998. Chemical Similarity Searching. J. Chem. Inf. Comput.Sci 38:983-996.
48. Zhang, M., Dwyer, N. K., Neufeld, E. B., Love, D. C., Cooney, A., Comly, M., Patel, S., Watari, H., Strauss, J. F., 3rd, Pentchev, P. G., et al. 2001. Sterol-modulated glycolipid sorting occurs in niemann-pick C1 late endosomes. J Biol Chem 276:3417-3425.
49. Boven, L. A., van Meurs, M., Boot, R. G., Mehta, A., Boon, L., Aerts, J. M., and Laman, J. D. 2004. Gaucher cells demonstrate a distinct macrophage phenotype and resemble alternatively activated macrophages. Am J Clin Pathol 122:359-369.
50. Platt, F. M., Neises, G. R., Reinkensmeier, G., Townsend, M. J., Perry, V. H., Proia, R. L., Winchester, B., Dwek, R. A., and Butters, T. D. 1997. Prevention of lysosomal storage in Tay-Sachs mice treated with N-butyldeoxynojirimycin. Science 276:428-431.
51. Neufeld, E. B., Stonik, J. A., Demosky, S. J., Jr., Knapper, C. L., Combs, C. A., Cooney, A., Comly, M., Dwyer, N., Blanchette-Mackie, J., Remaley, A. T., et al. 2004. The ABCA1 transporter modulates late endocytic trafficking: insights from the correction of the genetic defect in Tangier disease. J Biol Chem 279:15571-15578.
52. Brzozowski, Z. 1998. 2-Mercapto-N-(azolyl)benzenesulfonamides. V. Syntheses, anti-HIV and anticancer activity of some 4-chloro-2-mercapto-5-methyl-N-(1,2,4-triazolo[4,3-a]pyrid-3-yl)benzenesulfonamides. Acta Pol Pharm 55:375-379.
53. Brzozowski, Z., Saczewski, F., and Gdaniec, M. 2000. Synthesis, structural characterization and antitumor activity of novel 2,4-diamino-1,3,5-triazine derivatives. Eur J Med Chem 35:1053-1064.
54. Schatzberg, A. F. 2004. Employing pharmacologic treatment of bipolar disorder to greatest effect. J Clin Psychiatry 65 Suppl 15:15-20.
55. Mohr, R., Buschauer, A., and Schunack, W. 1986. [H2-antihistaminics. 31. 1,2,5-Triazine-2,4-diamines and -2,4,6-triamines with H2-antagonistic activity]. Arch Pharm (Weinheim) 319:878-885.
56. Chambers, M. S., Atack, J. R., Carling, R. W., Collinson, N., Cook, S. M., Dawson, G. R., Ferris, P., Hobbs, S. C., O'Connor, D., Marshall, G., et al. 2004. An orally bioavailable, functionally selective inverse agonist at the benzodiazepine site of GABAA alphas receptors with cognition enhancing properties. J Med Chem 47:5829-5832.
57. Hao, M., Lin, S. X., Karylowski, O. J., Wustner, D., McGraw, T. E., and Maxfield, F. R. 2002. Vesicular and non-vesicular sterol transport in living cells. The endocytic recycling compartment is a major sterol storage organelle. J Biol Chem 277:609-617.
58. Sleat, D. E., Wiseman, J. A., El-Banna, M., Price, S. M., Verot, L., Shen, M. M., Tint, G. S., Vanier, M. T., Walkley, S. U., and Lobel, P. 2004. Genetic evidence for nonredundant functional cooperativity between NPC1 and NPC2 in lipid transport. Proc Natl Acad Sci USA 101:5886-5891.
59. Strauss, J. F., 3rd, Kishida, T., Christenson, L. K., Fujimoto, T., and Hiroi, H. 2003. START domain proteins and the intracellular trafficking of cholesterol in steroidogenic cells. Mol Cell Endocrinol 202:59-65.
60. Tall, A. R. 2003. Role of ABCA1 in cellular cholesterol efflux and reverse cholesterol transport. Arterioscler Thromb Vasc Biol 23:710-711.
61. Choudhury, A., Sharma, D. K., Marks, D. L., and Pagano, R. E. 2004. Elevated endosomal cholesterol levels in Niemann-Pick cells inhibit rab4 and perturb membrane recycling. Mol Biol Cell 15:4500-4511.

### EXAMPLES

The invention now being generally described, it will be more readily understood by reference to the following examples, which are included merely for purposes of illustration of certain aspects and embodiments of the present invention, and are not intended to limit the invention.

### Example 1

### Filipin Binding Assay

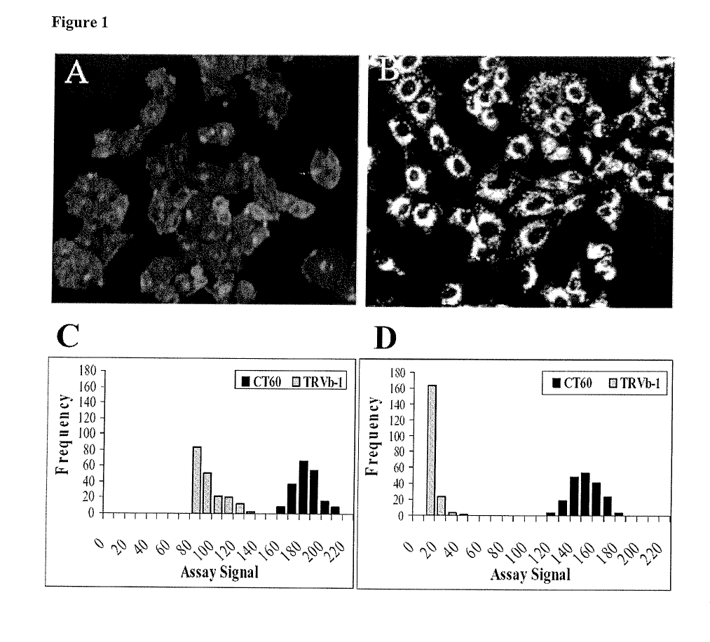

We developed assays for correction of the NPC phenotype in CHO cell lines using filipin, which binds to non-esterified cholesterol and has been used to visualize the free cholesterol content in NPC cells. The initial screens were carried out in CT60 cells, which express a mutated hamster NPC1 protein and a gain of function mutation in SCAP (42). These cells cannot traffic LDL-derived cholesterol out of late endosomes, and large amounts of cholesterol accumulate in LSO compartments. FIGS. 1A-B show images of filipin staining in a control CHO cell line, TRVb1 (45) (FIG. 1A) and in CT60 cells (FIG. 1B). It can be seen that the CT60 cells show much more filipin staining than control cells and that the fluorescence in CT60 cells is concentrated in peri-nuclear organelles.

For screening, images were acquired using a Discovery-1 automated microscopy system with a 10× objective and corrected for background and shading as described in Methods. Two thresholds were set for filipin staining, a low threshold to include all cell areas, and a high threshold for the strong filipin staining in the peri-nuclear LSOs. The threshold values were set for each plate from analysis of 64 images in 32 wells of untreated CT60 cells. It was found that the same thresholds could be used for multiple plates within an experiment, but the thresholds would vary among experiments conducted on different days. In developing the low threshold, we compared the outlines based on the thresholded cell images with transmitted light images, and the thresholds provided good agreement with the transmitted light cell boundaries.

As a simple measure of the intensity of filipin staining, we measured the filipin intensity per pixel above the threshold. The conditions of filipin labeling (time and concentration) were adjusted to optimize the discrimination between the CT60 cells and control TRVb1 cells. As shown in FIG. 1C, this simple method provided a reasonably high degree of discrimination between the CT60 cells and TRVb1 cells. The quality of the assay for screening is expressed in terms of a statistical parameter Z′(46):

Z′=1−(3σ_{c+}+3σ_{c-})/|μ_{c+}−μ_{c-}|

where σ_{c+} and σ_{c-} are the standard deviations (SD) of the positive and negative control data sets and μ_{c+} and μ_{c-} are the mean values of the positive and negative controls. The Z′ value as calculated by using average filipin intensity for the CT60 versus TRVb1 cells was 0.22, which is generally considered to be inadequate for large scale screening because of the expected overlap of the two distributions when large numbers of wells are screened.

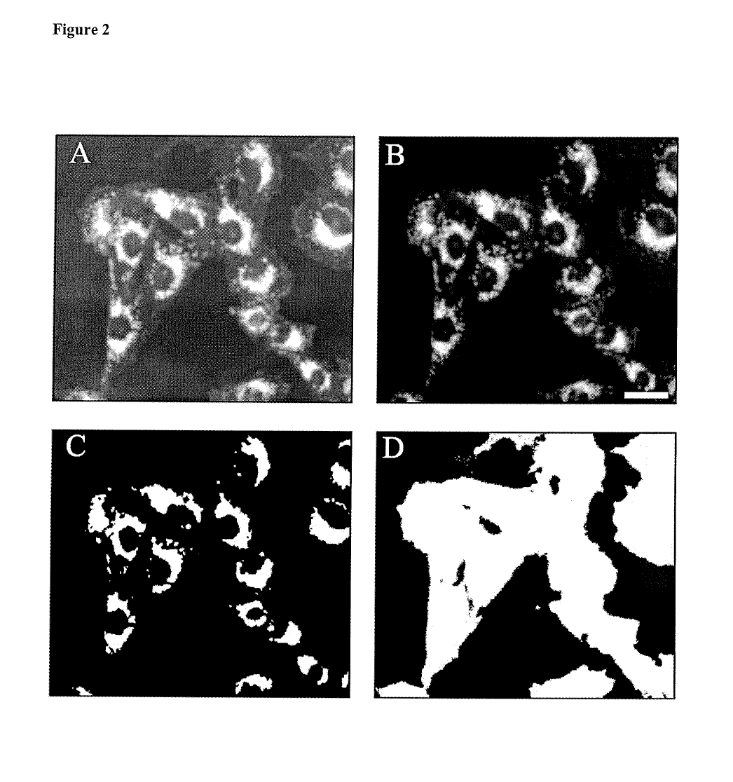

Although the intensity assay was potentially useful, it seemed that additional information in the images could provide better discrimination of wild type vs. NPC cells. For this purpose, we took advantage of the spatial distribution of the filipin labeling of the LSOs, which cluster near the nucleus (FIG. 1B). We applied the second, higher threshold for this bright cluster. An example of the application of these thresholds is shown in FIG. 2. We then measured the total fluorescence intensity in the selected LSO objects (high threshold, FIG. 2C) divided by the total number of pixels in cells (low threshold, FIG. 2D). As shown in FIG. 1D, this LSO compartment assay gave a much stronger discrimination of the CT60 versus the control cells with a Z′ value of 0.61.

Using the above screening technique, we screened a library of 14,956 compounds added to cells for 16 hours at a final concentration of 10 μM, using a single well for each compound. Prior to application of the compounds, cells were grown in normal tissue culture medium containing 10% FBS, so the LSOs were filled with cholesterol (FIG. 1B). The cells were then imaged and analyzed using the average filipin intensity assay and the LSO compartment assay. In general, the two analyses identified similar compounds that reduced the filipin labeling.

Wells that had average filipin intensities more than 3 SD from the mean value of solvent treated cells were then examined further. Images from these selected wells were inspected visually, and sites that showed poor focus (about 0.3% of the total) were not re-analyzed if we had images from the second position. Wells that had a low number of cells were taken as an indication of toxicity, and these cytotoxic compounds were not pursued further. We also examined arrayed low magnification images of rows and columns from the plates to look for patterns of cell number or brightness that might indicate a mechanical error in one of the automated pipetting steps. In one case, a banding pattern was seen in one set of 8 plates, and these compounds were re-screened after fixing the pipetting problem.

From this initial screen, we found 133 compounds that reduced the average filipin intensity by more than 3 SD and 23 compounds that increased the average filipin intensity by more than 3 SD. Visual inspection of images also revealed 19 compounds that produced morphological changes in the filipin staining pattern without meeting our criteria for reducing average filipin intensity.

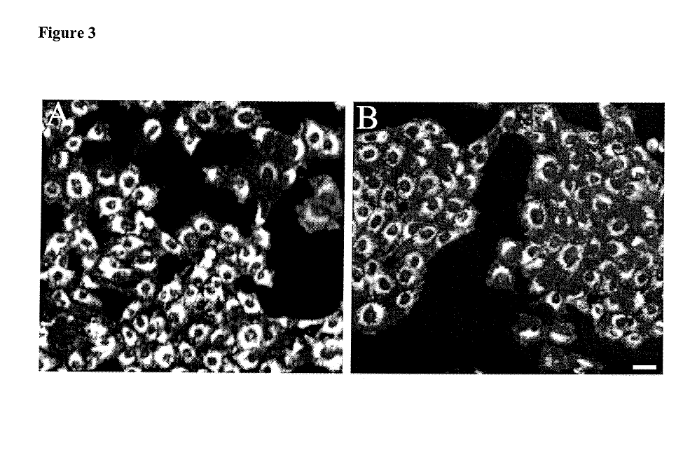

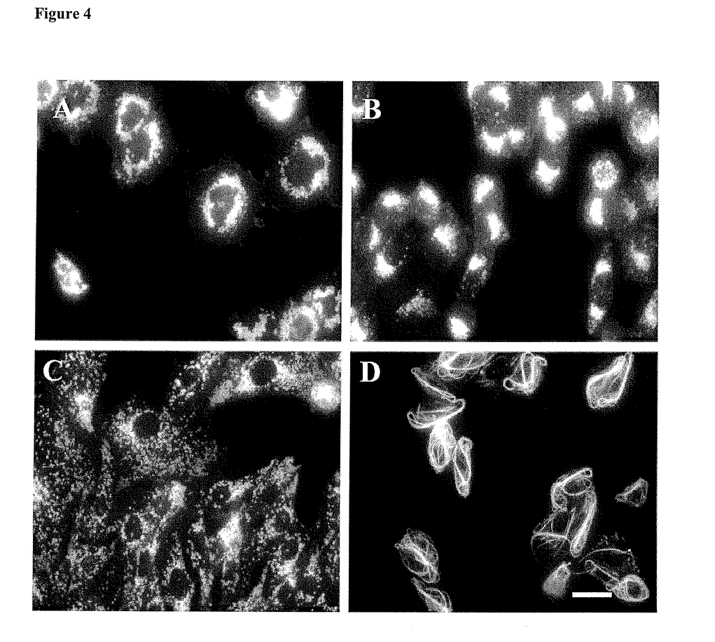

These 175 compounds were then re-screened at 10 μM under the same conditions as the initial screen, except that each compound was placed in two wells per plate and duplicate plates were screened in parallel. Both the average filipin intensity and the LSO ratio values were determined. From the re-screening 14 compounds were selected that reproducibly reduced filipin staining at 10 μM, and 8 compounds were found to reproducibly increase filipin staining. Nine compounds were found to alter the morphological distribution of filipin. FIG. 3 shows screening plate images of solvent treated control well and compound treated well showing decreased filipin staining.

FIG. 4 shows the effect of four compounds that caused morphological changes as observed by filipin staining. These cells show rearrangements that may indicate that cholesterol has been redirected to a different compartment or that the morphology of the LSOs themselves has been changed (compound 1-c-3, FIG. 4C). The effect of compound 1-b-4 (FIG. 4D) was extremely dramatic as observed by bright swirls of filipin staining. The effects of compound 1-b-4 were similar in normal human fibroblasts (data not shown), indicating that this response was not related to the NPC phenotype of the cells. The compounds that increased the apparent filipin staining and those that caused morphological changes were not investigated further in this study.

The structures of 14 compounds that decreased filipin labeling and two that caused significant morphological changes are shown in FIG. 5. Compounds 1-c-2 and 1-c-3 caused morphological changes and compounds 1-b-2 and 1-b-4 increased filipin intensity.

Materials and Methods for Screening Assay

Materials: Cell growth medium Hams F12 and fetal bovine serum (FBS) were purchased from Invitrogen Corporation (Carlsbad, Calif.). All other chemicals, including dimethyl sulfoxide (DMSO), filipin, paraformaldehyde (PFA) and Hoechst 33258, were purchased from Sigma Chemicals (St. Louis, Mo.). The compound library for screening was purchased from Chemical Diversity, Inc. (San Diego, Calif.). Metamorph image analysis software was from Molecular Devices Corporation (Downington, Pa.).

Cell Culture The NPC1 cell lines CT60 and CT43 were provided by T. Y. Chang (Dartmouth Medical School, Hanover, N.H.). These cell lines are derived from the parental cell line, 25RA, which is a CHO cell line containing a gain of function mutation in the SREBP cleavage-activating protein (SCAP) (42). Both CT60 and CT43 cells were grown in Hams F12 supplemented with 1% Penicillin/Streptomycin (PS), 2 g/L glucose, 1.176 g/L sodium bicarbonate [Medium A] containing 10% FBS in a humidified incubator with 5% CO₂ maintained at 37° C. For screening purposes CT60 cells (650 cells/well) or CT43 cells (700 cells/well) in 301 of growth medium A with 10% FBS were seeded in Costar 384 well black polystyrene flat, clear bottomed tissue culture treated plates (Corning, Inc., NY) to obtain ˜80% confluency when cells were analyzed.

Normal human fibroblasts (GM5659E) were grown in MEM with 1% P/S and 10% FBS. For microscopy, fibroblasts were plated in normal growth medium on glass-bottomed 35 mm dishes or in 384-well plates.

Compound addition: The library compounds were formatted for screening in the Rockefeller University High Throughput Screening Facility. Cells were treated with the compounds from the chemical library one day after plating. We added 0.11 of each compound (5 mM stock in DMSO) to 25 μl of screening Medium S composed of medium A supplemented with 1% FBS and 20 mM 4-(2-hydroxyethyl)-1-pipiperazine ethane sulphonic acid (HEPES) in Falcon 384-well V-bottomed polypropylene plates using a Packard MiniTrak™ robotic liquid handling system. To obtain ˜10 μM final concentration, 23 μl of the premixed compounds were dispensed into the plates containing cells and 301 of culture medium A. For primary screening, 352 test compounds were added to each plate, and the remaining 32 wells were used as a control with only DMSO added. All plates were incubated with compounds for 16 h at 37° C. Plates were then washed three times with phosphate buffered saline pH 7.4 (PBS) using a Bio-Tek Elx405 plate washer (Bio-Tek Instruments Inc., Winooski, Vt.). For each wash cycle, 701 of PBS was dispensed followed by aspiration with a residual volume of 16 μl per well. Finally, cells were fixed with 1.5% PFA in PBS for 20 min at room temperature, followed by 3 more washes with PBS.

Fluorescence labeling. To the fixed cells, filipin was added at a final concentration of 50 μg/ml in PBS for 45 m at room temperature to label free cholesterol. Cells were finally washed three times with PBS, and images were acquired immediately after labeling.

Fluorescence Microscopy: A Discovery-1 automatic fluorescence microscope from Molecular Devices Corporation equipped with a Xenon-arc lamp (PerkinElmer, Calif.), Nikon 10× Plan Fluor 0.3NA objective, and Photometrics CoolSnapHQ camera (1392×1040 pixels; Roper Scientific, Tucson, Ariz.) was used to acquire images. Filipin images were acquired using 360/40 nm excitation and 480/40 nm emission filters with a 365 DCLP (DiChroic Long Pass) filter. The image files were stored on the local host computer before being transferred to a server.

Plates were transported from plate hotels using a CRS CataLyst Express robot (Thermo Electron Corp). Images were acquired at two sites per well, each approximately 50 μm from the center of the well with 75 ms exposure time per image using 2×2 binning. Automatic focusing was carried out by different methods for the primary and secondary screens. In the primary screen, each well was focused over a ±150 μm range and each site per well was focused over a ±20 μm range using image-based focusing and the MetaMorph auto-focusing algorithm. Images for focusing were acquired with 15 ms exposure time using 8×8 binning to reduce photo-bleaching. For the secondary screen, laser-based auto-focusing (LAF v.2 from Molecular Devices) was used to find the bottom of the plate. Image-based focusing was used to determine the offset between the bottom of the plate and the cells, and then each site was refocused over a 20 μm range. Acquisition time per plate was 60-75 min regardless of the focusing method. 696×520 pixel images were acquired at 12 intensity bits per pixel. Each pixel is 1.25×1.25 μm in the object.

Image analysis: Images of filipin-stained cells were analyzed using Metamorph Discovery-1 image analysis software. Two different image analysis assays were developed: (1) Average filipin intensity assay and (2) LSO compartment ratio assay. First, to correct for shading an image was created by averaging all of the images from a plate and smoothing the averaged image using a low pass filter. Then each pixel in an image was multiplied by the average intensity of the shading image, and the resulting pixel intensities were divided by the shading image on a pixel-by-pixel basis. Background was subtracted from each shading-corrected image by determining the 5th percentile intensity value of the image and subtracting this value from each pixel in the image. At the plating density used, all fields had at least 5% of the imaged areas that was cell-free. Next, two different thresholds were applied to the filipin images. For the first, a low threshold was set to include all areas occupied by cells. The outlines of cells using the selected values were comparable to cell outlines in transmitted light images. A second, higher threshold was set for brightly stained regions in CT60 and/or CT43 cells by selecting bright areas of filipin staining, with the intention of mainly identifying the LSOs in the perinuclear region of the cells. For the Average filipin intensity assay, using the low threshold alone, we measured total filipin intensity above the low threshold divided by the number of pixels above the lower threshold for each field. This gave an average filipin intensity per cell area. For the LSO compartment ratio we measured the total filipin intensity selectively in the region above the higher threshold divided by the number of pixels in the lower threshold. This gave a measure of the total intensity of LSO filipin per cell area.

$$Average filipin intensity = \frac{\begin{matrix}Total intensity above \\ low threshold\end{matrix}}{\begin{matrix}Number of pixels above low \\ threshold\end{matrix}}$$

$$LSO Compartment Ratio = \frac{\begin{matrix}Total intensity above \\ high threshold\end{matrix}}{\begin{matrix}Number of pixels above low \\ threshold\end{matrix}}$$

Normalized values were obtained by dividing the values in the presence of each compound by the values obtained in presence of solvent control for each plate. Similar methods were used to analyze the effects of compounds on human fibroblasts treated with U18666A to induce cholesterol retention in LSOs.

Immunofluorescence: CT60 cells were grown to 70% confluency on glass coverslip bottom dishes. After 24 h, compounds were added (10 μM) to the cells in screening medium supplemented with 20 mM HEPES. Following 16-22 h compound treatment, cells were washed three times with PBS and fixed with 3.3% PFA for 20 min at room temperature. Cells were than treated with 50 mM ammonium chloride for 10 min and washed three times with PBS. After blocking with 0.1% BSA for 20 min, cells were washed with PBS, permeabilized with 0.05% saponin and incubated with anti-LBPA (1:100) (gift from J. Gruenberg, Univ. of Geneva) for 30 min at RT. Cells were washed three times with PBS and incubated with goat anti-mouse IgG-Alexa546 (1:200) and 100 μg/ml filipin for 30 min. Finally, cells were washed three times with PBS, and images were acquired on a Leica DMIRB microscope (Leica Mikroscopie und Systeme GmbH, Germany) equipped with a Princeton Instruments (Princeton, N.J.) cooled CCD camera driven by MetaMorph Imaging System software. All images were acquired using an oil immersion objective (25×, 1.4 NA). Alexa 546-Tf was imaged using a standard rhodamine filter cube, and filipin was imaged using a Leica A4 cube [360 nm (40 nm band pass) excitation filter and 470 nm (40 nm band pass) emission filter]. Images were analyzed using Metamorph Discovery-1 image analysis software to estimate cholesterol and LBPA content in the presence and absence of the compounds using the image analysis algorithms as for screening.

### Example 2

### Dose Dependence Assay

Dose Dependence: Using the same methods as for the screening, compounds were tested at 10 μM, 3.33 μM, 1.11 μM, 370 nM and 123 nM in four wells each. Batches of selected compounds were purchased from Chemical Diversity, and 10 mM stocks in DMSO were made. A secondary stock plate of 2× concentration (20 μM, 6.66 μM, 2.22 μM, 740 nM and 246 nM) of compounds was prepared in screening medium S. To obtain the final concentrations, 301 of this secondary stock was added to the cells in each well containing 30 μl of growth medium A supplemented with 10% FBS. The final DMSO concentration (0.2% v/v) was the same in all wells. Cells were plated at 650 cells per well in 30 μl of growth medium A supplemented with 10% FBS in Costar 384 well plates. After 20 h of incubation in the presence of the compounds, cells were washed with PBS, fixed with PFA and stained with filipin as described for the screening assay. Dose dependence determination of the initial 14 hit compounds from the primary library was done at least 5 times in CT60 cells and 3 times in CT43 cells in separate experiments. Dose dependence determination of the 7 hit compounds from the secondary library was carried out at least 3 times in CT60 cells and 2 times in CT43 cells in separate experiments.

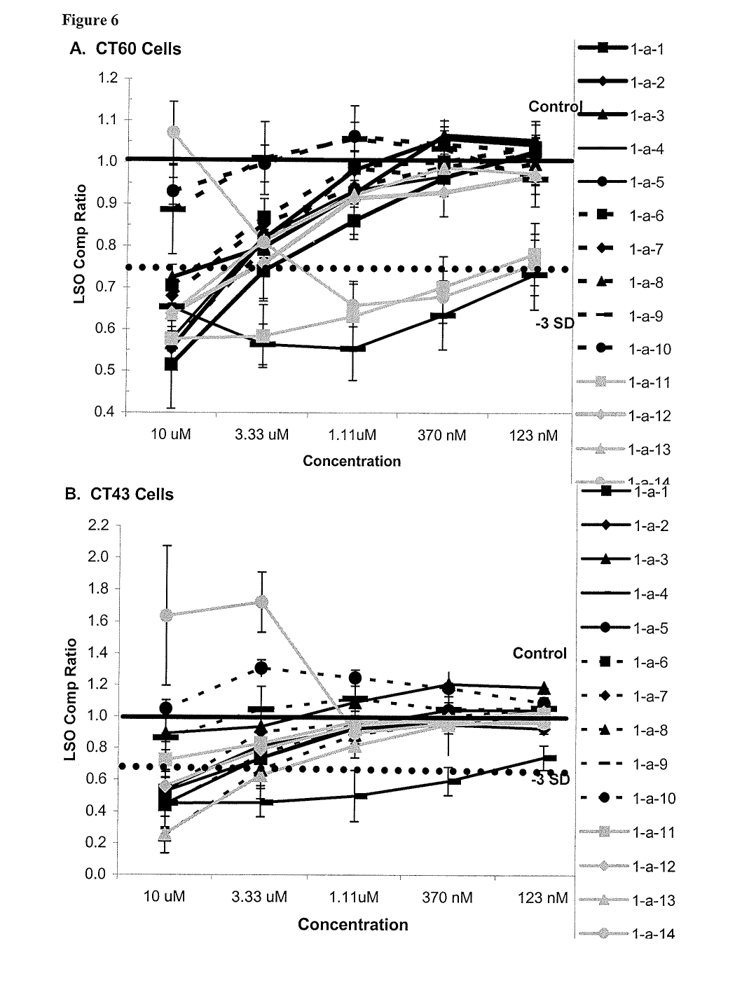

The dose-response curves for the 14 selected compounds in an assay identical to the screening assay are shown in FIGS. 6A-B. Three of the compounds reduced filipin staining 3 SD below the solvent-treated mean at 1.1 μM in CT60 cells, and cells treated with these same compounds were more than 2 SD below the solvent-treated mean at 123 nM (FIG. 6A). Compound 1-a-14 had an unusual dose-response curve, which is may be associated with its cytotoxicity at higher concentrations. To determine whether the effects were specific to the CT60 cell line, we also tested the dose response of the 14 selected compounds on CT43 cells, another NPC1 mutant cell line derived from the parental cell line, 25RA. Most of the compounds were effective at more than 3 SD below the untreated mean at 10 μM but lost their effect at lower concentrations on CT43 cells (FIG. 6B). It is interesting to note that the general trends of the effects of these 14 compounds are similar in both cell lines, although the compounds generally are more effective at a given dose in the CT60 cells.

### Example 3

### Time Course Assay

The effects of the compounds were determined at 1.11, 3.33 and 10 μM concentrations after 4, 20 and 48 h using methods similar to the dose dependence. The CT60 cells were seeded in three 384 well plates at 600 cells/well in growth medium on day 1. To maintain the same density of cells at the final time point, compounds were added chronologically. After overnight incubation, in the first plate (for 48 h time point) compounds diluted in medium S were added in wells to achieve the final concentrations of 1.11, 3.33 and 10 μM. In the second plate compounds were added in similar fashion 52 h after seeding the cells and allowed to incubate for 20 h. Finally, in the third plate compounds were added 68 h after seeding the cells and allowed to incubate for 4 h. All three plates were washed with PBS three times, fixed with 1.5% PFA and stained with 50 μg/ml filipin. Measurements were made from 4 wells for each condition in each experiment, and the experiments were repeated three times each for CT60 and CT43 cells (CT43 data not shown). Images were acquired at 10× magnification on Discovery-1 automatic fluorescence microscope for 2 sites/well and analyzed to obtain the LSO compartment ratio.

### Example 4

### Toxicity Assay

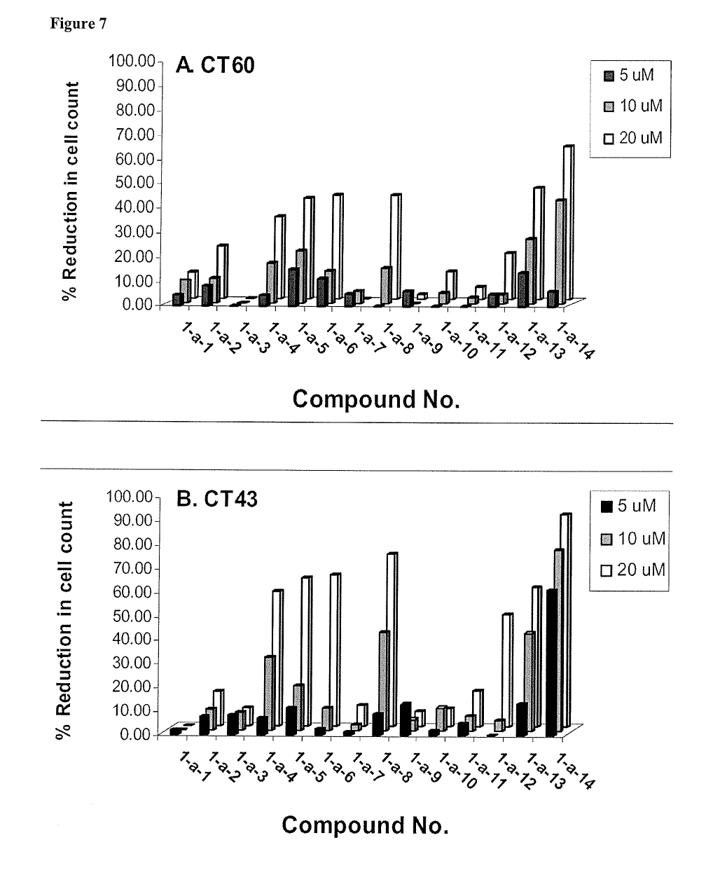

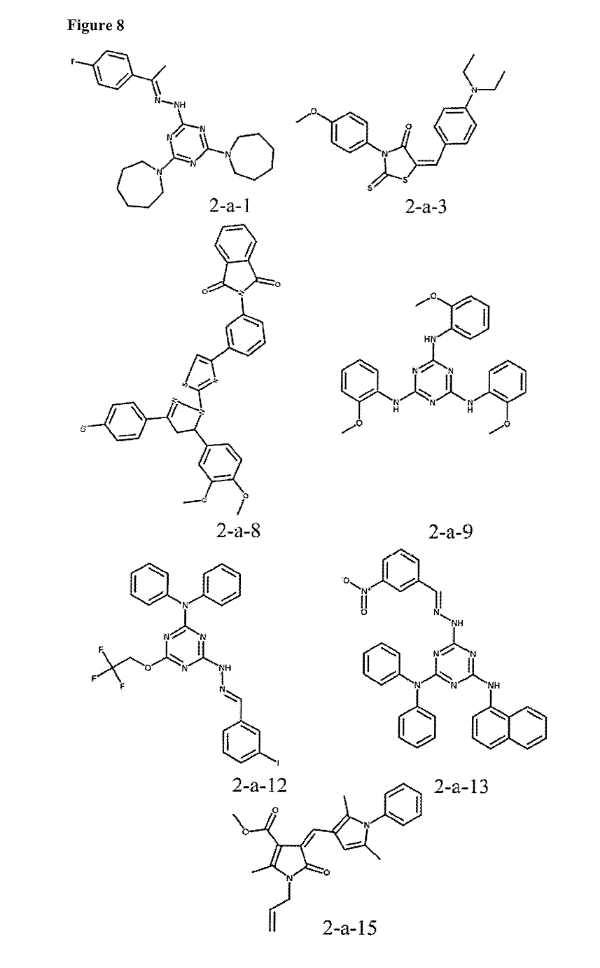

To measure toxicity, we treated CT60 and CT43 cells with compounds at 5, 10 and 20 μM for 24 h. The cell number per well was compared to control cells treated with DMSO (FIG. 7). Many of the compounds did not cause a significant decrease in cell number at 10 μM after 24 h incubation. Compound 1-a-14 did cause a 50% reduction in cell number at 10 μM for CT60 and an 80% reduction for CT43 cells. Compounds 1-a-4, 5, 6, 8 and 13 were partially toxic as indicated by 20-30% reduction in cell number after 24 h at 10 μM.

Procedure Using Cell Count Assay: Compounds were added to CT60 and CT43 cells plated in 384 well plates at 0 (DMSO solvent control), 5, 10 and 20 μM concentrations in quadruplicate using methods similar to the dose dependence assay, except that the cells were stained with Hoechst 33258 nuclear stain. The final DMSO concentration in each well was 0.2%. For the control cells an equivalent amount of DMSO was added to the cells. The cells were incubated for 24, 48 and 72 h. After each time period, cells were washed and then fixed with 1.5% PFA. After washing the cells three times with PBS, the nuclei of the cells were stained using 5 μg/ml Hoechst 33258 (25 mg/ml stock solution in DMSO) in PBS for 45 m at room temperature. Finally, cells were washed three times with PBS, and images were obtained using a Nikon 4× Plan Apo 0.2NA objective. For Hoechst imaging we used the same filter set as for filipin. We collected one 520×696 pixel image per well at 12 intensity bits per pixel. Each pixel is 3.125 by 3.125 μm in 13 the object. Cells were counted by defining the standard area of single nuclei (˜200 μm²) interactively for each plate using the Integrated Morphometry Analysis function of MetaMorph. The number of standard areas per object above a threshold was determined, and the total number of standard areas per image was used as the cell count. The percent reduction in the number of cells compared to DMSO control was calculated for each concentration and time.

### Example 5

### Gas Chromatography Cholesterol Assay

The cholesterol lowering effect of the 14 hit compounds identified by filipin labeling was measured by an alternative chemical method that involved GC separation of solvent-extracted cellular lipids (43). CT60 cells were treated with compounds at 10 μM under the same conditions as in the screening assays. Since the GC assay estimates FC content per cell, the values are compared to the average filipin intensity assay (Table 1). As indicated in Table 1, most of the 14 hit compounds induce a relatively modest decrease of overall FC content of the cells, as measured by the average filipin assay. The hits from the first screen had variable effects on cellular cholesterol; including some compounds that apparently increased free cholesterol in the cells.

Table 1. Free cholesterol content by average filipin intensity and GC assays for CT60 cells treated with 14 hit compounds from primary library.

| | Average filipin intensity | μg FC¹/μg Protein by GC |
|---|---|---|
| Compound No. | Fraction of control | Fraction of control |
| 1-a-1 | 0.87 ± 0.04 | 1.02 ± 0.03 |
| 1-a-2 | 0.88 ± 0.04 | 1.15 ± 0.05 |
| 1-a-3 | 0.95 ± 0.04 | 1.27 ± 0.07 |
| 1-a-4 | 1.01 ± 0.04 | 0.87 ± 0.07 |
| 1-a-5 | 0.89 ± 0.04 | 1.18 ± 0.06 |
| 1-a-6 | 0.92 ± 0.04 | 1.37 ± 0.01 |
| 1-a-7 | 0.89 ± 0.04 | 1.04 ± 0.13 |
| 1-a-8 | 0.90 ± 0.04 | 1.31 ± 0.10 |
| 1-a-9 | 0.99 ± 0.04 | 0.86 ± 0.07 |
| 1-a-10 | 0.96 ± 0.04 | 1.23 ± 0.08 |
| 1-a-11 | 0.90 ± 0.04 | 0.82 ± 0.07 |
| 1-a-12 | 0.91 ± 0.04 | 0.73 ± 0.03 |
| 1-a-13 | 0.97 ± 0.04 | 1.54 ± 0.11 |
| 1-a-14 | 1.24 ± 0.04 | 1.44 ± 0.16 |

¹FC—Free cholesterol

CT60 or CT43 cells were plated on day 1 in 6-well plates. Hit compounds were added to the cells at 10 μM concentration. Cells were incubated for 24 h. Cellular lipids were extracted with hexane: iso-propyl alcohol (3:2 v/v). The lipid extracts were dried and re-suspended in hexane followed by separation on gas chromatograph using β-sitosterol as an internal standard.

Procedure: CT60 cells were plated in 6-well plates in Ham's F-12/1.176 g/L sodium bicarbonate/2 g/L glucose/10% FBS. Following 24 h incubation, cells were treated with 10 μM of each compounds, medium being replaced with Ham's F-12/1.176 g/L sodium bicarbonate/2 g/L glucose/5.5% FBS/20 mM HEPES. The change of the medium in the wells was performed to have experimental conditions similar to those used for the screening once chemical compounds were added to the cells. After 18 h treatment with compounds, cells were washed twice with Hank's balanced salt solutions (HBSS). Cellular lipids were extracted with hexane/isopropyl alcohol (3:2 v/v) (43) dried and resuspended in hexane followed by separation on a gas chromatograph (GC) using following conditions. A Hewlett Packard GC model HP 5890 series II (Palo Alto, Calif.) equipped with a flame ionization detector, split-splitless injector, and 15 m×0.53 mm HP-5 capillary column coated with 1.5 μM film thickness of 5% phenyl methyl siloxane was used to separate free cholesterol. The injection temperature was maintained at 255° C. and oven temperature was held isothermally at 260° C. using helium as a mobile phase at 30 mL/min flow rate. The free cholesterol (FC) was quantified using beta-sitosterol as internal standard to correct for lipid losses during extraction.

### Example 6

### Screening of Second Library

A second library containing 3962 compounds was evaluated. These compounds were selected based upon the chemical similarity in terms of the Tanimoto coefficient (47).

We carried out a screen of these 3962 compounds using a protocol similar to the primary library screen except that compounds were initially screened at two concentrations, 10 μM and 1 μM. Each compound was added in a single well, and images were acquired for two positions per well and averaged to give a single value. Two full screens of the secondary library were carried out at both concentrations. The images were analyzed using both the average filipin intensity and the LSO compartment ratio methods. At 10 μM, we found 574 compounds that were 3 SD below controls as determined by one of the analysis methods and 34 compounds that were 3 SD below controls by both of the analysis methods. At 1 μM, we found 202 compounds that were 3 SD below controls by at least one of the analysis methods, and 6 compounds that were 3 SD below controls by both methods.

We cherry-picked these 202 compounds and re-screened them twice at 1 μM, 300 nM and 100 nM. We added each compound to four different wells at each concentration, and measurements from images at two positions per well were averaged. With the two screens, we obtained a total of 8 values by each analysis method for every compound at 1 μM, 300 nM and 100 nM. All together, we had a total of 10 values (2 from the first screen and 8 from the cherry picking round) for 1 μM and 8 values for 300 nM and 100 nM for each analysis method (average filipin intensity and LSO compartment ratio). Based on these data, we selected 7 compounds for further analysis, and their chemical structures are shown in FIG. 8.

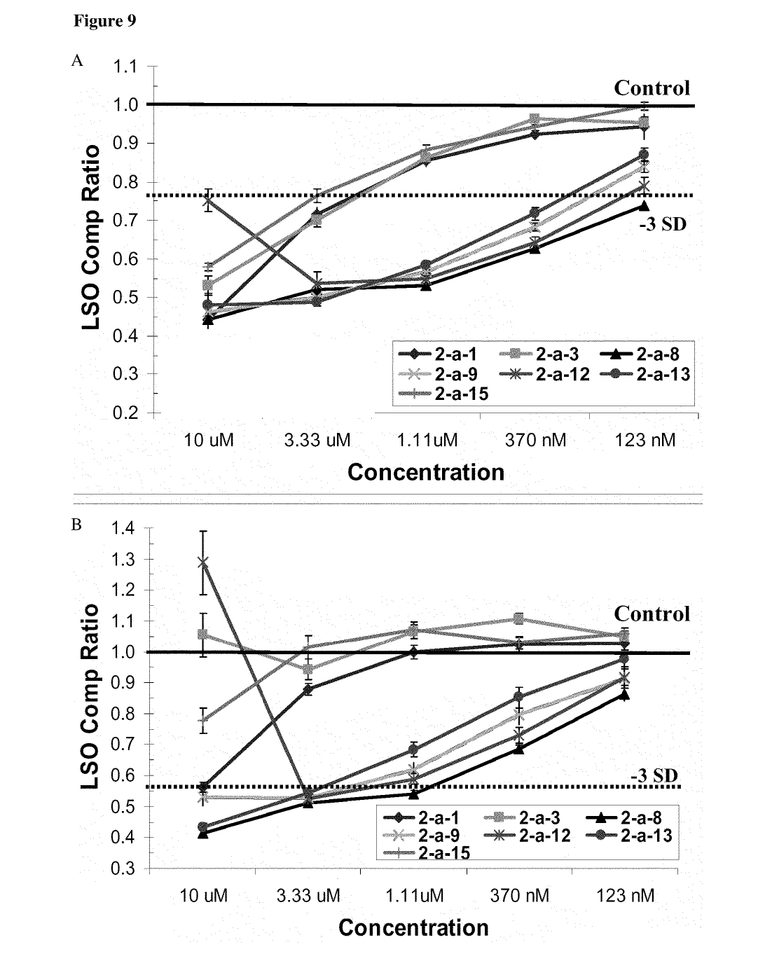

The dose response curves for the 7 selected compounds are shown in FIG. 9. The data indicate that four compounds (2-a-8, 2-a-9, 2-a-12 and 2-a-13) showed more than a 3 SD reduction in the LSO compartment assay at 370 nM, and three compounds (2-a-8, 2-a-9, and 2-a-12) also showed an effect 2 SD below solvent control at 123 nM on CT60 cells. As with the hits from the first round, most of these compounds were also effective on CT43 cells (FIG. 9B).

### Example 7

### LDL Uptake

CT60 cells were grown to 70% confluency in 96 well special optics plates (Corning, Inc., Corning, N.Y.). After 24 hours, cells were incubated with DiI-LDL (6 μg/ml) and hit compounds (10 μM) in screening medium supplemented with 20 mM HEPES. Each compound was added to 8 wells and an equivalent amount of DMSO with DiI-LDL was added to control wells. After 20 h, cells were washed three times with PBS, fixed with 1.5% PFA for 20 min, and stained with 50 μg/ml filipin for 45 min. Images were acquired using the Discovery-1 automatic fluorescence microscope at 20× magnification. DiI-LDL images were acquired using 535 nm/40 nm excitation filter and 610 nm/60 nm band pass with a Chroma 51001bs DiChroic filter. Filipin images were acquired as described above. Images were acquired for 4 sites per well, yielding 32 images per compound. The DiI-LDL images were background and shade corrected as described above. A low threshold was set to define the cell area based on filipin images. Finally, the average DiI-LDL intensity was measured per cell area.

As shown in Table 2, all of the hit compounds from the secondary library, except 2-a-15, caused a decrease in LDL uptake during a 20 hour incubation. It will require further work to determine if this is a primary effect of some of the compounds or if the decrease in LDL uptake is secondary to the release of cholesterol from the LSOs. Although CT60 cells have a partial defect in SCAP function as a consequence of a point mutation in one SCAP allele, it would be expected that the cells would respond to increased cholesterol by decreasing expression of LDL receptors.

Table 2. DiI-LDL uptake by CT60 cells treated 7 hit compounds from secondary library.

| | DiI Intensity/cell area |
|---|---|
| Compound No. | Fraction of control |
| 2-a-1 | 0.43 ± 0.01 |
| 2-a-3 | 0.62 ± 0.01 |
| 2-a-8 | 0.77 ± 0.01 |
| 2-a-9 | 0.85 ± 0.01 |
| 2-a-12 | 0.72 ± 0.01 |
| 2-a-13 | 0.72 ± 0.01 |
| 2-a-15 | 1.00 ± 0.01 |

Cells were incubated with DiI-LDL (6 μg/ml) in the presence of compounds (10 μM) for 20 hours. The intensity per unit cell area was measured as described in Methods. Values are normalized to the intensity per cell area for control (solvent-treated) cells. Values are based on averages from 32 images from 8 wells for each condition. ±SEM.

### Example 8

### Toxicity Assay for Compounds from Second Library

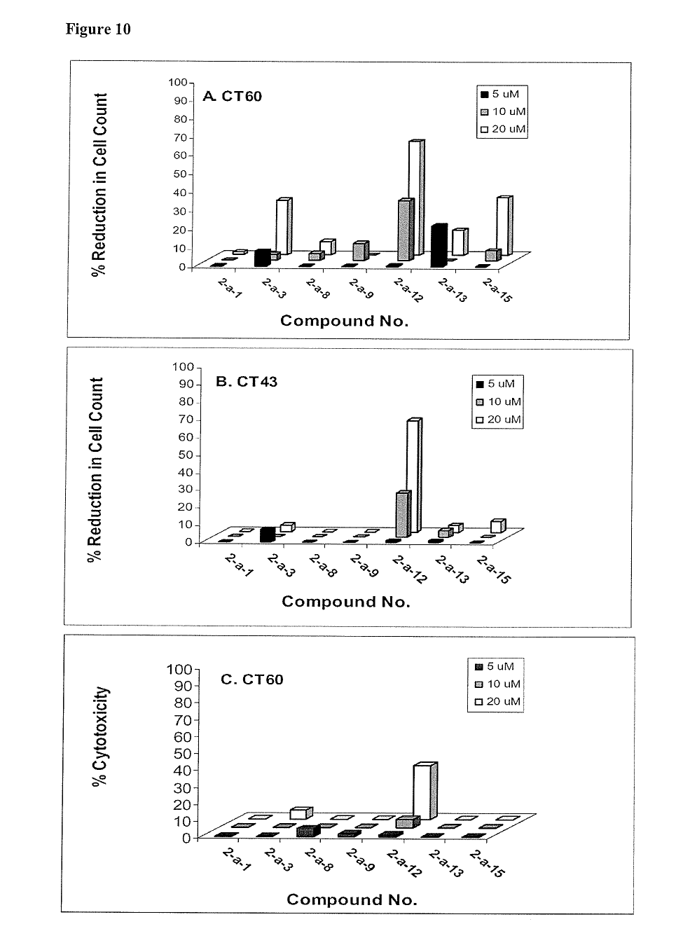

FIG. 10 shows the results of a toxicity assay on the 7 hit compounds from the second library. Compound 2-a-12 caused a 75% reduction in the number of cells after 24 h at 20 μM for both CT60 and CT43 cells. The other compounds caused either no loss of cells or only a slight loss under the conditions where they reduced the filipin staining. Cytotoxicity of these compounds was assessed measurement of LDH release into the medium. As shown in FIG. 10 C, the cytotoxicity measured by this assay was less than the reduction in cell count, indicating that the compounds may have slowed cell growth without causing cell death after 24 hours.

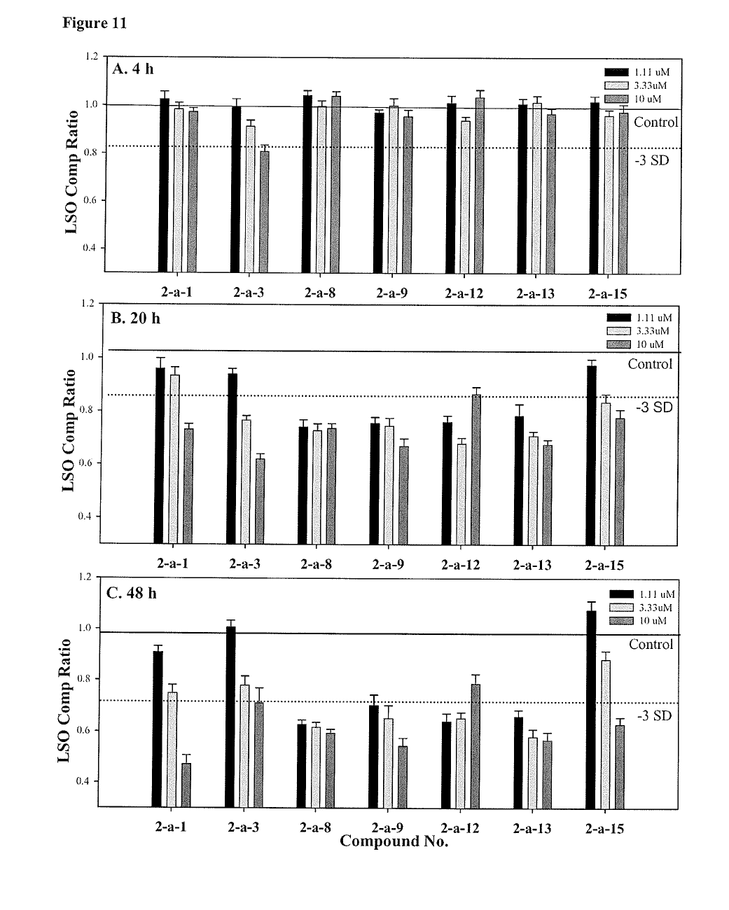

The time course of the reduction in the LSO compartment ratio at 1.1, 3.3 and 10 μM is shown in FIG. 11. After a 4 hr treatment, compound 2-a-3 showed a more than 3 SD decrease in the LSO compartment ratio. After 20 hr, all the compounds showed a reduction in the LSO compartment ratio at 10 μM, and several compounds were effective at 1.11 and 3.33 μM. After the 48 hr treatment, the effect in reducing LSO compartment ratio was generally retained. Six of the seven compounds selected in the secondary screen had low cytotoxicity and reduced FC significantly not only in LSOs but also in the whole cell. Several of these compounds were effective at concentrations below 0.5 μM. Table 3 summarizes the effect of these 6 hit compounds from the secondary library in terms of cholesterol reduction as well as toxic effects.

Table 3. Summary of effects of seven compounds from secondary library on CT60 cells

| | | | Average | μg FC¹/μg | Protein | Cell Count |
|---|---|---|---|---|---|---|
| | Lowest | | Filipin Assay | Protein (GC) | Content | After 24 h |
| | Effective Dose | | @ 10 μM dose | @ 10 μM dose | (μg/well) | @ 10 μM dose |
| Compound | (LSO Assay) | | Fraction of | Fraction of | Fraction of | Fraction of |
| No. | (−3 SD @ 20 h) | | Control | Control | Control | Control |
| 2-a-1 | 3.33 | μM | 0.74 ± 0.02 | 0.65 ± 0.02 | 0.94 ± 0.03 | 1.00 |
| 2-a-3 | 3.33 | μM | 0.75 ± 0.01 | 0.81 ± 0.03 | 0.92 ± 0.04 | 1.00 |
| 2-a-8 | 123 | μM | 0.74 ± 0.01 | 0.76 ± 0.03 | 0.94 ± 0.04 | 0.96 |
| 2-a-9 | 370 | μM | 0.74 ± 0.01 | 0.75 ± 0.04 | 0.94 ± 0.05 | 0.90 |
| 2-a-12 | 370 | μM | 0.87 ± 0.01 | — | — | 0.67 |
| 2-a-13 | 370 | μM | 0.75 ± 0.02 | 0.79 ± 0.04 | 0.97 ± 0.03 | 1.00 |
| 2-a-15 | 3.33 | μM | 0.79 ± 0.01 | 0.89 ± 0.03 | 0.84 ± 0.02 | 0.94 |

¹FC—Free cholesterol

### Example 9

### LDH Cytotoxicity Assay

Cytotoxicity of hit compounds was measured by an LDH release assay kit according to the manufacturer's instructions (Roche Diagnostic GmbH, Penzberg, Germany). CT60 cells were plated in 96-well plates (Costar, Corning Inc., Corning, N.Y.) at a density of 3500 cells/well and incubated for 24 h. Compounds were added to the CT60 cells at 0 (DMSO solvent control), 5, 10 and 20 μM concentrations in triplicate using methods similar to the dose dependence assay. After 24 h treatment, 1001 of tissue culture supernatant was removed, and LDH activity was determined by measuring absorbance at 492 nm using a SpectraMax M2 fluorescence plate reader (Molecular Devices Inc., Sunnyvale, Calif.). The experiment was repeated three times, thus an average of nine data points is reported.

As shown in FIG. 10C, the cytotoxicity measured by this assay was less than the reduction in cell count, indicating that the compounds may have slowed cell growth without causing cell death after 24 hours.

### Example 10

### Assay on Normal Human Fibroblasts

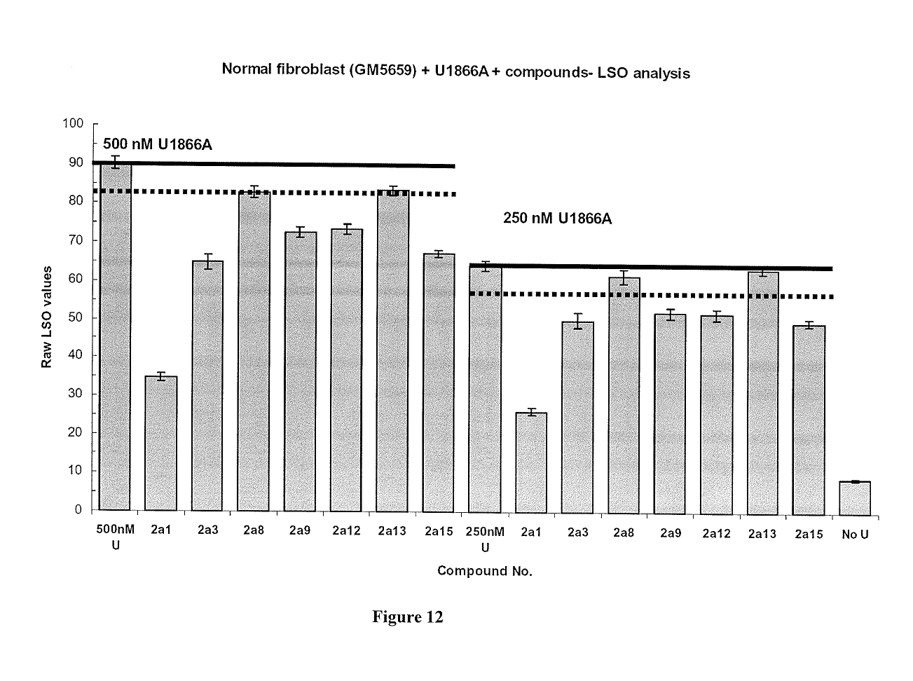

All of the results described above were obtained with CHO cells that have a mutation in SCAP in addition to their mutations in NPC1. In order to determine whether the compounds might have effects on other types of cells, we treated normal human fibroblasts with compound U18666A, which induces cholesterol accumulation similar to that seen in NPC cells (N L Jacobs et al., J Lipid Res. 1997 October; 38(10):1973-87). We found that treatment of cells with 250 nM or 500 nM U18666A for 20 hours caused a significant increase in cholesterol accumulation as seen by filipin staining (data not shown) and as measured by the LSO compartment ratio method (FIG. 12). When cells were treated with hit compounds from the secondary library, several of these compounds caused a significant decrease in the filipin staining (FIG. 12). In particular, compound 2-a-1 caused a dramatic decrease in filipin staining.

### Example 11

### Assay on 25RA CHO Cells

25RA CHO cells were incubated with 10 μM concentrations of compound 2-a-1, 2-a-3,2-a-8, 2-a-9, and 2-a-13. Cells were loaded with ³H cholesterol and then chased overnight in the presence or absence of the various compounds. The media over the cells were removed, and radioactivity released from the cells was measured. The cells were then solubilized, and radioactivity remaining in the cells was measured. The data (FIG. 13) indicates that these compounds promote efflux of cholesterol from 25-RA cells, a CHO cell line without a Niemann-Pick defect that was the parental cell line used to select the CT43 and CT60 cells used in our screens above. The 25-RA cells do have a defect in SCAP, which alters cholesterol homeostasis, including increased synthesis of cholesterol.

### Examples 12

### Inhibition of Lysosomal Acid Lipase

Certain compounds disclosed herein may have their therapeutic effect by inhibiting lysosomal acid lipase (LAL). In particular, compounds 1-a-4,1-a-11, 1-a-14, 2-a-3, 2-a-8, 2-a-9, 2-a-13, 2-a-15, and other compounds within the scopes of the generic structures that define the named compounds may inhibit hydrolysis of cholesteryl esters by LAL.

1. A method of treating a patient suffering from a disorder characterized by cellular accumulation of cholesterol, comprising the step of:
administering to a patient in need thereof a therapeutically effective amount of a compound of formula III, wherein formula III is represented by: 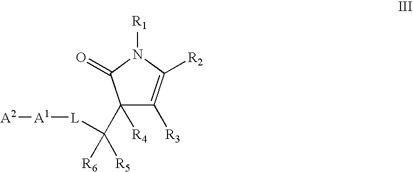
wherein,
R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);
R² is cycloalkyl, heterocycloalkyl, cycloalkenyl, heterocycloalkenyl, aryl, heteroaryl, aralkyl, heteroaralkyl, or alkyl;
R³ is hydrogen, alkyl, —CO₂R⁸, or —C(O)N(R⁷)(R⁸);
R⁴ and R⁵ represent independently H or alkyl; or R⁴ and R⁵ taken together form a bond;
R⁶ and R⁷ represent independently for each occurrence H or alkyl;
R⁸ represents independently for each occurrence alkyl, cycloalkyl, aryl, heteroaryl, aralkyl, or heteroaralkyl;
L is a bond, —C(R⁷)₂—, or —(CR⁷═CR⁷);
A1 and A2 represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl-;
and n is 1, 2, 3, 4, 5 or 6.

2. The method of claim 1, wherein the disorder is Niemann-Pick disease type C.

3. The method of claim 1, wherein the disorder is atherosclerosis.

4. The method of claim 1, wherein the disorder is a Lysosomal storage disorder arising from a defect in sphingolipid or glycosphingolipid metabolism.

5-11. (canceled)

12. The method of claim 1, wherein R¹, R², A¹, and A² represent independently aryl or heteroaryl; R³ is hydrogen or alkyl; R⁶ is H or alkyl; and L is a bond.

13. The method of claim 1, wherein R¹ is —(C(R⁷)₂)ₙ—(CR⁷═C(R⁷)₂); R² is alkyl; R³ is alkyl, —CO₂R⁸, or —C(O)N(R⁷)(R⁸); R⁴ and R⁵ represent independently H or alkyl; or R⁴ and R⁵ taken together form a bond;
R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

14-27. (canceled)

28. The method of claim 1, wherein said compound is

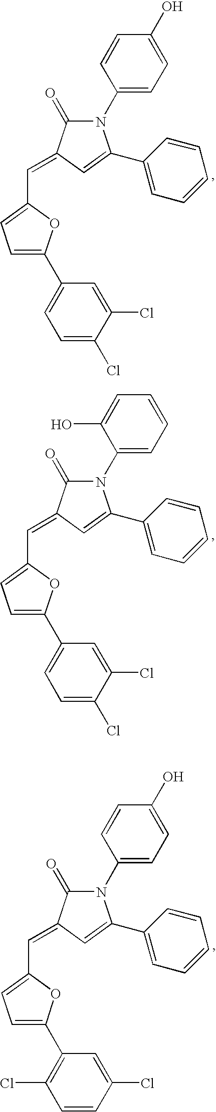

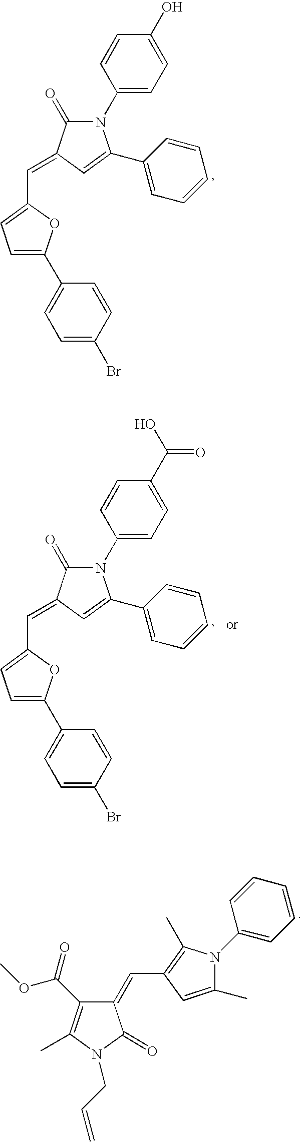

29. A method of reducing the amount of cholesterol in a cell, comprising the step of:
exposing a mammalian cell to a compound of formula III, wherein formula III is represented by: 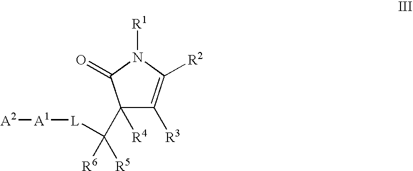
wherein,
R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);
R² is cycloalkyl, heterocycloalkyl, cycloalkenyl, heterocycloalkenyl, aryl, heteroaryl, aralkyl, heteroaralkyl, or alkyl;
R³ is hydrogen, alkyl, —CO₂R⁸, or —C(O)N(R⁷)(R⁸);
R⁴ and R⁵ represent independently H or alkyl; or R⁴ and R⁵ taken together form a bond;
R⁶ and R⁷ represent independently for each occurrence H or alkyl;
R⁸ represents independently for each occurrence alkyl, cycloalkyl, aryl, heteroaryl, aralkyl, or heteroaralkyl;
L is a bond, —C(R⁷)₂—, or —(CR⁷═CR⁷)—;
A¹ and A represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;
and n is 1, 2, 3, 4, 5 or 6.

30. The method of claim 29, wherein said compound reduces the amount of cholesterol in said cell by increasing cholesterol efflux from said cell.

31. The method of claim 29, wherein said compound reduces the amount of cholesterol in said cell by inhibiting cholesterol uptake by said cell.

32. The method of claim 29, wherein said compound reduces the amount of cholesterol by inhibiting cholesterol synthesis by said cell.

33. The method of claim 29, wherein said compound reduces the amount of cholesterol in said cell by promoting esterification of cholesterol in said cell.

34. The method of claim 29, wherein said cell is a human cell.

35. The method of claim 29, wherein said cell has a Niemann-Pick Type C defect.

36-42. (canceled)

43. The method of claim 29, wherein R¹, R², A¹, and A² represent independently aryl or heteroaryl; R³ is hydrogen or alkyl; R⁶ is H or alkyl; and L is a bond.

44. The method of claim 29, wherein R¹ is —(C(R⁷)₂)ₙ—(CR⁷═C(R⁷)₂); R² is alkyl; R³ is alkyl, —CO₂R⁸, or —C(O)N(R⁷)(R⁸); R⁴ and R⁵ represent independently H or alkyl; or R⁴ and R⁵ taken together form a bond; R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

45-58. (canceled)

59. The method of claim 29, wherein said compound is

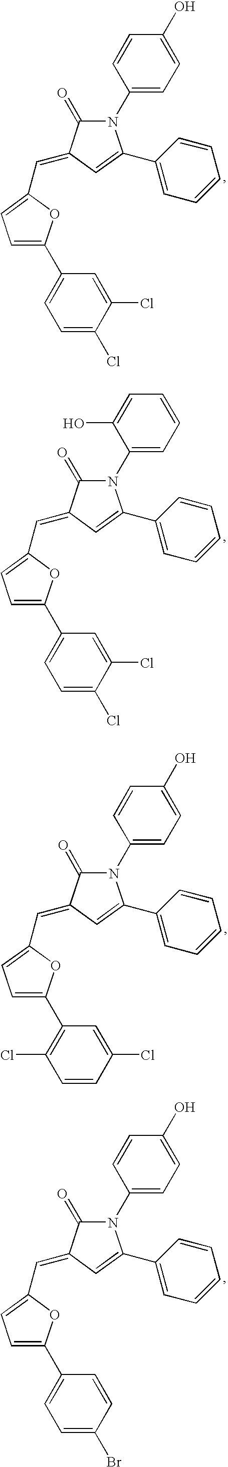

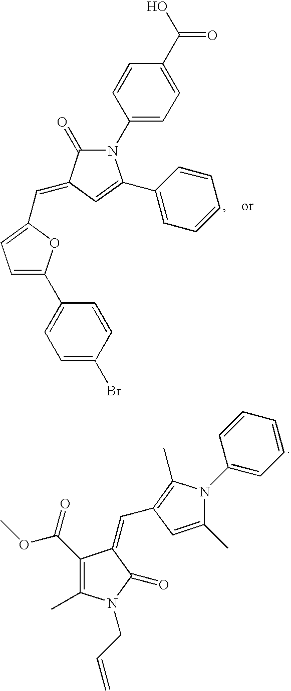

60-75. (canceled)

76. A compound represented by formula XVIII: 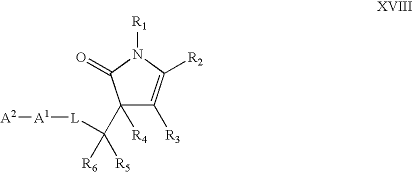
wherein,
R¹ is cycloalkyl, heterocycloalkyl, cycloalkenyl, heterocycloalkenyl, aryl, heteroaryl, aralkyl, heteroaralkyl;
R² is cycloalkyl, heterocycloalkyl, cycloalkenyl, heterocycloalkenyl, heteroaryl, aralkyl, heteroaralkyl, or alkyl;
R³ is hydrogen, alkyl, —CO₂R⁸, or —C(O)N(R⁷)(R⁸);
R⁴ and R⁵ represent independently H or alkyl; or R⁴ and R⁵ taken together form a bond;
R⁶ and R⁷ represent independently for each occurrence H or alkyl;
R⁸ represents independently for each occurrence alkyl, cycloalkyl, aryl, heteroaryl, aralkyl, or heteroaralkyl;
L is a bond, —C(R⁷)₂—, or —(CR⁷═CR⁷)—; and
A¹ and A² represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;
or a compound represented by formula XIX: 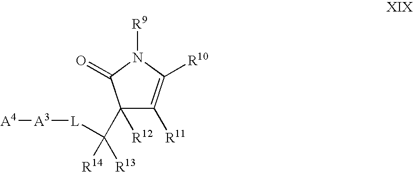
wherein,
R⁹ is cycloalkyl, heterocycloalkyl, cycloalkenyl, heterocycloalkenyl, heteroaryl, aralkyl, heteroaralkyl, or —(C(R¹⁵)₂)ₙ—(CR¹⁵═C(R¹⁵)₂);
R¹⁰ is aryl;
R¹¹ is hydrogen, alkyl, —CO₂R⁶, or —C(O)N(R¹⁵)(R¹⁶);
R¹² and R¹³ represent independently H or alkyl; or R¹² and R¹³ taken together form a bond;
R¹⁴ and R¹⁵ represent independently for each occurrence H or alkyl;
R¹⁶ represents independently for each occurrence alkyl, cycloalkyl, aryl, heteroaryl, aralkyl, or heteroaralkyl;
L is a bond, —C(R¹⁵)₂—, or —(CR¹⁵═CR¹⁵)—;
A³ represents a bivalent cycloalkenyl, heterocycloalkenyl, aryl, heteroaryl, aralkyl, heteroaralkyl, —(CR¹⁵═CR¹⁵)-aryl-, or —(CR¹⁵═CR¹⁵)-heteroaryl-;
A⁴ represents cycloalkenyl, heterocycloalkenyl, aryl, heteroaryl, aralkyl, heteroaralkyl, —(CR¹⁵═CR¹⁵)-aryl, or —(CR¹⁵═CR¹⁵)-heteroaryl;
and n is 1, 2, 3, 4, 5 or 6;
or a compound represented by formula XX: 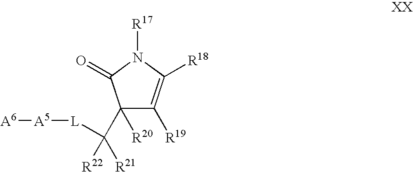
wherein,
R¹⁷ is cycloalkyl, heterocycloalkyl, cycloalkenyl, heterocycloalkenyl, aryl, heteroaryl, aralkyl, heteroaralkyl, or —(C(R²³)₂)ₙ—(CR²³═C(R²³)₂);
R¹⁸ is aryl;
R¹⁹ is hydrogen, alkyl, —CO₂R²⁴, or —C(O)N(R²³)(R²⁴);
R²⁰ and R²¹ represent independently H or alkyl; or R²⁰ and R²¹ taken together form a bond;
R²² and R²³ represent independently for each occurrence H or alkyl;
R²⁴ represents independently for each occurrence alkyl, cycloalkyl, aryl, heteroaryl, aralkyl, or heteroaralkyl;
L is a bond, —C(R²³)₂—, or —(CR²³═CR²³);
A⁵ represents a bivalent cycloalkenyl, heterocycloalkenyl, aryl, heteroaryl, aralkyl, heteroaralkyl, —(CR²³═CR²³)-aryl-, or —(CR²³═CR²³)-heteroaryl-;
A⁶ represents cycloalkenyl, heterocycloalkenyl, heteroaryl, aralkyl, heteroaralkyl, —(CR²³═CR²³)— aryl, or —(CR²³═CR²³)-heteroaryl;
and n is 1, 2, 3, 4, 5 or 6;
or a compound of formula XXI: 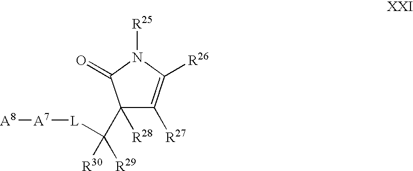
wherein,
R²⁵ is —(C(R³¹)₂)ₙ—(CR³¹═C(R³¹)₂);
R²⁶ is cycloalkyl, heterocycloalkyl, cycloalkenyl, heterocycloalkenyl, heteroaryl, aralkyl, or heteroaralkyl;
R²⁷ is hydrogen, alkyl, —CO₂R³², or —C(O)N(R³¹)(R³²);
R²⁸ and R²⁹ represent independently H or alkyl; or R²⁸ and R²⁹ taken together form a bond;
R³⁰ and R³¹ represent independently for each occurrence H or alkyl;
R³² represents independently for each occurrence alkyl, cycloalkyl, aryl, heteroaryl, aralkyl, or heteroaralkyl;
L is a bond, —C(R³¹)₂—, or —(CR³¹═CR³¹)—;
A⁷ and A⁸ represent independently cycloalkenyl, heterocycloalkenyl, aryl, heteroaryl, aralkyl, heteroaralkyl, —(CR³¹═C R³¹)-aryl, or —(CR³¹═CR³¹)-heteroaryl;
and n is 1, 2, 3, 4, 5 or 6.

77. The compound of claim 76 having formula XVIII, wherein R¹ is aryl.

78. The compound of claim 76 having formula XVIII, wherein R¹ is aryl, and R⁴ and R⁵ taken together form a bond.

79. The compound of claim 76 having formula XVIII, wherein R¹ is aryl, R⁴ and R⁵ taken together form a bond, L is a bond, and A¹ is heteroaryl.

80. The compound of claim 76 having formula XVIII, wherein R¹ is aryl, R⁴ and R⁵ taken together form a bond, L is a bond, A¹ is heteroaryl, and A2 is aryl.

81. The compound of claim 76 having formula XXI, wherein R²⁵ is allyl.

82. The compound of claim 76 having formula XXI, wherein R²⁵ is allyl and R²⁷ is —CO₂R³².

83. The compound of claim 76 having formula XXI, wherein R²⁵ is allyl, R²⁷ is —CO₂R³², and A⁷ is heteroaryl.

84. The compound of claim 76 having formula XXI, wherein R²⁵ is allyl, R²⁷ is —CO₂R³², A⁷ is heteroaryl, and A⁸ is aryl.

85-118. (canceled)

119. A pharmaceutical composition comprising a pharmaceutically acceptable excipient and a compound of claim 76.

120. A method of treating or preventing drug-induced phospholipidosis, comprising the step of:
administering to a patient in need thereof a therapeutically effective amount of a compound of formula III, wherein formula III is represented by: 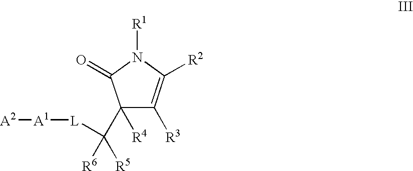
wherein,
R¹ is cycloalkyl, heterocycloalkyl, cycloalkenyl, heterocycloalkenyl, aryl, heteroaryl, aralkyl, heteroaralkyl, or —(C(R⁷)₂)ₙ—(CR⁷═C(R⁷)₂);
R² is cycloalkyl, heterocycloalkyl, cycloalkenyl, heterocycloalkenyl, aryl, heteroaryl, aralkyl, heteroaralkyl, or alkyl;
R³ is hydrogen, alkyl, —CO₂R⁸, or —C(O)N(R⁷)(R⁸);
R⁴ and R⁵ represent independently H or alkyl; or R⁴ and R⁵ taken together form a bond;
R⁶ and R⁷ represent independently for each occurrence H or alkyl;
R⁸ represents independently for each occurrence alkyl, cycloalkyl, aryl, heteroaryl, aralkyl, or heteroaralkyl;
L is a bond, —C(R⁷)₂—, or —(CR⁷═CR⁷)—;
A¹ and A represent independently cycloalkenyl, heterocycloalkenyl, aryl, heteroaryl, aralkyl, heteroaralkyl, —(CR⁷═CR⁷)-aryl, or —(CR⁷═CR⁷)-heteroaryl;
and n is 1, 2, 3, 4, 5 or 6.

121-127. (canceled)

128. The method of claim 120, wherein R¹, R², A¹, and A² represent independently aryl or heteroaryl;
R³ is hydrogen or alkyl; R⁶ is H or alkyl; and L is a bond.

129. The method of claim 120, wherein R¹ is —(C(R⁷)₂)ₙ—(CR⁷═C(R⁷)₂); R² is alkyl; R³ is alkyl, —CO₂R⁸, or —C(O)N(R⁷)(R⁸); R⁴ and R⁵ represent independently H or alkyl; or R⁴ and R⁵ taken together form a bond; R⁶ and R⁷ represent independently for each occurrence H or alkyl; R⁸ represents independently for each occurrence alkyl, cycloalkyl, aryl, heteroaryl, aralkyl, or heteroaralkyl; L is a bond, —C(R⁷)₂—, or —(CR⁷═CR⁷)—; and A¹ and A² represent independently aryl or heteroaryl.

130-143. (canceled)

144. The method of claim 120, wherein said compound is

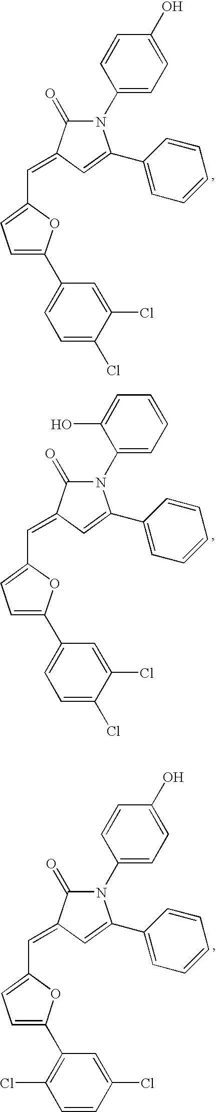

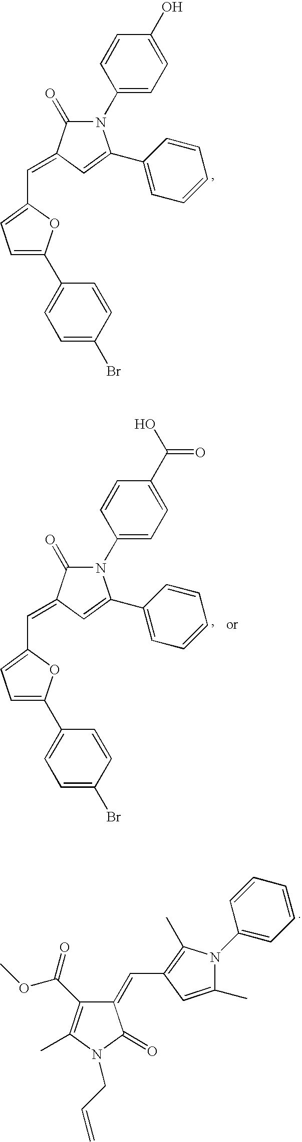

145. The method of claim 120, wherein said patient is a mammal.

146. The method of claim 120, wherein said patient is a human.

147. The method of claim 1, wherein R¹ comprises a carboxylic acid group; R¹ is a carboxylic acid substituted aryl; R¹ is a carboxylic acid substituted phenyl; and/or R¹ is a para-substituted carboxylic acid phenyl.

148. The compound of claim 76 having formula XVIII, wherein R¹ comprises a carboxylic acid group; R¹ is a carboxylic acid substituted aryl; R¹ is a carboxylic acid substituted phenyl; and/or R¹ is a para-substituted carboxylic acid phenyl.

149. The compound of claim 76 having formula XX, wherein R¹⁷ comprises a carboxylic acid group; R¹⁷ is a carboxylic acid substituted aryl; R¹⁷ is a carboxylic acid substituted phenyl; and/or R¹⁷ is a para-substituted carboxylic acid phenyl.

150. The method of claim 29, wherein, R¹ comprises a carboxylic acid group; R¹ is a carboxylic acid substituted aryl; R¹ is a carboxylic acid substituted phenyl; and/or R¹ is a para-substituted carboxylic acid phenyl.

151. The method of claim 120, wherein, R¹ comprises a carboxylic acid group; R¹ is a carboxylic acid substituted aryl; R¹ is a carboxylic acid substituted phenyl; and/or R¹ is a para-substituted carboxylic acid phenyl.
